(19) United States
(12) Patent Application Publication
(10) Pub. No.: US 2025/0313579 A1
(43) Pub. Date: Oct. 9, 2025

(54) LIGAND COMPOUNDS COMPRISING A CHELATING GROUP AS A BRIDGING GROUP

(71) Applicant: Technische Universität München, München (DE)

(72) Inventors: Mara Parzinger, Ravensburg (DE); Lennard Wendlinger, Oberhaching (DE); Hans-Jürgen Wester, Schweitenkirchen (DE)

(21) Appl. No.: 18/293,576

(22) PCT Filed: Aug. 4, 2022
(86) PCT No.: PCT/EP22/71964

§ 371 Date: Jan. 30, 2024

(30) Foreign Application Priority Data
Aug. 5, 2021 (EP) 21189855.6

(51) Int. Cl. C07F 7/12; A61K 51/04; A61K 51/08

(52) U.S. Cl. CPC C07F 7/12; A61K 51/0482; A61K 51/083

(57) ABSTRACT

A compound of formula (I) and its use in therapeutic and diagnostic methods such as radionuclide therapy or nuclear diagnostic imaging.

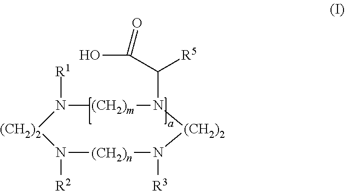

Neuroendocrine tumors (NETs) are a heterogenous group of malignancies, originating from the neuroendocrine system. This system is comprised of neuroendocrine cells in a variety of different tissues like endocrine glands (pituary, parathyroids, adrenal), pancreatic tissue or the endocrine cells located in the digestive and respiratory system (diffuse endocrine system: lungs, gastrointestinal tract).[1] NETs are a rare entity with an incidence of 2-5/100000 (0.5% of newly diagnosed malignancies per year), depending on the patients (ethnic) decent. With 67%, tumors of the gastrointestinal tract are the most common, followed by NETs in the respiratory system with 25%. Even though the incidence may be low, the number of diagnosed entities has increased over the past 30 years due to optimized methods in diagnostics.[1-4]

For diagnostic and therapeutic purposes of NETs, the somatostatin receptor (SST), more precisely, its five subtypes SST₁₋₅ are addressed.[5,6] Those G-protein-coupled receptors are expressed naturally on neuroendocrine cells in different tissues but are overexpressed on various types of NETs and their metastases.[5, 7, 8] Therefore, the SST receptors are attractive targets for diagnostic clarification, applying positron-emission-tomography (PET).[6] Nevertheless, application is not trivial since the expression level of each subtype varies, depending on tumor origin and type. Additionally, numerous ligands may be highly affine for one or two subtypes but are not capable of targeting all SST receptors with sufficient affinity. However, SST₂ is particularly overexpressed on various NETs, therefore it is of high interest for the development of new radiopharmaceuticals.[5, 6]

Among ¹⁸F-based SST tracers, especially [¹⁸F] SiFAlinTATE has gained some interest over the last years.[9, 10] Labeling with ¹⁸F is achieved, through the SiFA-based building block SiFAlin-aldehyde, which contains a permanent positive charge. The in vitro and in vivo parameters have been promising, leading to first in human clinical trials.[11, 12]

Multimodal approaches—the possibility to combine more than one labeling technique within a single peptide or small molecule—have been investigated in different ways.

In recent years, the Chair for Pharmaceutical Radiochemistry at the Technical University of Munich has developed the methodology for radiohybrid (rh) labeling of biomolecules, which allows the labeling of a universal precursor molecule with either ¹⁸F fluoride (for PET) or a trivalent radiometal (such as ⁶⁸Ga³⁺ for PET, ¹⁷⁷Lu³⁺ for the PRRT). When a rh ligand is labeled with ¹⁸F fluoride, the cold metal can be complexed in the molecule-when labeled with a radiometal, cold ¹⁹F fluorine is present. Therefore, the ¹⁸F-labeled peptide and the corresponding radiometal-labeled analog possess the same chemical structure and thus identical in vitro and in vivo properties, thereby allowing the generation of structurally identical theranostic tracers with exactly the same in vivo properties of the diagnostic and therapeutic tracers (eg ¹⁸F/¹⁷⁷Lu analogs).

The combination of a chelator and another modality for a different labeling approach can be applied in many ways, therefore different multimodal approaches have been investigated in the past. Schottelius et al. combined the already established PSMA ligand PSMA I&T with the fluorescent dye sulfo-Cy5 resulting in a fluorescence-radiohybrid structure (PSMA I&F).[13] Roxin et al. designed their own version of the radiohybrid concept: a VLA-4 targeting peptide comprised of the chelator DOTA and a BF₃-based structure (DOTA-AMBF₃-LLP2A). Analogously to the already introduced radiohybrid concePt, DOTA-AMBF₃-LLP2A can also be labeled with ¹⁸F and a trivalent radiometal (first investigations were limited to the uncomplexed compound).[14]

Frequently, the two modalities are conjugated via a trivalent unit e.g. diaminoproprionic acid (rhPSMA7) or a lysine unit (PSMA I&F, DOTA-AMBF₃-LLP2A), usually resulting in sterically demanding radiohybrid or fluorescent-radiohybrid moieties.

A different approach was chosen by Gai et al., They designed more complex DOTA- and NOTA-based building blocks, which are directly introducible into the peptide backbone, either via standard peptide chemistry or by applying a combination of peptide and click chemistry.[15]

The chelator DOTPI has been used to generate the symmetrical tetrameric PSMA ligands DOTPI(Trz-KuE)₄ and DOTPI(DBCO-KuE)₄ or as bridging unit in the αvβ3 integrin addressing tetramer DOTPI(RGD) 4.[16, 17] Analogous examples are described for multivalent TRAP peptides. Additionally, a multimodal approach has been published, wherein a dimeric TRAP conjugate, is also equipped with the fluorophore rhodamine 6G for fluorescence applications.

The application of chelators like TRAP and DOTPI as multimeric bridges results usually in peptides of high affinity, due to the general concept of avidity.[19] The combination of carboxylates for the conjugation with target addressing peptides and hydrophilic phosphinates for the complexation of radiometals results in peptides of overall high hydrophilicity.[16, 18]

Although typical parameters as target affinity and lipophilicity are generally promising, the synthetical accessibility of the chelators themselves as well as the multimeric/multimodal peptides is complicated and often unfavorable.

The present invention provides a novel approach for the development of chelator-based radiohybrid ligand compounds. In these compounds, the heterocyclic ring structure of a chelator functions as a bridging structure between the binding motif and a SiFA group as a second labeling structure. Since the chelating structure serves as a linker, an additional linker structure acting as a spacer between the binding motif and the chelator is not needed, so that the overall structure of the ligand compound is simplified. The resulting compounds are of high affinity, high hydrophilicity and low binding to human serum albumin, resulting in favorable in vivo results in the mouse model.

In particular, the invention provides a compound selected from:
a compound of the following formula (I):

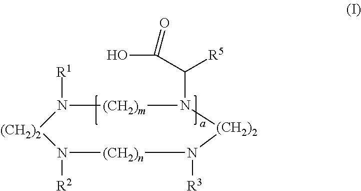

wherein
a is 0 or 1, preferably 1;
m is 2 or 3, preferably 2;
n is 2 or 3, preferably 2;
one group selected from R¹, R² and R³ is a group comprising an effector moiety R^{B};
another group selected from R¹, R² and R³ is a group comprising a silicon-based fluoride acceptor (SiFA) moiety R^{S} which moiety comprises a silicon atom and a fluorine atom, wherein the fluorine atom is linked via a covalent bond directly to the silicon atom, and which can be labeled with ¹⁸F by isotopic exchange of ¹⁹F by ¹⁸F or which is labeled with ¹⁸F;
and the remaining group selected from R¹, R² and R³ is a group of the formula (R-1):

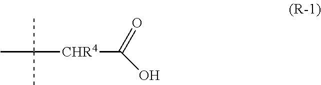

wherein
R⁴ is selected from —H, —OH and C1-C3 alkyl, and is preferably —H; and wherein the dashed line marks a bond which attaches the group to the remainder of the compound;
R⁵ is selected from —H, —OH and C1-C3 alkyl, and is preferably —H;
a salt thereof,
and a chelate compound formed from a compound of formula (I) or its salt and a radioactive or non-radioactive cation.

As explained above, the compounds of the invention are selected from compounds of formula (I), their salts (i.e. salts of the compound of formula (I), typically pharmaceutically acceptable salts), and chelate compounds formed from a compound of formula (I) or its salt and a radioactive or non-radioactive cation. Thus, unless indicated to the contrary, any reference to a compound of the invention herein encompasses the compounds of formula (I) (and the preferred embodiments of this formula disclosed herein), the salts thereof, and the chelate compounds. Likewise, any racemates, enantiomers, or diastereomers of any chiral compounds of formula (I) and their salts are encompassed, unless a specific stereochemistry of the compound under consideration is indicated in a specific context. The compounds of the invention may also be referred to herein as ligand compounds of the invention, or briefly as ligands.

In the following, the structural elements of the compounds of the invention shall be further discussed. As will be understood by the skilled reader, information which is provided in this context about the (preferred) structure of the compounds of formula (I) also applies for the salts of the compounds of formula (I) and the chelate compound formed from a compound of formula (I) or its salt and a radioactive or non-radioactive cation.

In formula (I), a is 0 or 1, and is preferably 1. Thus, it is preferred that the compounds of formula (I) are compounds of formula (IA):

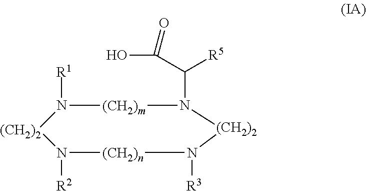

wherein the variables m, n and R¹ to R⁵ are defined as above.

As illustrated by formula (I), the compounds of the invention comprise a substituted heterocycle which includes 3 nitrogen atoms (if a is 0) or 4 nitrogen atoms (if a is 1) as ring members. The nitrogen atoms present as ring members in the heterocycle are linked via ethanediyl groups —CH₂—CH₂-(if m is 2 and n is 2), or by ethanediyl groups and one or two propanediyl groups —CH₂—CH₂—CH₂-(if m is 3, n is 3 or both of m and n are 3). The heterocycle formed by the nitrogen atoms and the ethanediyl groups or the ethanediyl groups and (a) propanediyl group(s) is also referred to herein as nitrogen containing macrocycle.

As will be understood by the skilled reader, if a is 0, the moiety contained in the brackets [ . . . ] carrying the index a in formula (I) is absent, and a direct bond is formed between the nitrogen atom carrying the substituent-R¹ and the —CH₂—CH₂—group shown on the two sides of the moiety in brackets in the formula.

In view of the preferences for a, m and n indicated above, it will be understood that the combination a=1, m=2 and n=2 is a further preferred combination for the compounds of formula (I), as illustrated in the following preferred formula (IB):

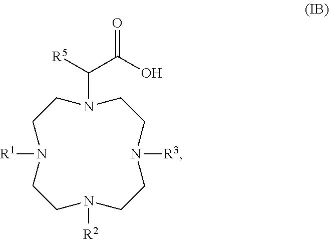

wherein the variables R¹ to R³ and R⁵ are defined as above.

In formula (I) and its preferred embodiments, one group selected from R¹, R² and R³ (i.e. either R¹ or R² or R³) comprises an effector moiety R^{B}. A preferred example of such an effector moiety R^{B} is a binding motif which allows a ligand/receptor interaction to take place between the compounds in accordance with the invention and a receptor of therapeutic and/or diagnostic interest. A preferred example of such a receptor is a somatostatin (SST) receptor. Such a binding motif can serve as a fundamental affinity anchor for the compounds towards the receptor. More preferably, R^{B} is a binding motif which is able to bind to at least somatostatin receptor 2, or SST₂, or to more somatostatin receptor subtypes, or even to all somatostatin receptor subtypes, the latter resulting in so called SST pan-receptor ligands.

If R^{B} represents a binding motif in line with the above, it is generally capable of binding with high affinity to a receptor. In this context, high affinity binding preferably means that the compound comprising the binding motif exhibit an IC50 in the low nanomolar range, preferably 50 nM or less, more preferably 10 nM or less, still more preferably 5 nM or less. For the sake of clarity, the half maximal inhibitory concentration (IC50) is defined here as the quantitative measure of the molar concentration of binding motif R^{B} or a compound according to the invention comprising it which is necessary to inhibit the binding of a radioactive reference ligand to a receptor in vitro by 50%. For example, as a reference ligand for the binding to SST receptors, [¹²⁵I] Tyr³-Octreotide may be relied on.

It will be understood that a preferred binding motif as an effector moiety which is capable of high affinity binding to an SST receptor as referred to herein may show high affinity to more than one SST receptor type. Preferably, the binding moiety R^{B} is one which shows the highest binding affinity among SST receptor subtypes to SST₂.

Suitable binding motifs include agonists and antagonists of an SST receptor.

The effector molecule R^{B} generally comprises a coupling group, i.e. a functional group which allows R^{B} to be attached to the remainder of the compound of the invention via a covalent bond. The coupling group may consist of one or more atoms. Exemplary coupling groups can be selected from —NH—, —NR—, wherein the group R is C1 to C6 alkyl, and is preferably methyl, —C(O)—, —O—, —S—, a quaternary ammonium group, and a thiourea bridge or a group which forms such a thiourea bridge together with a complementary group to which R^{B} is attached. In this context, and also in other instances where reference is made to a quaternary ammonium group as a possible coupling group herein, the quaternary ammonium group is preferably a coupling group of the formula —N(R)₂+—, wherein the groups R are independently C1 to C6 alkyl, and are preferably methyl. As will be understood, a coupling group comprised by R^{B} may be covalently linked to a further, complementary coupling group comprised by the compound in accordance with the invention, so that the two coupling groups combine to form a binding unit, such as an amide bond (—C(O)—NH—), an alkylated amide bond (—C(O)—NR—), or a thiourea bidge (—NH—C(S)—NH—). As referred to herein, also in further instances below, the substituent R in the alkylated amide bond —C(O)—NR—is C1 to C6 alkyl, preferably methyl. It is preferred that R^{B} comprises a coupling group —NH—, and that the coupling group forms an amide bond —C(O)—NH—with a group —C(O)—contained in the compound in accordance with the invention. For example, in formulae (R-2a), (R-2b), (IC) and (ID) disclosed herein, it is preferred that R^{B} comprises a coupling group —NH—or—NR—, preferably —NH—, and that the coupling group is bound to the —C(O)—group to which R^{B} is attached in these formulae to form an amide bond (—C(O)—NH—) or an alkylated amide bond (—C(O)—NR—), preferably an amide bond.

Preferably, the effector moiety R^{B} is a peptidic binding motif, i.e. a binding motif which comprises a peptide structure which is able to bind to a receptor. The peptidic binding motif preferably comprises a cyclic peptide structure or a peptide cyclized by a disulfide bridge. As noted above, the binding motif is preferably one which is capable of binding to an SST. Diverse peptides capable of binding to an SST are known and described in the literature. They can be used to provide the group R^{B} in a compound of the invention, e.g. by forming an amide bond with the remainder of the compound using a carboxylic acid group or an amino group contained in the peptide.

Thus, R^{B} may comprise a group, and preferably is a group, which can be derived from a receptor agonist or receptor antagonist selected from Tyr³-Octreotate (TATE, H-D-Phe-cyclo(L-Cys-L-Tyr-D-Trp-L-Lys-L-Thr-L-Cys)-L-Thr-OH), Thr⁸-Octreotide (ATE), Phe1-Tyr³-Octreotide (TOC, H-D-Phe-cyclo(L-Cys-L-Tyr-D-Trp-L-Lys-L-Thr-L-Cys)-L-Thr-ol), NaI³-Octreotide (NOC, H-D-Phe-cyclo(L-Cys-L-1-Nal-D-Trp-L-Lys-L-Thr-L-Cys)-L-Thr-ol), 1-NaI³, Thr⁸-Octreotide (NOCATE), BzThi3-Octreotide (BOC), BzThi3, Thr⁸-Octreotide (BOCATE), JR11 (H-L-Cpa-cyclo(D-Cys-L-Aph(Hor)-D-Aph (Cbm)-L-Lys-L-Thr-L-Cys)-D-Tyr-NH2), BASS (H-L-Phe (4-NO₂)-cyclo(D-Cys-L-Tyr-D-Trp-L-Lys-L-Thr-L-Cys)-D-Tyr-NH2) and KE121 (cyclo(D-Dab-L-Arg-L-Phe-L-Phe-D-Trp-L-Lys-L-Thr-L-Phe)), more preferably from TATE or JR11, and most preferably from TATE. As will be understood by the skilled reader, the group R^{B} can be conveniently derived from the receptor agonists or antagonists listed above by using a functional group, such as a carboxylic acid group or an amino group, contained in the receptor agonist or antagonist, to provide a coupling group which attaches the group R^{B} to the remainder of the compound. Preferably, these peptidic receptor agonists or receptor antagonists provide the group R^{B} by using an amino group contained therein, e.g. in an optionally substituted phenylalanine unit contained in the peptide, to form an amide bond with the remainder of the compound of the invention. For example, in formulae (R-2a), (R-2b), (ID), (IE), (IF) and (IG) disclosed herein, the covalent bond between R^{B} and the carbonyl group —C(O)-to which R^{B} is attached may be formed using an—NH-coupling group derived from an amino group contained in the above receptor agonists or receptor antagonists.

Alternatively, as will be understood by the skilled reader, the group R^{B} can be conveniently derived from the receptor agonist or receptor antagonist listed above by the introduction of an additional functional moiety into the group R^{B} which provides a functional group that allows a chemical bond to be formed to the remainder of the compound of the invention, such as a moiety with an isothiocyanate that can link to an amine to form a thiourea bridge. As will be understood by the skilled reader, other conjugation strategies, typically summarized as “bioconjugation strategies” can also be used to link a group R^{B} in a compound in accordance with the invention to the remainder of the compound in accordance with the invention.

In line with the above, it is preferred that R^{B} is a group of the formula (B-1):

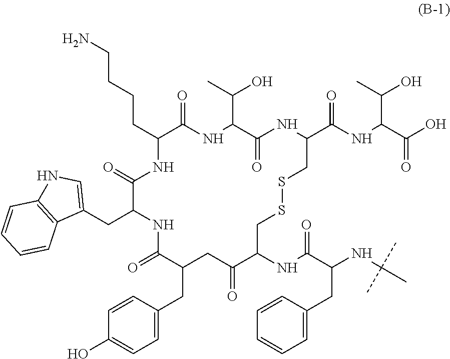

wherein the dashed line marks a bond which attaches the group to the remainder of the compound. As will be understood by the skilled reader, the bond marked by the dashed line in formula (B-1) does not carry a methyl group at its end opposite to the nitrogen atom, but represents a bond which attaches the group R^{B} to the remainder of the compound of formula (I). Preferably, the bond marked by the dashed line in formula (B-1) represents a covalent bond which is present in a compound of the invention between the nitrogen atom of the —NH-group indicated in formula (B-1) and a carbon atom of a carbonyl group to which R^{B} may be attached, e.g. as in formulae (R-2a), (R-2b), (ID), (IE), (IF) and (IG) disclosed herein. Thus, an amide bond can be provided.

More preferably, R^{B} is a group of the formula (B-1a):

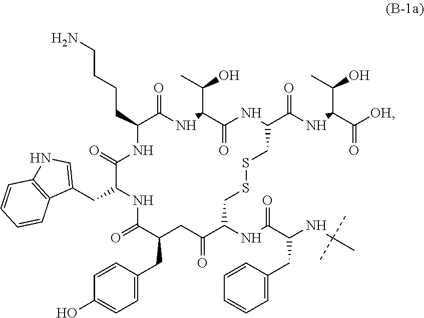

wherein the dashed line marks a bond which attaches the group to the remainder of the compound.

Preferably, the group selected from R¹, R² and R³ which is the group comprising an effector moiety R^{B} is a group of the formula (R-2a) or (R-2b), more preferably of the formula (R-2a):

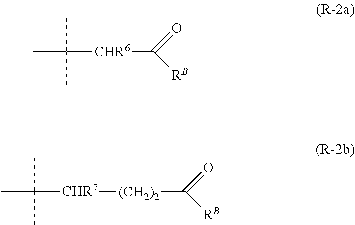

wherein
R^{B} is the effector moiety as defined herein, including any preferred embodiments thereof;
R⁶ is selected from —H, —OH and C1-C3 alkyl, and is preferably —H; and
R⁷ is —COOH;
and wherein the dashed line marks a bond which attaches the group to the remainder of the compound. Thus, as will be understood by the skilled person, the bond marked with the dashed line does not carry a methyl group at its end opposite to the group CHR⁶ and CHR⁷, respectively, but represents a covalent bond which is present in a compound of the invention between the group CHR⁶ or CHR⁷, respectively, and the nitrogen atom in formula (I) or its preferred embodiments to which the group selected from R¹, R² and R³ which is the group comprising an effector moiety R^{B} is attached.

Another group selected from R¹, R² and R³, i.e. one of the two groups which are not the group comprising the moiety R^{B} discussed above, is a group comprising a silicon-based fluoride acceptor (SiFA) moiety R^{S}. Such a SiFA moiety comprises a silicon atom and a fluorine atom, and the fluorine atom is linked via a covalent bond directly to the silicon atom. The SiFA moiety can be labeled with ¹⁸F by isotopic exchange of ¹⁹F by ¹⁸F, or is labeled with ¹⁸F.

Preferably, the SIFA moiety R^{S} comprises a group of formula (S-1):

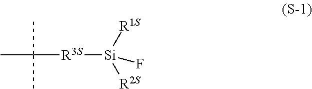

wherein
R^{1S} and R^{2S} are independently from each other a linear or branched C3 to C10 alkyl group, preferably R^{1S} and R^{2S} are selected from isopropyl and tert-butyl, and more preferably RIS and R^{2S} are tert-butyl; and
R^{3S} is a divalent C1 to C20 hydrocarbon group which comprises one or more aromatic and/or aliphatic moieties, and which optionally comprises up to 3 heteroatoms selected from O and S, preferably R^{3S} is a divalent C6 to C12 hydrocarbon group which comprises an aromatic ring and which may comprise one or more aliphatic moieties;
and wherein the dashed line marks a bond which attaches the group to the remainder of the compound.

More preferably, the SiFA moiety R^{S} comprises a group of the formula (S-2):

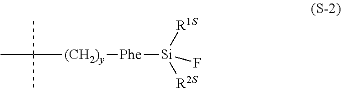

wherein
R^{1S} and R^{2S} are independently from each other a linear or branched C3 to C10 alkyl group, preferably R^{1S} and R^{2S} are selected from isopropyl and tert-butyl, and more preferably R^{1S} and R^{2S} are tert-butyl, Phe is a phenylene group, y is an integer of 0 to 6, preferably 0 or 1 and more preferably 1, and wherein the dashed line marks a bond which attaches the group to the remainder of the compound. The two substituents on the phenylene group are preferably in para-position to each other. It is particularly preferred that the group R^{S} comprises a group of formula (S-2) wherein R^{1S} and R^{2S} are tert-butyl, and wherein y is 1.

Together with the Si and the F atom, preferably in the form of a group as shown above, the SiFA group R^{S} may comprise a coupling group which allows R^{S} to be attached to the remainder of the compound of the invention via a covalent bond which is formed between the group R^{S} and its point of attachment in formula (I). The coupling group may consist of one or more atoms. Exemplary coupling groups are selected from —NH—, —NR—, —C(O)—, —O—, —S—, —N(R)₂+-(CH₂)—C(O)—, and a thiourea bridge or a group which forms such a thiourea bridge together with a complementary group to which R^{S} is attached. In the above exemplary groups, R is C1 to C6 alkyl, and is preferably methyl, and r is 1, 2, or 3, and is preferably 1. The coupling group may be covalently linked to a further, complementary coupling group provided in the compound of the invention at the point of attachment of R^{S}, so that the two coupling groups combine to form a binding unit, such as an amide bond —C(O)—NH—, an alkylated amide bond —C(O)—NR—, or a thiourea bridge-NH—C(S)—NH—, preferably an amide bond. Preferred as a coupling group are —C(O)—and —N(R)₂+-(CH₂)—C(O)—. Likewise, it is preferred that these coupling groups comprised by R^{S} form an amide bond with a complementary coupling group provided in the compound of the invention at the point of attachment of R^{S}.

Alternatively, the group R^{S} may be attached to the remainder of the compound of the invention by a covalent bond formed to a quaternary ammonium group as a coupling group that is provided at the point of attachment of R^{S} in the compound of formula (I). As noted above, the quaternary ammonium group is preferably a coupling group of the formula —N(R)₂⁺—, wherein the groups R are independently C1 to C6 alkyl and are preferably methyl. As will be understood by the skilled reader, this may be accomplished e.g. if the unit carrying R^{S} is provided using a compound with a tertiary amino group, which is converted to a quaternary amino group upon conjugation with the SiFA group.

In line with the above, it is particularly preferred that the SiFA moiety R^{S} is a group of the formula (S-3):

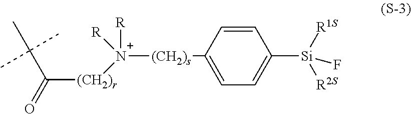

wherein
r is 1, 2 or 3, preferably 1, s in-(CH₂)ₛ- is an integer of 1 to 6 and is preferably 1,
R is, independently, C1 to C6 alkyl and is preferably methyl, and
R^{1S} and R^{2S} are independently from each other a linear or branched C3 to C10 alkyl group, preferably R^{1S} and R^{2S} are selected from isopropyl and tert-butyl, and more preferably RIS and R^{2S} are tert-butyl; and wherein the dashed line marks a bond which attaches the group to the remainder of the compound.

Further in line with the above, the group of formula (S-3) and thus the SiFA moiety R^{S} is most preferably a group of the formula (S-4):

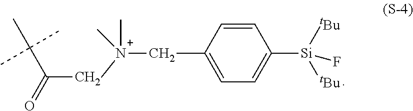

wherein ^{t}Bu indicates a tert-butyl group and the dashed line marks a bond which attaches the group to the remainder of the compound.

As will be understood by the skilled person, the bond marked by the dashed line in formula (S-3) and (S-4) does not carry a methyl group at its end opposite to the —C(O)—group, but rather serves to attach the group to the remainder of the compound. Preferably, the bond marked by the dashed line in formulae (S-3) and (S-4) represents a covalent bond which is present in a compound of the invention between the carbon atom of the —C(O)—group indicated in formulae (S-3) and (S-4) and a nitrogen atom of a —NH—group which may be provided at the point of attachment of R^{S} in the compounds of the invention, e.g. a —NH—group which may be contained in the linking group L^{D} present in formula (R-3a), (R-3c), or (ID) shown below to which R^{S} is attached in the respective formulae, or a —NH—group which may be contained in the linking group L^{T} present in formula (R-3b), (R-3d), or (IE) shown below to which R^{S} is attached in the respective formulae, or the —NH—group contained in formula (IF) or (IG) to which R^{S1} is attached. Thus, an amide bond can be provided as a binding unit.

Exemplary counterions for the positively charged quaternary ammonium group indicated in formula (S-3) and (S-4) which carries two substituents R (in formula (S-3)) or two methyl substituents (in formula (S-4)), respectively, are anions as they are discussed herein with regard to salts forms of the compound of formula (I), which include, e.g., trifluoro acetate anions or acetate anions.

The fluorine atom indicated in formulae (S-1) to (S-4) may be a ¹⁸F atom, or a ¹⁹F atom which can be exchanged to provide ¹⁸F by isotopic exchange of ¹⁹F by ¹⁸F.

Preferably, the group selected from R¹, R² and R³ which is the group comprising the SiFA moiety R^{S} is a group of the formula (R-3a), (R-3b), (R-3c) or (R-3d), more preferably of the formula (R-3a) of (R-3b).

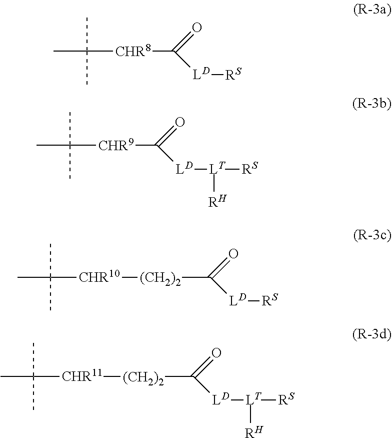

wherein
R^{S} is the SiFA moiety as defined herein, including any preferred embodiments thereof;
R⁸ and R⁹ are selected from —H, —OH and C1-C3 alkyl, and are preferably —H;
R¹⁰ and R¹¹ are —COOH;
L^{D} is a divalent linking group;
L^{T} is a trivalent linking group;
R^{H} is a hydrophilic modifying group;
and the dashed line marks a bond which attaches the group to the remainder of the compound. Thus, as will be understood by the skilled person, the bond marked with the dashed line does not carry a methyl group at its end opposite to the group CHR⁸, CHR⁹, CHR¹⁰, and CHR¹¹, respectively, but represents a covalent bond which is present in a compound of the invention between the group CHR⁸, CHR⁹, CHR¹⁰, or CHR¹¹, respectively, and the nitrogen atom in formula (I) or its preferred embodiments to which the group selected from R¹, R² and R³ which is the group comprising a SiFA moiety R^{S} is attached.

The remaining group selected from R¹, R² and R³ (i.e. the group which is neither the group comprising the effector moiety R^{B}, nor the group comprising the SiFA moiety) is a group of the formula (R-1):

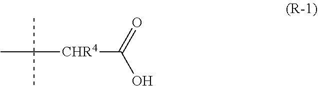

wherein
R⁴ is selected from —H, —OH and C1-C3 alkyl, and is preferably —H; and wherein the dashed line marks a bond which attaches the group to the remainder of the compound. Thus, as will be understood by the skilled person, the bond marked by the dashed line in formula (R-1) does not carry a methyl group at its end opposite to the group CHR⁴, but rather serves to attach the group to a nitrogen atom shown in formula (I) or is preferred embodiments.

In line with the above, various exemplary combinations of R¹, R² and R³ are encompassed by formula (I) and (IA), as listed in the following table.

| No. | R¹ is . . . | R² is . . . | R³ is . . . |
|---|---|---|---|
| 1 | group comprising R^{B} | group comprising R^{S} | group (R-1) |
| 2 | group comprising R^{B} | group (R-1) | group comprising R^{S} |
| 3 | group comprising R^{S} | group comprising R^{B} | group (R-1) |
| 4 | group (R-1) | group comprising R^{B} | group comprising R^{S} |
| 5 | group comprising R^{S} | group (R-1) | group comprising R^{B} |
| 6 | group (R-1) | group comprising R^{S} | group comprising R^{B} |

It will be understood that the reference to the “group comprising R^{B}” and the “group comprising R^{S}” in the table encompasses the preferred variants of these groups and of R^{B} and R^{S} themselves.

Preferred among these exemplary combinations are combination No. 2 and No. 5. Thus, particularly preferred are combinations No. 2 and No. 5, wherein a is 1.

In line with the above, the compound of formula (I) is preferably a compound of formula (IC):

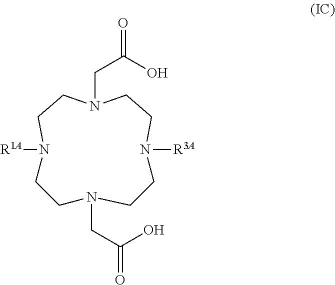

wherein
i) R^{1A} is a group of formula (R-2a) as defined herein and R^{3A} is selected from the groups of formula (R-3a), (R-3b), (R-3c) and (R-3d) as defined herein; or
ii) R^{1A} is selected from the groups of formula (R-2a) and (R-2b) as defined herein and R^{3A} is selected from the groups of formula (R-3a) and (R-3b) as defined herein.

Moreover, the compound of formula (I) is more preferably a compound of formula (ID) or (IE):

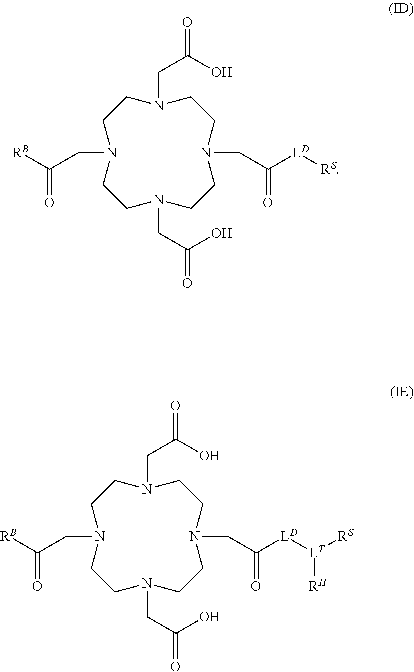

wherein
R^{B} is the effector moiety as defined herein, including any preferred embodiments thereof, R^{S} is the SiFA moiety as defined herein, including any preferred embodiments thereof,
L^{D} is a divalent linking group;
L^{T} is a trivalent linking group; and
R^{H} is a hydrophilic modifying group.

Thus, it will be understood that also in those cases where the compound of formula (I) is a compound of formula (IC), or preferably a compound of formula (ID) or (IE), it is further preferred in formula (IC), (ID) and (IE) that
R^{B} is a moiety which can be derived from a receptor agonist or receptor antagonist selected from Tyr³-Octreotate (TATE, H-D-Phe-cyclo(L-Cys-L-Tyr-D-Trp-L-Lys-L-Thr-L-Cys)-L-Thr-OH), Thr⁸-Octreotide (ATE), Phe1-Tyr³-Octreotide (TOC, H-D-Phe-cyclo(L-Cys-L-Tyr-D-Trp-L-Lys-L-Thr-L-Cys)-L-Thr-ol), NaI³-Octreotide (NOC, H-D-Phe-cyclo(L-Cys-L-1-Nal-D-Trp-L-Lys-L-Thr-L-Cys)-L-Thr-ol), 1-NaI³, Thr⁸-Octreotide (NOCATE), BzThi3-Octreotide (BOC), BzThi3, Thr⁸-Octreotide (BOCATE), JR11 (H-L-Cpa-cyclo(D-Cys-L-Aph(Hor)-D-Aph (Cbm)-L-Lys-L-Thr-L-Cys)-D-Tyr-NH2), BASS (H-L-Phe (4-NO₂)-cyclo(D-Cys-L-Tyr-D-Trp-L-Lys-L-Thr-L-Cys)-D-Tyr-NH2) and KE121 (cyclo(D-Dab-L-Arg-L-Phe-L-Phe-D-Trp-L-Lys-L-Thr-L-Phe); and R^{S} is a group of the formula (S-3) as defined above, but wherein R^{1S} and R^{2S} are both tert-butyl

Still more preferably in formula (IC), (ID) and (IE), R^{B} is a group of formula (B-1a) as defined above, and R^{S} is a group of formula (S-4) as defined above.

The group L^{D} shown in the above formulae (R-3a), (R-3b), (R-3c), (R-3d), (ID) and (IE) is a divalent linking group. The divalent linking group L^{D} may comprise, e.g., a —NH—group or a group —NR-(wherein R is C1-C6 alkyl, preferably methyl), as a coupling group at each of its two termini for attachment to adjacent groups. More preferably, each of the groups —NH—or—NR-combines with a carbonyl group (—C(O)—) as an adjacent group to form an amide bond —NH—C(O)—or an alkylated amide bond-NR—C(O)—. Among the groups —NH—and —NR—, preference is given to—NH—. For example, the linking group L^{D} may comprise or consist of a group —NH—R^{L1}—NH—, wherein R^{L1} is an alkanediyl group, such as a C1-C6 alkanediyl group, and wherein the alkanediyl group may carry one or more, such as one, two or three, substituents selected from —OH, —COOH, —CONH₂, or —NH₂.

Preferably, the divalent linking group L^{D} comprises or consists of a group (L-1):

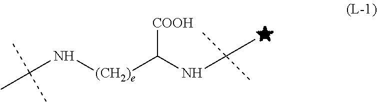

wherein e is an integer of 1 to 6, preferably 1 to 4, the dashed lines mark bonds which attach the group to adjacent groups, and the bond additionally marked by the asterisk is preferably attached to R^{S} or L^{T}, respectively. Such a group (L-1) can be conveniently derived from an amino acid selected from diaminopropionic acid (Dap), diaminobutyric acid (Dab), ornithine (Orn) and lysine (Lys) by using the —NH₂ groups contained in these amino acids to provide a coupling group —NH—wherein the bond to one hydrogen atom in the —NH₂ group is replaced by a bond to another adjacent atom or group. If the group (L-1) is present and is derived from an amino acid as mentioned above, the amino acid is preferably in D-configuration.

The divalent linking group L^{D} may also comprise or consist of one or more hydrophilic units selected from a carbohydrate unit, a polyvalent alcohol unit, a polyvalent carboxylic acid unit and an amino acid unit derived from a hydrophilic amino acid which comprises a further hydrophilic functional group in addition to its —NH₂ and its —COOH functional group. As will be understood by the skilled person, also in this context, the units are named by the chemical structures form which they are derived. For example, these one or more hydrophilic units may be combined with the group of formula (L-1) to provide the linking group L^{D}.

In line with the above, a preferred structure of the divalent linking group L^{D} is a group of formula (L-2):

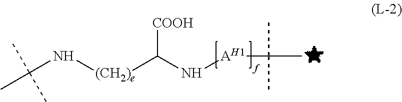

wherein
e is an integer of 1 to 6, preferably 1 to 4,
f is an integer of 0 to 5, preferably 0 or 1,
A^{H1} is, independently for each occurrence if f is more than 1, an amino acid unit derived from a hydrophilic amino acid which comprises a further hydrophilic functional group in addition to its —NH₂ and its —COOH functional group, the dashed lines mark bonds which attach the group to adjacent groups, and the bond additionally marked by the asterisk is attached to R^{S} or RT, respectively.

A^{H1} is an amino acid unit. As will be understood by the skilled person, an amino acid unit is a group which can be derived from an amino acid, i.e. from a compound comprising an amino group and a carboxylic acid group in the same molecule. A specific amino acid unit is typically identified by the name of the amino acid from which it can be derived, e.g. as a ornithine unit, lysine unit, etc. Unless indicated otherwise in a specific context, the amino acids from which the amino acid units can be derived are preferably α-amino acids. If an amino acid unit can be derived from a chiral amino acid, preference is given to the D-configuration.

As will be further understood, an amino acid unit can be derived from an amino acid by using one or more of its functional groups to provide a coupling group which forms a bond to an adjacent atom or group to which the amino acid unit is attached. For example, an amino group of the amino acid may be used to provide a coupling group —NH—wherein the bond to one hydrogen atom in the amino group is replaced by a bond to another adjacent atom or group. A carboxylic acid group of the amino acid may be used to provide a coupling group —C(O)—wherein the bond to the —OH group is replaced by a bond to another adjacent atom or group. Preferably, any coupling group provided by the amino acid is covalently linked to a further, complementary coupling group in the compound in accordance with the invention, so that the two complementary coupling groups combine to form a binding unit, such as an amide bond (—C(O)—NH—) or an alkylated amide bond —C(O)—NR—, preferably an amide bond. R is C1 to C6 alkyl, preferably methyl.

Specifically, in formula (L-2), A^{H1} is, independently for each occurrence if f is more than 1, an amino acid unit derived from a hydrophilic amino acid which comprises, in addition to its —NH₂ and its —COOH functional group, a further hydrophilic functional group. Such a unit may be briefly referred to herein as “hydrophilic amino acid unit”.

For example, the further hydrophilic functional group of the amino acid unit(s) A^{H1} can be selected, independently for each occurrence if f is more than 1, from —NH₂, —COOH, —NH—C(═NH)—NH₂, —C(═O) NH₂, —NH—C(═O)—NH₂, —OH and —P(═O)(OH)₂.

Preferably, each of the f amino acid unit(s) A^{H1} comprises, independently for each occurrence if f is more than 1, a side chain having a terminal hydrophilic functional group which side chain is selected from —(CH₂)ᵥ—NH₂, —(CH₂)ᵥ—COOH, —(CH₂)ᵥ—NH—C(═NH)—NH₂, —(CH₂)ᵥ—C(═O) NH₂, —(CH₂)ᵥ—NH—C(═O)—NH₂, —(CH₂)ᵥ—OH and —(CH₂)ᵥ—P(═O)(OH)₂ wherein v is 1 to 4.

Thus, it is preferred that the amino acid unit(s) A^{H1} is (are) selected, independently for each occurrence if f is more than 1, from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, serine (Ser) unit, citrulline (Cit) unit, thiocitrullin unit, methylisothiocitrulline unit, canavanin unit, thiocanavanin unit, α-amino-γ-(thioureaoxy)-n-butyric acid unit, α-amino-γ-(thioureathia)-n-butyric acid unit, and a phosphonomethylalanine (Pma) unit. They are preferably units which can be derived from amino acids in D-configuration. Particularly preferred are units (is a unit) selected from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, serine (Ser) unit, a citrulline (Cit) unit and a phosphonomethylalanine (Pma) unit. Thus, for example, a preferred group [A^{H1}], wherein f is 1 may be provided by an Asp unit or by a Glu unit.

Preferably, the group —[A^{H1}]_{f}-provides a C-terminus which forms an amide bond with the NH group to which the group —[A^{H1}]_{f}- is attached in formula (L-2), and an N-terminus which forms an amide bond with L^{T} or R^{S}, respectively.

In line with the above, the unit-[A^{H1}]_{f}, - is preferably a unit of the formula:

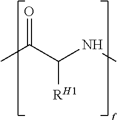

wherein f is as defined above. Each of the f groups R^{H1}, independently for each occurrence if f is more than 1, is selected from
—(CH₂)ᵥ—NH₂, —(CH₂)ᵥ—COOH, —(CH₂)ᵥ—NH—C(═NH)—NH₂, —(CH₂)ᵥ—C(═O) NH₂, —(CH₂)ᵥ—NH—C(═O)—NH₂, —(CH₂)ᵥ—OH and —(CH₂)ᵥ—P(═O)(OH)₂ wherein v is 1 to 4.

It is more preferred that the amino acid unit(s)—C(O)—CH(R^{H1})—NH—of the above formula is (are) selected, independently for each occurrence if f is more than 1, from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gin) unit, serine (Ser) unit, a citrulline (Cit) unit and a phosphonomethylalanine (Pma) unit. They are preferably units which can be derived from amino acids in D-configuration. Thus, for example, a preferred group —[C(O)—CH(R^{H1})—NH]_{f}—wherein f is 1 may be provided by an Asp unit or by a Glu unit.

The C-terminus of the group —[C(O)—CH(R^{H1})—NH]_{f}-generally forms an amide bond with the NH group to which the group —[A^{H1}] f- is attached in formula (L-2), and the N-terminus preferably forms an amide bond with L^{T}, or R^{S}, respectively.

The group L^{T} shown in the above formulae (R-3b), (R-3d) and (IE) is a trivalent linking group.

Preferably, L^{T} is a trivalent amino acid unit, i.e. a unit derived from an amino acid comprising a further functional group in addition to the amino group and the carboxylic acid group required for an amino acid. It is preferred that the further functional group is also an amino or a carboxylic acid group, and that the unit is attached in the compound of the invention with three amide bonds formed using an amino group, a carboxylic acid group and the further functional group provided by the amino acid from which the amino acid unit is derived.

More preferably, L^{T} is a trivalent amino acid unit selected from the following (i) and (ii), with (i) being preferred:
(i) a trivalent amino acid unit which can be derived from an amino acid comprising together with the carboxylic acid group and the amino group a further functional group selected form a carboxylic acid group and an amino group.
(ii) a trivalent amino acid unit comprising a —N(R)₂+- group which unit can be derived from a trifunctional amino acid comprising a tertiary amino group as a third functional group in addition to its —NH₂ group and its —COOH group, and wherein Ris, independently, C1-C6 alkyl, preferably methyl.

For example, the trivalent amino acid unit in line with (i) above which can be derived from an amino acid comprising together with the carboxylic acid group and the amino group a further functional group selected form a carboxylic acid group and an amino group can be an amino acid unit selected from a 2,3-diaminopropionic acid (Dap) unit, 2,4-diaminobutanoic acid (Dab) unit, ornithine (Orn) unit and a lysine (Lys) unit, most preferably a Dap unit. In terms of their stereochemistry, the amino acids from which these units are derived are preferably in D-configuration.

For example, the trivalent amino acid unit comprising a —N(R)₂+- group in line with (ii) above can be derived from N-dialkylated 2,3-diaminopropionic acid (Dap), N-di dialkylated 2,4-diaminobutanoic acid (Dab), N-dialkylated ornithine (Orn) and N-dialkylated lysine (Lys).

In line with the above, a preferred structure of the trivalent linking unit L^{T} can be illustrated by the following formula (L-3):

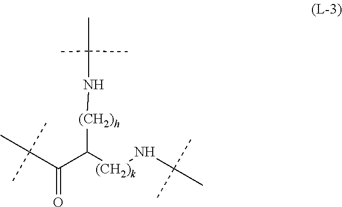

wherein either h is 0 and k is an integer of 1 to 4, more preferably 1, or k is 0 and h is an integer of 1 to 4, more preferably 1, wherein the dashed lines mark bonds attached to adjacent atoms or units, and wherein the bond marked by the dashed line at the carbonyl group —C(O)—is formed with L^{D}.

The hydrophilic modifying group -R^{H} comprises one or more hydrophilic units selected from a carbohydrate unit, a polyvalent alcohol unit, a polyvalent carboxylic acid unit and an amino acid unit derived from a hydrophilic amino acid which comprises a further hydrophilic functional group in addition to its —NH₂ and its —COOH functional group.

The group R^{H} shown in the above formulae (R-3b), (R-3d) and (IE) is a hydrophilic modifying group, i.e. a group which enhances the hydrophilic characteristics of the compounds in accordance with the invention.

Preferably, the hydrophilic modifying group -R^{H} is a group of formula (H-1):

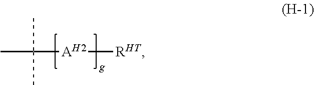

wherein
g is an integer of 0 to 5, preferably 1 to 3, still more preferably 2 or 3
A^{H2} is, independently for each occurrence if g is more than 1, an amino acid unit derived from a hydrophilic amino acid which comprises a further hydrophilic functional group in addition to its —NH₂ and its —COOH functional group,

R^{HT} is selected from a terminal hydrogen atom attached to an amino acid unit A^{H2}, an acetyl group or a hydrophilic unit selected from a carbohydrate group, a polyvalent alcohol unit and a polyvalent carboxylic acid unit, and the dashed line marks a bond which attaches the group to the remainder of the compound. Thus, as will be understood by the skilled person, the bond marked with the dashed line does not carry a methyl group opposite to A^{H2}, but rather represents a covalent bond which attaches R^{H} to L^{T} in the above formulae.

As will be understood from the above, if g is 1 or more, R^{HT} can be any of a terminal hydrogen atom, an acetyl group, or a hydrophilic unit selected from a carbohydrate group, a polyvalent alcohol unit (e.g. provided by an acyl group derived from quinic acid) and a polyvalent carboxylic acid unit. If g is 0, R^{HT} is preferably a hydrophilic unit selected from a carbohydrate group, a polyvalent alcohol unit and a polyvalent carboxylic acid unit.

A^{H2} is an amino acid unit, i.e. a group which can be derived from an amino acid. Unless indicated otherwise in a specific context, the amino acids from which the amino acid units can be derived are preferably α-amino acids. If an amino acid unit can be derived from a chiral amino acid, preference is given to the D-configuration.

As will be further understood, an amino acid unit can be derived from an amino acid by using one or more of its functional groups to provide a coupling group which forms a bond to an adjacent atom or group to which the amino acid unit is attached. For example, an amino group of the amino acid may be used to provide a coupling group —NH—wherein the bond to one hydrogen atom in the amino group is replaced by a bond to another adjacent atom or group. A carboxylic acid group of the amino acid may be used to provide a coupling group —C(O)—wherein the bond to the —OH group is replaced by a bond to another adjacent atom or group. Preferably, any coupling group provided by the amino acid is covalently linked to a further, complementary coupling group in the compound in accordance with the invention, so that the two complementary coupling groups combine to form a binding unit, such as an amide bond (—C(O)—NH—) or an alkylated amide bond —C(O)—NR—, preferably an amide bond. R is C1 to C6 alkyl, preferably methyl.

Specifically in formula (H-1), A^{H2} is, independently for each occurrence if g is more than 1, an amino acid unit derived from a hydrophilic amino acid which comprises, in addition to its —NH₂ and its —COOH functional group, a further hydrophilic functional group.

For example, the further hydrophilic functional group of the amino acid unit(s) A^{H2} can be selected, independently for each occurrence if g is more than 1, from —NH₂, —COOH, —NH—C(═NH)—NH₂, —C(═O) NH₂, —NH—C(═O)—NH₂, —OH and —P(═O)(OH)₂.

Preferably, each of the g amino acid unit(s) A^{H2} comprises, independently for each occurrence if f is more than 1, a side chain having a terminal hydrophilic functional group which side chain is selected from —(CH₂)ᵥ—NH₂, —(CH₂)ᵥ—COOH, —(CH₂)ᵥ—NH—C(═NH)—NH₂, —(CH₂)ᵥ—C(═O) NH₂, —(CH₂)ᵥ—NH—C(═O)—NH₂, —(CH₂)ᵥ—OH and —(CH₂)ᵥ—P(═O)(OH)₂ wherein v is 1 to 4.

Thus, it is preferred that the amino acid unit(s) A^{H2} is (are) selected, independently for each occurrence if g is more than 1, from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, serine (Ser) unit, citrulline (Cit) unit, thiocitrullin unit, methylisothiocitrulline unit, canavanin unit, thiocanavanin unit, α-amino-γ-(thioureaoxy)-n-butyric acid unit, α-amino-γ-(thioureathia)-n-butyric acid unit, and a phosphonomethylalanine (Pma) unit. They are preferably units which can be derived from amino acids in D-configuration. Particularly preferred are units (is a unit) selected from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, serine (Ser) unit, a citrulline (Cit) unit and a phosphonomethylalanine (Pma) unit. Thus, for example, a preferred group [A^{H2}], wherein f is 1 may be provided by an Asp unit or by a Glu unit.

Preferably, the group -[A^{H2}]_{g}-provides a C-terminus which forms an amide bond with the NH group to which the group -[A^{H2}]_{g}- is attached in formula (H-1), and an N-terminus which forms an amide bond with L^{T} or R^{S}, respectively.

In line with the above, the group -R^{H} is preferably a group of the formula:

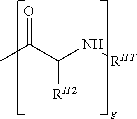

wherein g is as defined above. Each of the g groups R^{H2}, independently for each occurrence if g is more than 1, is selected from
—(CH₂)ᵥ—NH₂, —(CH₂)ᵥ—COOH, —(CH₂)ᵥ—NH—C(═NH)—NH₂, —(CH₂)ᵥ—C(═O) NH₂, —(CH₂)ᵥ—NH—C(═O)—NH₂, —(CH₂)ᵥ—OH and —(CH₂)ᵥ—P(═O)(OH)₂ wherein v is 1 to 4.

It is preferred that the amino acid unit(s)—C(O)—CH(R^{H2})—NH—of the above formula is (are) selected, independently for each occurrence if g is more than 1, from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, serine (Ser) unit, a citrulline (Cit) unit and a phosphonomethylalanine (Pma) unit. They are preferably units which can be derived from amino acids in D-configuration. Thus, for example, a preferred group —[C(O)—CH(R^{H2})—NH], —may be provided by 3 hydrophilic amino acid units comprising two Glu units or two Cit units, and a third unit selected from a Cit unit, a Glu unit, a Dap unit, and a Lys unit.

Further in line with the above, particularly preferred compounds of formula (I) can be illustrated by the following formulae (IF) and (IG).

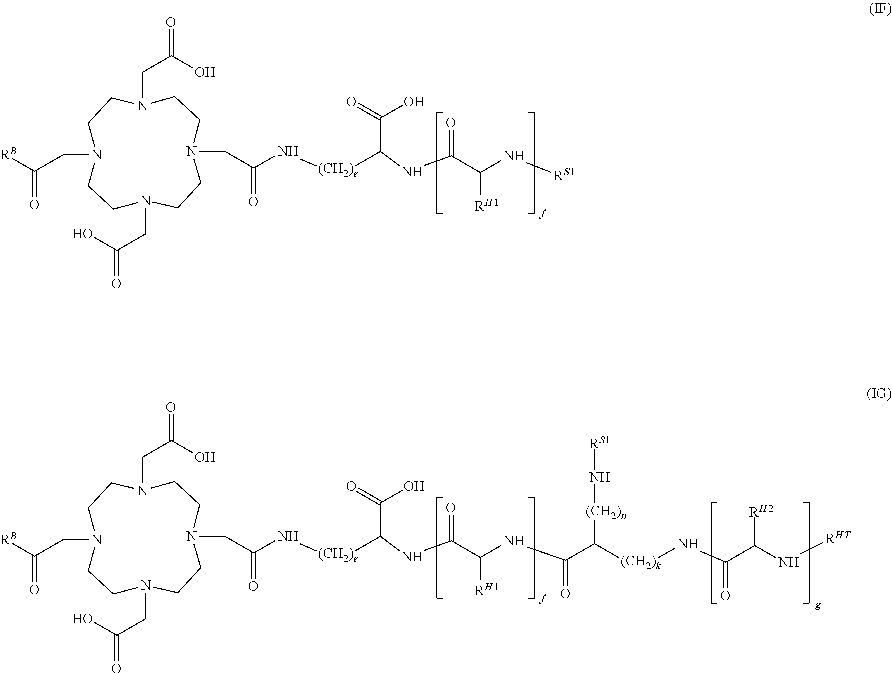

wherein the variables have the meanings as defined herein, including any preferred embodiments thereof, and R^{S1} is a SiFA group of formula (S-3) as defined herein, preferably of formula (S-4) as defined herein.

As noted above, the compounds in accordance with the invention encompass the compounds of formula (I), their salts, and chelate compounds formed from the compounds of formula (I) or their salts and a radioactive or non-radioactive cation. Salts are preferably pharmaceutically acceptable salts, i.e. formed with pharmaceutically acceptable anions or cations. Salts may be formed, e.g., by protonation of an atom carrying an electron lone pair which is susceptible to protonation, such as a nitrogen atom, with an inorganic or organic acid, or by separating a proton from an acidic group, such as a carboxylic acid group, e.g. by neutralization with a base. Other charged groups which may be present in the compounds in accordance with the invention and which may provide the compounds in the form of a salt include groups which are continuously charged, such as a quaternary ammonium group comprising an ammonium cation wherein the nitrogen is substituted by four organyl groups, or charged chelate complexes.

As exemplary anions which may be present as counterions in salt forms of the compounds of the invention, mention may be made, for example, of an anion selected from chloride, bromide, iodide, sulfate, nitrate, phosphate (such as, e.g., phosphate, hydrogenphosphate, or dihydrogenphosphate salts), carbonate, hydrogencarbonate or perchlorate; acetate, trifluoroacetate, propionate, butyrate, pentanoate, hexanoate, heptanoate, octanoate, cyclopentanepropionate, undecanoate, lactate, maleate, oxalate, fumarate, tartrate, malate, citrate, nicotinate, benzoate, salicylate or ascorbate; sulfonates such as methanesulfonate, ethanesulfonate, 2-hydroxyethanesulfonate, benzenesulfonate, p-toluenesulfonate (tosylate), 2-naphthalenesulfonate, 3-phenylsulfonate, or camphorsulfonate. As illustrated in the examples provided in the application, trifluoroacetic acid can be used during the synthesis of the compounds in accordance with the invention, so that trifluoroacetate salts can be conveniently provided, or may be conveniently converted to acetate salts if desired, such that trifluoroactate salts and acetate salts may be mentioned as preferred salt forms.

As exemplary cations which may be present as counterions in salt forms of the compounds of the invention if the salt form comprises a negatively charged form of the compound of formula (I) or (II), mention may be made, for example, of a cation selected from alkali metal cations, such as lithium, sodium or potassium, alkaline earth metal cations, such as calcium or magnesium; and ammonium (including ammonium ions substituted by organic groups).

As noted above, the compounds of the invention also include chelate compounds which are formed from a compound of formula (I) or its salt, and a radioactive or non-radioactive cation. As illustrated by formula (I) (or by the preferred embodiments thereof, such as (IA) to (IF)), the compounds of the invention comprise a substituted nitrogen containing heterocycle, and it will be appreciated by the skilled reader that the substituted nitrogen containing heterocycle can suitably provide a chelating ligand for a cation. Thus, in the compounds of the invention, a chelate compound can be conveniently obtained by providing a chelate ligand using the substituted nitrogen containing heterocycle comprised in formula (I) (or in the preferred embodiments thereof, such as (IA) to (IF)). The chelate compound comprises the radioactive or non-radioactive cation as a chelated cation. As will be understood, the chelate ligand acts as a ligand for the radioactive or non-radioactive cation in the chelate compound.

Since the compounds of the invention comprise a substituted nitrogen containing heterocycle suitable as a chelating ligand as a bridging group between an effector moiety R^{B} (or a group comprising such a moiety, respectively) and a SiFA moiety R^{S} (or a group comprising such a moiety, respectively), the compounds of the invention can be considered as compounds comprising a chelating group as a bridging group.

As exemplary radioactive or non-radioactive cations which may be comprised as chelated cations by such a chelate compound, reference can be made to cations of ⁴³Sc, ⁴⁴Sc, ⁴⁷Sc, ⁵¹Cr, ^{52m}Mn, ⁵⁵Co, ⁵⁷Co, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁸Ga, ⁶⁷Ga, ⁸⁹Zr, ⁹⁰Y, ⁸⁶Y, ^{94m}mTc, ^{99m}Tc, ⁹⁷Ru, ¹⁰⁵Rh, ¹⁰⁹Pd, ¹¹Ag, ^{110m}In, ¹¹¹ In, ^{113m}In, ^{114m}In, ^{117m}Sn, ¹²¹Sn, ¹²⁷Te, ¹⁴²Pr, ¹⁴³Pr, ¹⁴⁷Nd, ¹⁴⁹Gd, ¹⁴⁹Pm, ¹⁵¹Pm, ¹⁴⁹Tb, ¹⁵²Tb, ¹⁵⁵Tb, ¹⁵³Sm, ¹⁵⁶Eu, ¹⁵⁷Gd, ¹⁵⁵Tb, ¹⁶¹Tb, ¹⁶⁴Tb, ¹⁶¹Ho, ¹⁶⁶Ho, ¹⁵⁷Dy, ¹⁶⁵Dy, ¹⁶⁶Dy, ¹⁶⁰Er, ¹⁶⁵Er, ¹⁶⁹Er, ¹⁷¹Er, ¹⁶⁶Yb, ¹⁶⁹Yb, ¹⁷⁵Yb, ¹⁶⁷Tm, ¹⁷²Tm, ¹⁷⁷Lu, ¹⁸⁶Re, ^{186g}Re, ¹⁸⁸Re, ¹⁸⁸W, ¹⁹¹ Pt, ^{195m}Pt, ¹⁹⁴Ir, ¹⁹⁷Hg, ¹⁹⁸Au, ¹⁹⁹ Au, ²¹²Pb, ²⁰³Pb, ²¹¹At, ²¹²Bi, ²¹³Bi, ²²³Ra, ²²⁴Ra, ²²⁵Ac, ²²⁶Th and ²²⁷Th, to cations of non-radioactive isotopes of these metals, or to a cationic molecule comprising ¹⁸F or ¹⁹F, such as ¹⁸F-[AIF]²⁺.

Preferably, the radioactive or non-radioactive cation is a cation of Lu, such as a cation of ¹⁷⁷Lu or of a non-radioactive isotope of Lu, a cation of Y, such as a cation of ⁹⁰Y or of a non-radioactive isotope of Y, or a cation of Ga, such as a cation of ⁶⁸Ga or of a non-radioactive isotope of Ga. Particularly preferred is a cation of Ga, such as a cation of ⁶⁸Ga or of a non-radioactive isotope of Ga.

The compounds in accordance with the invention preferably exhibit an octanol-water distribution coefficient (also referred to as logD_{7.4} or logP value), of −1.0 or less, more preferably −2.0 or less. It is generally not below −4.0.

This distribution coefficient may be determined by measuring the equilibrium distribution, e.g. at room temperature (20° C.ₒ) of a compound in accordance with the invention in a two-phase system containing equal amounts, such as 1.00 ml each, of n-octanol and PBS (pH=7.4), and calculating the logD_{7.4} value as log 10 (concentration in octanol/concentration in PBS). Instead of the (absolute) concentration of the compound in accordance with the invention in the octanol and the PBS, a parameter which is proportional to the concentration of the compound in each phase may also be used for the calculation, such as the activity of radiation if the compound comprises a radioactive moiety, e.g. a radioactive chelate.

The compounds of the invention can provide advantageous binding characteristics to human serum albumin (HSA). Moderate to low HSA binding values, expressed as the apparent molecular weight in kDa and determined via radio inversed affinity chromatography (RIAC) as described in the examples section below can be achieved. Preferably, the HSA binding value is less than 22 kDa, more preferably below 10 kDa.

As exemplary compounds in accordance with the invention, the following are further mentioned.

Ligand compound 01 having the formula shown in the Examples section below or a salt thereof, or a chelate compound formed from the ligand compound or its salt and a radioactive or non-radioactive cation.

Ligand compound 02 having the formula shown in the Examples section below or a salt thereof, or a chelate compound formed from the ligand compound or its salt and a radioactive or non-radioactive cation.

Ligand compound 03 having the formula shown in the Examples section below or a salt thereof, or a chelate compound formed from the ligand compound or its salt and a radioactive or non-radioactive cation.

Ligand compound 04 having the formula shown in the Examples section below or a salt thereof, or a chelate compound formed from the ligand compound or its salt and a radioactive or non-radioactive cation.

Ligand compound 05 having the formula shown in the Examples section below or a salt thereof, or a chelate compound formed from the ligand compound or its salt and a radioactive or non-radioactive cation.

Ligand compound 06 having the formula shown in the Examples section below or a salt thereof, or a chelate compound formed from the ligand compound or its salt and a radioactive or non-radioactive cation.

Ligand compound 07 having the formula shown in the Examples section below or a salt thereof, or a chelate compound formed from the ligand compound or its salt and a radioactive or non-radioactive cation.

Ligand compound 08 having the formula shown in the Examples section below or a salt thereof, or a chelate compound formed from the ligand compound or its salt and a radioactive or non-radioactive cation.

Ligand compound 09 having the formula shown in the Examples section below or a salt thereof, or a chelate compound formed from the ligand compound or its salt and a radioactive or non-radioactive cation.

As exemplary radioactive or non-radioactive cation chelated in exemplary chelate compounds formed from ligand compounds 01 to 09 or their salts, respectively, cations of Ga, such as a cation of ⁶⁸Ga or a cation of a non-radioactive isotope of Ga, and cations of Lu, such as a cation of ¹⁷⁷Lu or a cation of a non-radioactive isotope of Lu can be mentioned.

In a further aspect, the present invention provides a pharmaceutical composition (also referred to as a therapeutic composition) comprising or consisting of one or more types, preferably one type, of the compound in accordance with the invention. As noted above, the compound may be a compound of formula (I) or its preferred embodiments disclosed herein, a salt of a compound of formula (I) or its preferred embodiments, or a chelate compound formed from the compound of formula (I) or its preferred embodiments or from a salt thereof. In a related aspect, the compound in accordance with the invention is provided for use in therapy or for use as a medicament. Thus, the compound of the invention can be used in a therapeutic method, which method may comprise administering the ligand compound to a subject. The subject may be a human or an animal and is preferably a human. Preferably, the compound of the invention is provided for use in a method of treatment of the human or animal body by therapy, wherein the therapy is radionuclide therapy.

The therapy or therapeutic method referred to above aims at the treatment or prevention of a disease or disorder of the human or animal body, e.g. cancer.

In cases where the effector moiety R^{B} comprised by formula (I) is a binding motif which is able to bind to a somatostatin receptor, the disease or disorder may be a disease or disorder that is associated with increased or aberrant expression of a somatostatin receptor. For example, such a disease or disorder may be a tumor which overexpresses at least one of SST₁ to SST₅, such as SST₂. For example, such a tumor may be a neuroendocrine tumor.

For example, a compound in accordance with the invention which is a chelate compound comprising a chelated radioactive cation, such as a ¹⁷⁷Lu cation, or a ⁹⁰Y cation, can be advantageously used in radionuclide therapy, such as the radionuclide therapy of a disease or disorder as discussed above.

In another aspect, the present invention provides a diagnostic composition comprising or consisting of one or more types, preferably one type, of the compound in accordance with the invention. As noted above, the compound may be a compound of formula (I) or its preferred embodiments disclosed herein, a salt of a compound of formula (I) or its preferred embodiments, or a chelate compound formed from the compound of formula (I) or its preferred embodiments or from a salt thereof. In a related aspect, the compound in accordance with the invention is provided for use in a method of diagnosis in vivo of a disease or disorder. Thus, the compound in accordance with the invention can be used in a method of diagnosis, which method may comprise administering the ligand compound to a subject and detecting the compound in the subject, or monitoring the distribution of the compound in the subject thereby detecting or monitoring the disease to be diagnosed. For example, nuclear imaging, e.g. by means of Positron Emission Tomography (PET) or Single Photon Emission Computed Tomography (SPECT), respectively, can be used for detecting or monitoring a compound in accordance with the invention. The subject may be a human or an animal and is preferably human. Alternatively, a method of diagnosis may also comprise adding the compound to a sample, e.g. a physiological sample obtained from a subject in vitro or ex vivo, and detecting the compound in the sample.

The method of diagnosis referred to above aims at the identification of a disease or disorder of the human or animal body, such as cancer. Thus, in terms of a diagnostic application, the compounds of the invention are preferably provided for use in a method of diagnosis in vivo of cancer.

In cases where the effector moiety R^{B} comprised by formula (I) is a binding motif which is able to bind to a somatostatin receptor, the disease or disorder may be a disease or disorder that is associated with increased or aberrant expression of a somatostatin receptor. For example, such a disease or disorder may be a tumor which overexpresses at least one of SST₁ to SST₅, such as SST₂. For example, such a tumor may be a neuroendocrine tumor.

For example, a compound of the invention wherein the SiFA group comprises a ¹⁸F fluoride, or a compound of the invention is a chelate compound comprising a chelated radioactive cation, e.g. a ⁶⁸Ga cation, can be advantageously used for nuclear diagnostic imaging, such as diagnosis via positron emission tomography (PET) or via Single Photon Emission Computed Tomography (SPECT).

It will be understood that suitability for a therapeutic and a diagnostic application is not mutually exclusive. i.e. a compound in accordance with the invention may be suitable for both applications. For example, a compound comprising a chelated ¹⁷⁷Lu cation can be used both for therapeutic and diagnostic imaging applications. Moreover, due to the presence of a chelating group and a SiFA group, the compounds of the invention are suitable as radiohybrid (rh) ligands. Such a rh ligand can be alternatively labeled with [¹⁸F]fluoride (e.g. for PET) or a radiometal (such as a ⁶⁸Ga cation for PET, or a ¹⁷⁷Lu cation for radiotherapy). When a rh ligand is labeled with [¹⁸F]fluoride, a cold (non-radioactive) metal cation can, but not necessarily must be complexed elsewhere in the molecule, and when it is labeled with a corresponding radioactive metal cation, cold [¹⁹F] fluorine can be included. Therefore, the ¹⁸F-labeled compound and the corresponding radiometal-labeled analog can possess the same chemical structure and thus identical in vitro and in vivo properties, thereby allowing the generation of structurally identical theranostic tracers with exactly the same in vivo properties of the diagnostic and therapeutic tracers (e.g. ¹⁸F/¹⁷⁷Lu analogs) [20].

Thus, in line with this approach the compounds of the invention include compounds wherein the silicon-based fluoride acceptor group is labeled with ¹⁸F and the chelating group contains a chelated non-radioactive cation (such as ^{nat}Lu or ^{nat}Ga), and compounds wherein the chelating group contains a chelated radioactive cation (such as ¹⁷⁷Lu or ⁶⁸Ga) and the silicon-based fluoride acceptor group is not labeled with ¹⁸F (thus carrying a ¹⁹F). Likewise, the invention provides the compounds of the invention for use in a hybrid method of diagnosis in vivo and therapy of a disease or disorder associated with increased or aberrant expression of a somatostatin receptor as discussed above, wherein the method involves first the administration of a compound of the invention wherein the silicon-fluoride acceptor group is labeled with ¹⁸F and the chelating group contains a chelated non-radioactive cation (such as ^{nat}Lu or ^{nat}Ga), and subsequently of a compound wherein the chelating group contains a chelated radioactive cation and the silicon-fluoride acceptor group is not labeled with ¹⁸F.

Thus, in another aspect, the present invention provides a dedicated composition comprising or consisting of one or more types, preferably one type, of the compound in accordance with the invention for use in a method of in vivo imaging of a disease or disorder. As noted above, the compound may be a compound of formula (I) or its preferred embodiments disclosed herein, a salt of a compound of formula (I) or its preferred embodiments, or a chelate compound formed from the compound of formula (I) or its preferred embodiments or from a salt thereof. The compound in accordance with the invention can be used in an imaging method, which method may comprise administering the ligand compound to a subject and detecting the ligand compound in the subject and monitoring the distribution of the ligand compound in vivo at different time points after injection with the aim to calculate the dosimetry prior or during a therapeutic treatment. The subject may be a human or an animal and is preferably human. The imaging method may be used for the calculation of the dosimetry prior or during a therapeutic treatment of a disease or disorder of the human or animal body, such as cancer. In cases where the effector moiety R^{B} comprised by formula (I) is a binding motif which is able to bind to a somatostatin receptor, the disease or disorder may be a disease or disorder that is associated with increased or aberrant expression of a somatostatin receptor. For example, such a disease or disorder may be a tumor which overexpresses at least one of SST₁ to SST₅, such as SST₂. For example, such a tumor may be a neuroendocrine tumor.

For example, a compound of the invention wherein the SiFA group comprises a ¹⁸F fluoride and non-radioactive ^{nat}Lu, or a compound of the invention wherein the chelating group comprises a chelated radioactive cation, e.g. a ¹⁷⁷Lu cation, whereas the SiFA is non-radioactive, can be advantageously used for nuclear imaging by means of Positron Emission Tomography (PET) or Single Photon Emission Computed Tomography (SPECT), respectively, to monitor the distribution of the applied compound and thereafter calculate the individual dosimetry by means of the quantitative distribution kinetics.

The pharmaceutical or diagnostic composition may further comprise one or more pharmaceutically acceptable carriers, excipients and/or diluents. Examples of suitable pharmaceutical carriers, excipients and/or diluents are well known in the art and include phosphate buffered saline solutions, amino acid buffered solutions (with or without saline), water for injection, emulsions, such as oil/water emulsions, various types of wetting agents, sterile solutions etc. Compositions comprising such carriers can be formulated by well-known conventional methods. These compositions can be administered to the subject at a suitable dose. Administration of the suitable compositions may be accomplished in different ways, e.g., by intravenous, intraperitoneal, subcutaneous, intramuscular, topical, intradermal, intranasal or intrabronchial administration. It is particularly preferred that said administration is carried out by intravenous injection and/or delivery. The compositions may be administered directly to the target site. The dosage regimen will be determined by the attending physician and clinical factors. As is well known in the medical arts, dosages for any one patient depends upon many factors, including the patient's size, body surface area, age, the particular compound to be administered, dosimetry, sex, time and route of administration, general health, and other drugs being administered concurrently. The compounds may be administered e.g. in amounts between 0.1 ng and 10 μg/kg body weight. For example, in diagnostic applications, a typical dosage amount of the compounds of the invention or their salts is <100 μg/patient, e.g. in the range of 0.1 to 30 μg/patient, however, if appropriate, higher or lower dosages can be envisaged. A typical dosage amount of the compounds of the invention or their salts in a radiotherapeutic application is in the range of 50 to 200 μg/patient, preferably 75 to 150 μg/patient, however, if appropriate, higher or lower dosages can be envisaged.

The following items summarize aspects of the invention. It will be understood that these items are closely related to the above parts of the description, and that the information provided in these items may supplement the above parts of the description and vice versa.

1. A compound selected from:
(a) a compound of formula (I)

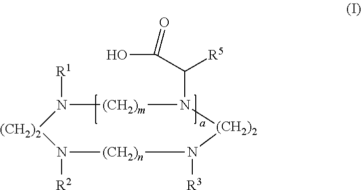

wherein
a is 0 or 1, preferably 1;
m is 2 or 3, preferably 2;
n is 2 or 3, preferably 2;
one group selected from R¹, R² and R³ is a group comprising an effector moiety R^{B}; another group selected from R¹, R² and R³ is a group comprising a silicon-based fluoride acceptor (SiFA) moiety R^{S}, which moiety comprises a silicon atom and a fluorine atom, wherein the fluorine atom is linked via a covalent bond directly to the silicon atom, and which can be labeled with ¹⁸F by isotopic exchange of ¹⁹F by ¹⁸F or which is labeled with ¹⁸F;
and the remaining group selected from R¹, R² and R³ is a group of the formula (R-1):

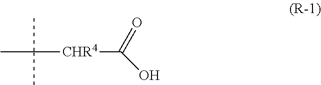

wherein
R⁴ is selected from —H, —OH and C1-C3 alkyl, and is preferably —H; and wherein the dashed line marks a bond which attaches the group to the remainder of the compound;
R⁵ is selected from —H, —OH and C1-C3 alkyl, and is preferably —H;
(b) a salt thereof, and
(c) a chelate compound formed from a compound of formula (I) or its salt, and a radioactive or non-radioactive cation.

2. The compound in accordance with item 1, wherein the SiFA moiety R^{S} comprises a group of formula (S-1):

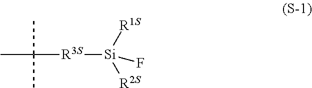

wherein
R^{1S} and R^{2S} are independently from each other a linear or branched C3 to C10 alkyl group, preferably R^{1S} and R^{2S} are selected from isopropyl and tert-butyl, and more preferably R^{1S} and R^{2S} are tert-butyl; and
R^{3S} is a divalent C1 to C20 hydrocarbon group which comprises one or more aromatic and/or aliphatic moieties, and which optionally comprises up to 3 heteroatoms selected from O and S, preferably R^{3S} is a divalent C6 to C12 hydrocarbon group which comprises an aromatic ring and which may comprise one or more aliphatic moieties; and wherein the dashed line marks a bond which attaches the group to the remainder of the compound.

3. The compound in accordance with item 1 or 2, wherein the SiFA moiety R^{S} comprises a group of the formula (S-2):

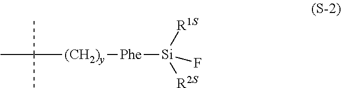

wherein
R^{1S} and R^{2S} are independently from each other a linear or branched C3 to C10 alkyl group, preferably R^{1S} and R^{2S} are selected from isopropyl and tert-butyl, and more preferably R^{1S} and R^{2S} are tert-butyl, Phe is a phenylene group, y is an integer of 0 to 6 and is preferably 1, and wherein the dashed line marks a bond which attaches the group to the remainder of the compound.

4. The compound in accordance with any of items 1 to 3, wherein the SiFA moiety R^{S} is a group of the formula (S-3):

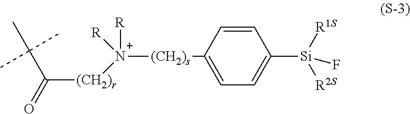

wherein
r is 1, 2 or 3, preferably 1, s is an integer of 1 to 6 and is preferably 1,
R is, independently, C1 to C6 alkyl and is preferably methyl, and
R^{1S} and R^{2S} are independently from each other a linear or branched C3 to C10 alkyl group, preferably R^{1S} and R^{2S} are selected from isopropyl and tert-butyl, and more preferably R^{1S} and R^{2S} are tert-butyl; and wherein the dashed line marks a bond which attaches the group to the remainder of the compound.

5. The compound in accordance with any of items 1 to 4, wherein the SiFA moiety R^{S} is a group of the formula (S-4):

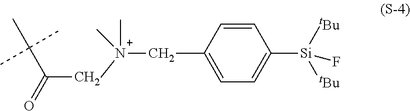

wherein ^{t}Bu indicates a tert-butyl group and the dashed line marks a bond which attaches the group to the remainder of the compound.

6. The compound in accordance with any of items 1 to 5, wherein the effector moiety R^{B} is a peptidic binding motif which is able to bind to a receptor.

7. The compound in accordance with item 6, wherein R^{B} is a peptidic binding motif which is able to bind to a somatostatin receptor, preferably to the somatostatin receptor 2 (SST₂).

8. The compound in accordance with item 7, wherein R^{B} is a moiety which can be derived from a receptor agonist or receptor antagonist selected from Tyr³-Octreotate (TATE, H-D-Phe-cyclo(L-Cys-L-Tyr-D-Trp-L-Lys-L-Thr-L-Cys)-L-Thr-OH), Thr⁸-Octreotide (ATE), Phe1-Tyr³-Octreotide (TOC, H-D-Phe-cyclo(L-Cys-L-Tyr-D-Trp-L-Lys-L-Thr-L-Cys)-L-Thr-ol), NaI³-Octreotide (NOC, H-D-Phe-cyclo(L-Cys-L-1-Nal-D-Trp-L-Lys-L-Thr-L-Cys)-L-Thr-ol), 1-NaI³,Thr⁸-Octreotide (NOCATE), BzThi3-Octreotide (BOC), BzThi3, Thr⁸-Octreotide (BOCATE), JR11 (H-L-Cpa-cyclo(D-Cys-L-Aph(Hor)-D-Aph (Cbm)-L-Lys-L-Thr-L-Cys)-D-Tyr-NH₂), BASS (H-L-Phe (4-NO₂)-cyclo(D-Cys-L-Tyr-D-Trp-L-Lys-L-Thr-L-Cys)-D-Tyr-NH₂) and KE121 (cyclo(D-Dab-L-Arg-L-Phe-L-Phe-D-Trp-L-Lys-L-Thr-L-Phe).

9. The compound of item 8, wherein R^{B} is a group of the formula (B-1):

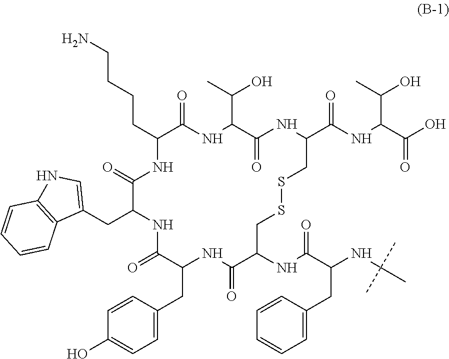

wherein the dashed line marks a bond which attaches the group to the remainder of the compound.

10. The compound in accordance with any of items 1 to 9, wherein the compound of formula (I) is a compound of formula (IB):

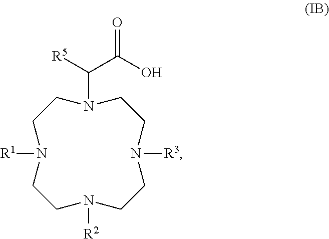

wherein R¹, R², R³ and R⁵ are defined as in any of the preceding items.

11. The compound in accordance with any of items 1 to 10, wherein the group comprising an effector moiety R^{B} is a group of the formula (R-2a) or (R-2b), preferably of the formula (R-2a):

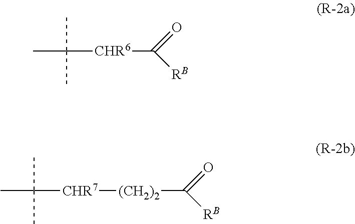

wherein
R^{B} is as defined in any one of the preceding items;
R⁶ is selected from —H, —OH and C1-C3 alkyl, and is preferably —H; and R⁷ is —COOH;
and wherein the dashed line marks a bond which attaches the group to the remainder of the compound.

12. The compound in accordance with any of items 1 to 11, wherein the group comprising the SiFA moiety R^{S} is a group of the formula (R-3a), (R-3b), (R-3c) or (R-3d), preferably of the formula (R-3a) or (R-3b).

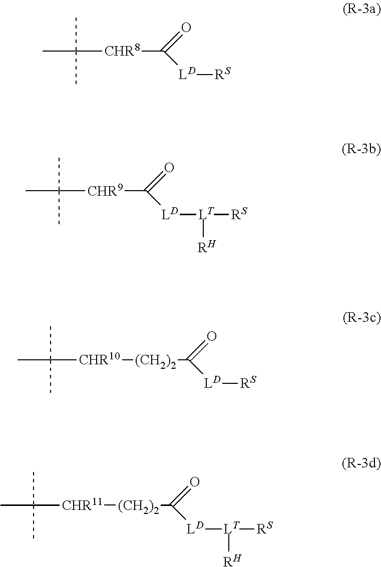

wherein
R^{S} is as defined in any one of the preceding items; R⁸ and R⁹ are selected from —H, —OH and C1-C3 alkyl, and are preferably —H;
R¹⁰ and R¹¹ are —COOH;
L^{D} is a divalent linking group;
L^{T} is a trivalent linking group;
R^{H} is a hydrophilic modifying group;
and the dashed line marks a bond which attaches the group to the remainder of the compound.

13. The compound in accordance with any of items 11 or 12, wherein the compound of formula (1) is a compound of formula (IC):

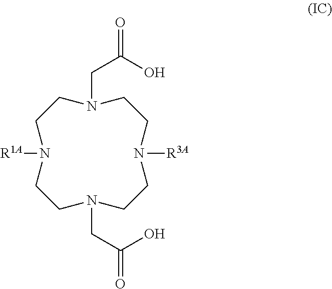

wherein
i) R^{1A} is a group of formula (R-2a) as defined in item 11 and R^{3A} is selected from the groups of formula (R-3a), (R-3b), (R-3c) and (R-3d) as defined in item 12; or
ii) R^{1A} is selected from the groups of formula (R-2a) and (R-2b) as defined in item 11 and R^{3A} is selected from the groups of formula (R-3a) and (R-3b) as defined in item 12.

14. The compound in accordance with any of items 1 to 13, wherein the compound of formula (I) is a compound of formula (ID) or (IE):

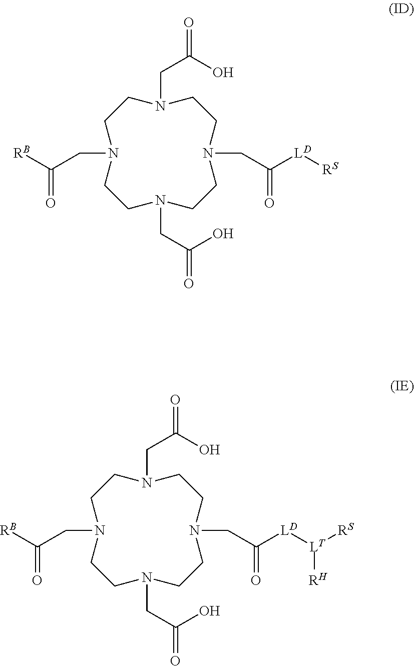

wherein
R^{B} and R^{S} are as defined in any one of the preceding items;
L^{D} is a divalent linking group;
L^{T} is a trivalent linking group; and
R^{H} is a hydrophilic modifying group.

15. The compound in accordance with any of items 12 to 14, wherein the divalent linking group L^{D} comprises an—NH—group at each of its two termini for attachment to adjacent groups.

16. The compound in accordance with any of items 12 to 15, wherein the divalent linking group L^{D} comprises or consists of a group (L-1):

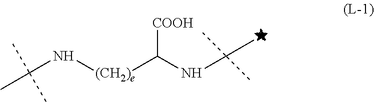

wherein e is an integer of 1 to 6, preferably 1 to 4, the dashed lines mark bonds which attach the group to adjacent groups, and the bond additionally marked by the asterisk is preferably attached to R^{S} or L^{T}, respectively.

17. The compound in accordance with any of items 12 to 16, wherein the divalent linking group L^{D} comprises one or more hydrophilic units selected from a hydrocarbon unit, a polyvalent alcohol unit, a polyvalent carboxylic acid unit and an amino acid unit derived from a hydrophilic amino acid which comprises a further hydrophilic functional group in addition to its —NH₂ and its —COOH functional group.

18. The compound in accordance with any of items 12 to 17, wherein the divalent linking group L^{D} is a group of formula (L-2):

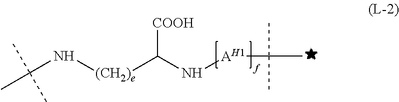

wherein
e is an integer of 1 to 6, preferably 1 to 4,
f is an integer of 0 to 5, preferably 0 or 1,
A^{H1} is, independently for each occurrence if f is more than 1, an amino acid unit derived from a hydrophilic amino acid which comprises a further hydrophilic functional group in addition to its —NH₂ and its —COOH functional group,
the dashed lines mark bonds which attach the group to adjacent groups, and the bond additionally marked by the asterisk is attached to R^{S} or RT, respectively.

19. The compound in accordance with item 17 or 18, wherein the hydrophilic amino acid unit is selected, independently for each occurrence if more than one of these units is present in L^{D}, from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, serine (Ser) unit, citrulline (Cit) unit and phosphonomethylalanine (Pma) unit.

20. The compound in accordance with any of items 12 to 19, wherein L^{T} is a trivalent amino acid unit.

21 The compound in accordance with item 20, wherein L^{T} is a trivalent amino acid unit selected from the following (i) and (ii), with (i) being preferred:
(i) a trivalent amino acid unit which can be derived from an amino acid comprising together with the carboxylic acid group and the amino group a further functional group selected form a carboxylic acid group and an amino group.
(ii) a trivalent amino acid unit comprising a —N(R)₂+- group which unit can be derived from a trifunctional amino acid comprising a tertiary amino group as a third functional group in addition to its —NH₂ group and its —COOH group, and wherein R is, independently, C1-C6 alkyl, preferably methyl.

22. The compound in accordance with item 21, wherein the trivalent amino acid unit which can be derived from an amino acid comprising together with the carboxylic acid group and the amino group a further functional group selected form a carboxylic acid group and an amino group is an amino acid unit selected from a 2,3-diaminopropionic acid (Dap) unit, 2,4-diaminobutanoic acid (Dab) unit, ornithine (Orn) unit and a lysine (Lys) unit, more preferably a Dap unit.

23. The compound in accordance with item 21, wherein the trivalent amino acid unit comprising a —N(R)₂+- group is derived from N-dialkylated 2,3-diaminopropionic acid (Dap), N-dialkylated 2,4-diaminobutanoic acid (Dab), N-dialkylated ornithine (Orn) and N-dialkylated lysine (Lys).

24. The compound in accordance with any of items 12 to 23, wherein the hydrophilic modifying group -R^{H} comprises one or more hydrophilic units selected from a hydrocarbon unit, a polyvalent alcohol unit, a polyvalent carboxylic acid unit and an amino acid unit derived from a hydrophilic amino acid which comprises a further hydrophilic functional group in addition to its —NH₂ and its —COOH functional group.

25. The compound in accordance with any of items 12 to 24, wherein the hydrophilic modifying group -R^{H} is a group of formula (H-1):

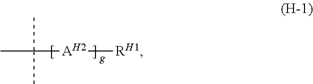

wherein
g is an integer of 0 to 5, preferably 1 to 3,
A^{H2} is, independently for each occurrence if g is more than 1, an amino acid unit derived from a hydrophilic amino acid which comprises a further hydrophilic functional group in addition to its —NH₂ and its —COOH functional group,
R^{H1} is selected from a terminal hydrogen atom attached to an amino acid unit A^{H2}, an acetyl group or a hydrophilic unit selected from a carbohydrate group, a polyvalent alcohol unit and a polyvalent carboxylic acid unit, and the dashed line marks a bond which attaches the group to the remainder of the compound.

26. The compound in accordance with item 25, wherein the hydrophilic amino acid unit A^{H2} is selected, independently for each occurrence if g is more than 1, from an from a 2,3-diaminopropionic acid (Dap) unit, 2,4-diaminobutanoic acid (Dab) unit, ornithine (Orn) unit, lysine (Lys) unit, arginine (Arg) unit, glutamic acid (Glu) unit, aspartic acid (Asp) unit, asparagine (Asn) unit, glutamine (Gln) unit, serine (Ser) unit, citrulline (Cit) unit and phosphonomethylalanine (Pma) unit.

27. The compound in accordance with any of items 1 to 26, wherein the radioactive or non-radioactive cation of the chelate compound is selected from the cations of ⁴³Sc, ⁴⁴Sc, ⁴⁷Sc, ⁵¹Cr, ^{52m}Mn, ⁵⁵Co, ⁵⁷Co, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁸Ga, ⁶⁷Ga, ⁸⁹Zr, ⁹⁰Y, ⁸⁶Y, ^{94m}Tc, ^{99m}Tc, ⁹⁷Ru, ¹⁰⁵Rh, ¹⁰⁹Pd, ¹¹¹ Ag, ^{110m}In, ¹¹¹ In, ^{113m} In, ^{14m}In, ^{117m}Sn, ¹²¹Sn, ¹²⁷Te, ¹⁴²Pr, ¹⁴³Pr, ¹⁴⁷Nd, ¹⁴⁹Gd, ¹⁴⁹Pm, ¹⁵¹Pm, ¹⁴⁹Tb, ¹⁵²Tb, ¹⁵⁵Tb, ¹⁵³Sm, ¹⁵⁶Eu, ¹⁵⁷Gd, ¹⁵⁵Tb, ¹⁶¹Tb, ¹⁶⁴Tb, ¹⁶¹Ho, ¹⁶⁶Ho, ¹⁵⁷Dy, ¹⁶⁵Dy, ¹⁶⁶Dy, ¹⁶⁰Er, ¹⁶⁵Er, ¹⁶⁹Er, ¹⁷¹Er, ¹⁶⁶Yb, ¹⁶⁹Yb, ¹⁷⁵Yb, ¹⁶⁷Tm, ¹⁷²Tm, ¹⁷⁷Lu, ¹⁸⁶Re, ^{186g}Re, ¹⁸⁸Re, ¹⁸⁸W, ¹⁹¹ Pt, ^{195m}Pt, ¹⁹⁴Ir, ¹⁹⁷Hg, ¹⁹⁸Au, ¹⁹⁹Au, ²¹²Pb, ²⁰³Pb, ²¹¹At, ²¹²Bi, ²¹³Bi, ²²³Ra, ²²⁴Ra, ²²⁵Ac, ²²⁶Th and ²²⁷Th, and from cations of non-radioactive isotopes thereof, or is a cationic molecule comprising ¹⁸F or ¹⁹F, such as ¹⁸F-[AIF] 2+, and is more preferably selected from a cation of ⁶⁸Ga, ⁹⁰Y, or ¹⁷⁷Lu and from cations of non-radioactive isotopes of Ga, Y or Lu.

28. The compound in accordance with any of items 1 to 27, which is a chelate compound comprising a chelated radioactive or non-radioactive gallium cation.

29. The compound in accordance with any of items 1 to 28, which is a chelate compound comprising a chelated radioactive cation, and wherein the SiFA group is not labeled with ¹⁸F.

30. The compound in accordance with item 29, wherein the chelated radioactive cation is a cation of ⁶⁸Ga.

31. The compound in accordance with any of items 1 to 28, which is a chelate compound comprising a chelated non-radioactive cation, or which is free of a chelated cation, and wherein the SiFA group is labeled with ¹⁸F.

32. A pharmaceutical composition comprising or consisting of one or more compounds in accordance with any of items 1 to 31.

33. The compound in accordance with any of items 1 to 31 for use as a medicament.

34 The compound in accordance with any of items 1 to 31 or the pharmaceutical composition in accordance with item 32 for use in a method of treatment of the human or animal body by therapy, wherein the therapy is radionuclide therapy.

35. The compound in accordance with any of items 1 to 31 or the pharmaceutical composition in accordance with item 32 for use in the treatment of cancer.

36 The compound or the pharmaceutical composition for use in accordance with item 35, wherein the cancer is a tumor which overexpresses at least one of SST₁ to SST₅.

37. A diagnostic composition comprising or consisting of one or more compounds in accordance with any of items 1 to 31.

38. The compound in accordance with any of items 1 to 31 or the diagnostic composition of item 36 for use in a method of diagnosing in vivo a disease or disorder.

39 The compound or the diagnostic composition for use of item 37, wherein the disease or disorder is cancer.

40. The compound or the diagnostic composition for use in accordance with item 38, wherein the cancer is a tumor which overexpresses at least one of SST₁ to SST₅.

41. The compound or salt or the diagnostic composition for use in accordance with any of items 37 to 39, wherein the method of diagnosing involves nuclear diagnostic imaging, wherein the nuclear diagnostic imaging is preferably positron emission tomography or single photon emission computerised tomography imaging.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### REFERENCES

1. Kaltsas G A, Besser G M, Grossman A B. The diagnosis and medical management of advanced neuroendocrine tumors [eng]. Endocr Rev. 2004; doi: 10.1210/er.2003-0014.
2. Modlin IM, Lye K D, Kidd M. A 5-decade analysis of 13,715 carcinoid tumors [eng]. Cancer. 2003; doi: 10.1002/cncr. 11105.
3. Taal B G, Visser O. Epidemiology of neuroendocrine tumours [eng].

Neuroendocrinology. 2004; doi: 10.1159/000080731.
4. Oronsky B, Ma P C, Morgensztern D, Carter C A. Nothing But NET: A Review of Neuroendocrine Tumors and Carcinomas [eng]. Neoplasia. 2017; doi: 10.1016/j.neo.2017.09.002.
5. Reubi J C, Waser B, Schaer J C, Laissue J A. Somatostatin receptor sst1-sst5 expression in normal and neoplastic human tissues using receptor autoradiography with subtype-selective ligands [eng]. Eur J Nucl Med. 2001; doi: 10.1007/s002590100541.
6. Xu C, Zhang H. Somatostatin receptor based imaging and radionuclide therapy [eng]. Biomed Res Int. 2015; doi: 10.1155/2015/917968.
7. Taniyama Y, Suzuki T, Mikami Y, Moriya T, Satomi S, Sasano H. Systemic distribution of somatostatin receptor subtypes in human: an immunohistochemical study [eng]. Endocr J. 2005; doi: 10.1507/endocrj.52.605.
8. Patel Y C. Somatostatin and its receptor family [eng]. Front Neuroendocrinol. 1999; doi: 10.1006/frne.1999.0183.
9. Goffin K. Al 18F-NOTA-octreotide and ¹⁸F-SiFAlin-TATE: two ‘new kids on the block’ in somatostatin receptor imaging [eng]. Eur J Nucl Med Mol Imaging. 2019; doi: 10.1007/s00259-019-04474-6.
10. Ilhan H, Todica A, Lindner S, Boening G, Gosewisch A, Wängler C, et al. First-in-human 18F-SiFAlin-TATE PET/CT for NET imaging and theranostics [eng]. Eur J Nucl Med Mol Imaging. 2019; doi: 10.1007/s00259-019-04448-8.
11. Litau S, Niedermoser S, Vogler N, Roscher M, Schirrmacher R, Fricker G, et al. Next Generation of SiFAlin-Based TATE Derivatives for PET Imaging of SSTR-Positive Tumors: Influence of Molecular Design on In Vitro SSTR Binding and In Vivo Pharmacokinetics [eng]. Bioconjug Chem. 2015; doi: 10.1021/acs.bioconjchem.5b00510.
12. Niedermoser S, Chin J, Wängler C, Kostikov A, Bernard-Gauthier V, Vogler N, et al. In Vivo Evaluation of ¹⁸F-SiFAlin-Modified TATE: A Potential Challenge for ⁶⁸Ga-DOTATATE, the Clinical Gold Standard for Somatostatin Receptor Imaging with PET [eng]. J Nucl Med. 2015; doi: 10.2967/jnumed. 114.149583.
13. Schottelius M, Wurzer A, Wissmiller K, Beck R, Koch M, Gorpas D, et al. Synthesis and Preclinical Characterization of the PSMA-Targeted Hybrid Tracer PSMA-I&F for Nuclear and Fluorescence Imaging of Prostate Cancer [eng]. J Nucl Med. 2019; doi: 10.2967/jnumed.118.212720.
14. Roxin Á, Zhang C, Huh S, Lepage M, Zhang Z, Lin K—S, et al. A Metal-Free DOTA-Conjugated ¹⁸F-Labeled Radiotracer: ¹⁸FDOTA-AMBF₃-LLP2A for Imaging VLA-4 Over-Expression in Murine Melanoma with Improved Tumor Uptake and Greatly Enhanced Renal Clearance [eng]. Bioconjug Chem. 2019; doi: 10.1021/acs.bioconjchem.9b00146.
15. Gai Y, Xiang G, Ma X, Hui W, Ouyang Q, Sun L, et al. Universal Molecular Scaffold for Facile Construction of Multivalent and Multimodal Imaging Probes [eng]. Bioconjug Chem. 2016; doi: 10.1021/acs.bioconjchem.6b00034.
16. Wurzer A, Vágner A, Horváth D, Fellegi F, Wester H-J, Kálmán FK, et al. Synthesis of Symmetrical Tetrameric Conjugates of the Radiolanthanide Chelator DOTPI for Application in Endoradiotherapy by Means of Click Chemistry [eng]. Frontiers in chemistry. 2018; doi: 10.3389/fchem.2018.00107.
17. Šimeček J, Hermann P, Havlíčková J, Herdtweck E, Kapp T G, Engelbogen N, et al. A cyclen-based tetraphosphinate chelator for the preparation of radiolabeled tetrameric bioconjugates [eng]. Chemistry. 2013; doi: 10.1002/chem.201300338.
18. Notni J, Šimeček J, Hermann P, Wester H-J. TRAP, a powerful and versatile framework for gallium-68 radiopharmaceuticals [eng]. Chemistry-A European Journal. 2011; doi: 10.1002/chem.201103503.
19. Poethko T, Schottelius M, Thumshirn G, Herz M, Haubner R, Henriksen G, et al. Chemoselective pre-conjugate radiohalogenation of unprotected mono- and multimeric peptides via oxime formation. Radiochimica Acta. 2004; doi: 10.1524/ract.92.4.317.35591.
20. Wurzer A, DiCarlo D, Schmidt A, Beck R, Eiber M, Schwaiger M, et al. Radiohybrid ligands: a novel tracer concept exemplified by ¹⁸F—or ⁶⁸Ga-labeled rhPSMA-inhibitors [eng]. J Nucl Med. 2019; doi: 10.2967/jnumed.119.234922.
21. Notni J, Pohle K, Wester H-J. Comparative gallium-68 labeling of TRAP-, NOTA-, and DOTA-peptides: practical consequences for the future of gallium-68-PET [eng]. EJNMMI Res. 2012; doi: 10.1186/2191-219X-2-28.
22. Wängler C, Niedermoser S, Chin J, Orchowski K, Schirrmacher E, Jurkschat K, et al. One-step (18) F-labeling of peptides for positron emission tomography imaging using the SiFA methodology [eng]. Nat Protoc. 2012; doi: 10.1038/nprot.2012.109.

### Abbreviations

2-CT 2-Chlorotrityl resin
2-CTC 2-Clorotrityl chloride resin
AA Amino acid
Acm Acetamidomethyl
Boc tert-Butyloxycarbonyl
BSA Bovine Serum Albumin
CHO Chinese hamster ovary
Cit Citrulline
Cys Cysteine
Dap 2,3-Diaminopropionic acid
DCM Dichloromethane
Dde N-(1-(4,4-dimethyl-2,6-dioxocyclohexylidene)ethyl)
DIC N,N′-Diisopropylcarbodiimide
DIPEA N,N-Diisopropylethylamine
DMEM/F12 Dulbecco's Modified Eagle
medium/Ham's F12 Nutrient
Mixture
DMF N,N-Dimethylformamide
DMSO Dimethylsulfoxide
DOTA((Bu)₂ trans-(Di-tert-butyl)-1,4,7,10-tetraazacycIododecane-1,4,7,10-tetraacetic acid
DOTA 1,4,7,10-tetraazacycIododecane-1,4,7,10-tetraacetic acid
EDTA Ethylenediaminetetraacetic acid
eq. Equivalents
ESI Electrospray ionization
FCS Fetal calf serum
Fmoc Fluorenylmethyloxycarbonyl
Glu Glutamic acid
Gly Glycine
GSP General synthetic procedure
HATU Hexafluorophosphate azabenzo-triazole tetramethyl uronium
HBSA HBSS with 1% BSA
HBSS Hank's Balanced Salt Solution
HFIP Hexafluoroisopropanol
HOAt 1-Hydroxy-7-azabenzotriazole
HPAC High performance affinity chromatography
HPLC High performance liquid chromatography
HSA Human serum albumin
IC₅₀ Half maximal inhibitory concentration
K′ Capacity factor
LogDP_{H=7.4} Logarithmic partition coefficient at pH=7.4
Lys Lysine
M Molar
M Molecular weight
m/z Mass-to-charge ratio
MS Mass spectrometry
NMP N-methyl-2-pyrrolidone
Oxyma Ethyl cyanohydroxyiminoacetate
p.i. Post injection
PBS Phosphate buffered saline
PEG Polyethylene glycol
PFP Pentafluorophenyl
PG Protecting group
Phe Phenylalanine
QC Quality control
RP Reversed-phase
rpm Revolutions per minute
RT Room temperature
SiFA Silicon-based fluoride acceptor
SiFA-Br (4-(bromomethyl)phenyl)di-tert-butylfluorosilane
SiFAlin- [¹⁸F] SiFA-GIC-L-Asp2-PEG1-TATE
TATE
SST Somatostatin
SST₂ Somatostatin transmembrane
receptor 2
TATE Tyr³-Octreotate
TBTU O-(Benzotriazol-1-yl)-N,N,N′,N′—tetramethyluroniumtetrafluoroborate
tBu tert-butyl
TFA Trifluoroacetic acid
THF Tetrahydrofuran
Thr Threonine
TIPS Triisopropylsilane
TLC Thin-layer chromatography
TOC Tyr³-Octreotide
t_{R} Retention time
Trp Tryptophane
TTFA Thallium (III) trifluoroacetate
Tyr Tyrosine
UV/VIS Ultraviolet/visible light

The following examples are intended to further illustrate the invention.

### EXAMPLES

### I. Materials and Methods

### 1. Organic Synthesis: Synthesis of SiFA-Br

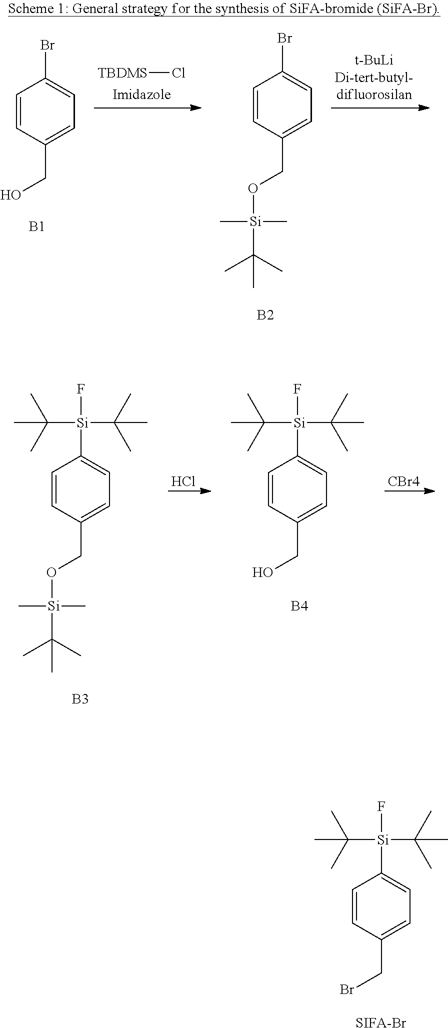

### ((4-Bromobenzyl)oxy)(tert-butyl)dimethylsilane (B2)

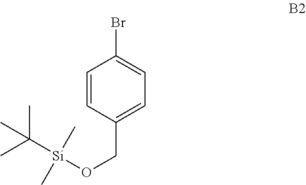 [SMILES: CC(C)(C)[Si](C)(C)OCc1ccc(Br)cc1]

In a round bottom flask, 4.68 g of 4-Bromobenzyl alcohol (B1, 25.0 mmol, 1.00 eq.) are dissolved and stirred in 70 mL dry DMF. 2.04 g of imidazole (30.0 mmol, 1.20 eq.) and 4.52 g TBDMS-chloride (30.0 mmol, 1.20 eq.) are added under stirring. The mixture is left for reaction for 20 h at room temperature. The reaction is then poured into 250 ml of ice-cold H₂O and the organic phase is extracted with Et₂O (5×50 mL). The combined organic phases are washed with a saturated aqueous solution of NaHCO₃ (100 mL), brine (100 mL) and dried over Na₂SO₄. The solvent is removed under reduced pressure and the crude product is purified via column chromatography (5% EtOAc in petroleum ether). After the solvents are removed under reduced pressure, 7.24 g of the product B2 (24.1 mmol, 96%) are yielded as a colorless oil.

TLC (SiO₂, 5% EtOAc/petroleum ether): R_{f}=0.97 [UV]

¹H-NMR (300 MHz, CDCl₃): δ[ppm]=7.45 (d, ³J=8 Hz, 2 H, H_{Ar}), 7.20 (d, ³J=8 Hz, 2 H, H_{Ar}), 4.68 (s, 2H, Ar—CH₂), 0.94 (s, 9H, C—CH₃), 0.10 (s, 6H, Si—CH₃).

¹³C-NMR (75 MHZ, CDCl₃): δ[ppm]=140.3 (s, Cᵢ), 130.1 (s, Cₘ), 127.5 (s, Cₒ) 120.4 (s, Cₚ), 64.2, (s, CH₂), 25.8 (s, C—CH₃), 18.2, (s, C—CH₃) 5.4 (s, Si—CH₃).

### Di-tert-butyl (4-(((tert-butyldimethylsilyl)oxy)methyl)phenyl) fluorosilane (B3)

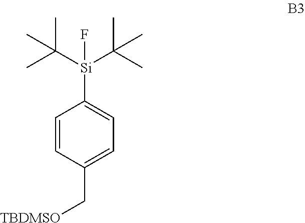 [SMILES: CC(C)(C)[Si](C)(C)OCc1ccc([Si](F)(C(C)(C)C)C(C)(C)C)cc1]

In an argon atmosphere, 7.24 g B2 (24.1 mmol, 1.00 eq.) are dissolved in 67 mL dry THF and cooled to −78° C. (dry ice and acetone). Over a period of 1.5 h, 32.6 mL of a 1.7 M solution of (BuLi in pentane (55.4 mmol, 2.30 eq.) are slowly dropped to the solution of B2 in THF. The mixture is left for stirring at −78° C. for an additional 30 min. In another round bottom flask, 5.00 g of di-tert-butyldifluorosilane (27.7 mmol, 1.10 eq.) are dissolved in 44 mL of dry THF and also cooled to −78° C. Over a period of 2 h and under constant stirring, the mixture of B2 and tBuLi in THF is slowly dropped to the solution of di-tert-butyldifluorosilane. The reaction is allowed to warm to room temperature and is left under stirring for another 15 h. Through addition of 120 mL of brine the reaction is terminated and the organic phase is separated. The aqueous phase is extracted with Et₂O (3× 100 mL), the combined organic phases are dried over MgSO₄ and the solvents are removed under reduced pressure. The product B3 is yielded as a yellowish oil (9.14 g, 23.9 mmol, 99%).

¹³C-NMR (75 MHZ, CDCl₃): δ[ppm]=143.0 (s, Cᵢ), 134.1 (d, ³ J(¹³C, ¹⁹F)=12 Hz, Cₘ), 132.0 (d, ²J(¹³C, ¹⁹F)=56 Hz, Cₚ), 125.3 (s, Cₒ), 65.0 (s, CH₂), 27.5 (s, CH₃), 26.8 (s, C—CH₃), 26.1 (s, CH₃), 20.4 (d, ²J(¹³C, ¹⁹F)=8 Hz, C—CH₃), 5.11 (s, Si—CH₃).

### (4-(Di-tert-butylfluorosilyl)phenyl) methanol (B4)

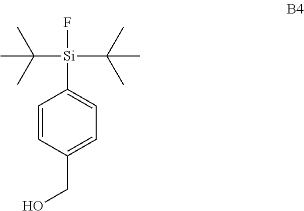 [SMILES: CC(C)(C)[Si](F)(c1ccc(CO)cc1)C(C)(C)C]

Compound B3 (9.14 g, 23.9 mmol, 1.00 eq.) is dissolved in 50 mL MeOH. After the addition of 3.00 mL of concentrated HCl (97.9 mmol, 4.10 eq.) the solution is left for reaction for 18 h at room temperature. The mixture is concentrated under reduced pressure, the precipitate is dissolved in 50 mL of Et₂O and the organic phase is washed with 50 ml of a saturated, aqueous solution of NaHCO₃. The aqueous phase is extracted with Et₂O (3×50 mL), the combined organic phases are combined and dried over MgSO₄. The solvents are removed under reduced pressure and the product B4 (5.90 g, 22.0 mmol, 92%) is yielded as a yellowish oil.

¹H-NMR (CDCl₃): δ[ppm]=7.61 (d, 2H, ³ J=8 Hz, H_{Ar}), 7.38 (d, 2H, ³J=8 Hz, H_{Ar}), 4.72 (s, 2H, Ar—CH₂), 1.06 (s, 18H, C—CH₃).

¹³C-NMR (CDCl₃): δ[ppm]=142.3 (s, Cᵢ), 134.4 (d, ³J(¹³C, ¹⁹F)=12 Hz, Cₘ), 133.1 (d, ²J (¹³C, ¹⁹F)=56 Hz, Cₚ), 125.6 (s, Cₒ), 65.4 (s, CH₂), 27.4 (s, CH₃), 20.4 (d, ²J(¹³C, ¹⁹F)=8 Hz, C—CH₃).

HPLC (50-100% B in 15 min) t_{R}=10.7 min

### (4-(Bromomethyl)phenyl)di-tert-butylfluorosilane (SiFA-Br)

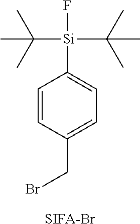

To a 0° C. cooled solution of B4 (3.08 g, 11.5 mmol, 1.0 eq.) and tetrabromomethane (4.18 g, 12.6 mmol, 1.1 eq.) in 100 mL DCM, triphenylphosphine (3.30 g, 12.6 mmol, 1.1 eq.) was added over a period of 30 min in small portions. The solution was stirred for 2 h at room temperature. Solvents were removed in vacuo and the residue was washed with cold n-hexane (3×50 mL). A white precipitate was removed by filtration and the solution was concentrated in vacuo. Purification was conducted by flash column chromatography (silica, 5% EtOAc in petrol, v/v). Compound SiFA-Br was isolated as a colorless oil (3.06 g, 9.20 mmol, 80%).

RP-HPLC (50 to 100% B in 15 min): t_{R}=9.2 min, K′=3.73.

¹H-NMR (400 MHZ, CDCl₃): δ[ppm]=7.58 (2 H, d, C6H₄), 7.40 (2 H, d, C6H₄), 4.49 (2 H, s, CH₂OSi), 1.05 (18 H, s, Si((Bu)₂).

### 2. General Methods

### 2.1 Solvents and Reagents

### Solvents

All solvents are used without further purification. They are purchased from Sigma-Aldrich Chemie GmbH (Munich, Germany) or VWR International GmbH (Bruchsal, Germany). Before usage, H₂O is purified by a Barnstead MicroPure-system from Thermo Fischer Scientific Inc.

(Waltham, USA). Final purification of products for quality control or complexation with ^{nat}Ga is executed in trace pure water from Merck Millipore (Darmstadt, Germany).

### Reagents for Peptide Synthesis

AAs are purchased from Iris Biotech GmbH (Marktredwitz, Germany), Sigma-Aldrich Chemie GmbH (Munich, Germany), or Merck Millipore (Darmstadt, Germany). Coupling reagents and chemicals are purchased from Sigma-Aldrich Chemie GmbH (Steinheim, Germany), Molekula GmbH (Garching, Germany), and Macrocyclics Inc. (Dallas, USA).

### Reagents for General Synthesis

Chemicals for general synthesis are purchased from Sigma-Aldrich Chemie GmbH (Steinheim, Germany) and Merck KGaA (Darmstadt, Germany). If not mentioned otherwise, the reagents are used without further purification.

### Chelators

The chelator DOTA((Bu)₂ is purchased from CheMatech (Dijon, France).

### Biochemicals

TABLE 1
Biochemicals and Contents.

| Product | Content [Vendor] |
|---|---|
| DMEM/F12 | Gibco Dulbecco's Modified Eagle Medium/Ham's F- |
| | 12 Nutrient Mixture (1:1) (1X) + GlutaMAX ™ [Gibco] |
| FCS | Fetal Calf Serum, superior [Gibco] |
| HBSS | Hanks' balanced salt solution, modified with |
| | NaHCO₃, without phenol red [Sigma Aldrich] |
| HBSA | HBSS with 1% w/v Bovine Serum Albumin (BSA) |
| | [Sigma Aldrich] |
| PBS | Dulbecco's Phosphate Buffered Saline w/o Ca²⁺ and |
| | Mg²⁺, endotoxin free [Sigma Aldrich] |
| RPMI | RPMI Medium 1640 (1x) + L-Glutamine [Gibco] |
| Trypan blue | Trypan blue solution 0.4% [Sigma Aldrich] |
| Trypsin/EDTA | 0.05% Trypsine, 0.02% Ethylenediaminetetraacetic |
| | acid (EDTA) in PBS w/o Ca²⁺ and Mg²⁺ [PAN |
| | Biotech] |

### 2.2 Instruments and Software

### High Performance Liquid Chromatography

High performance liquid chromatography (HPLC) is executed using analytical reversed-phase (RP)-HPLC with a linear gradient of MeCN (with 2% H₂O and 0.1% trifluoroacetic acid (TFA); v/v) in H₂O (with 0.1% TFA) in 15 min and a subsequent isocratic solvent mixture of 95% MeCN in H₂O (v/v) until completion (standard: 5 min). Detection occurs at λ=220 nm (peptide bonds) or λ=254 nm (aromatic systems). RP-HPLC chromatograms are analyzed using the LabSolution Software from Shimadzu Corp. (Kyoto, Japan). For analytical investigations two different systems are used:
1) Shimadzu Corp. (Kyoto, Japan): comprising of two LC-20AD gradient pumps, a CBM-20A communication module, a CTO-20A column oven, a SPD-20A ultraviolet/visible light (UV/VIS) detector, and a MultoKrom® 100-5 C18-column (125× 4.6 mm, 5 μm particle size, CS Chromatographie GmbH) with a flowrate of 1 ml/min.
2) Shimadzu Corp. (Kyoto, Japan): comprising of two LC-20AD gradient pumps, a CBM-20A communications module, a Smartline UV detector 2500 from the firm Dr. Ing. Herbert Knauer GmbH (Berlin, Germany) and a MultoKrom® 100-5 C18-column (125× 4.6 mm, 5 μm particle size, CS Chromatographie GmbH) with a flowrate of 1 ml/min.

The purification of final products is executed using preparative RP-HPLC with a linear gradient of MeCN (with 5% H₂O and 0.1% TFA; v/v) in H₂O (with 0.1% TFA; v/v) in 15 or 20 min and a subsequent isocratic solvent mixture of 95% MeCN in H₂O (v/v) until completion (standard: 5 min). Detection occurs at λ=220 nm (peptide bonds) or λ=254 nm (aromatic systems). RP-HPLC chromatograms are analyzed using the LabSolution Software from Shimadzu Corp. (Kyoto, Japan). For preparative purifications, three separate systems are used:
1) Shimadzu Corp. (Kyoto, Japan): comprising oft two LC-20AP gradient pumps, a DGU-20A degassing unit, a CBM-20A communication module, a CTO-20A column oven, an SPD-20A UV/VIS detector, and a multospher 100 C18-column (5 μm, 250×20 mm, CS Chromatography GmbH) with a flowrate of 8 ml/min.
2) Shimadzu Corp. (Kyoto, Japan): comprising of two LC-20AT gradient pumps, a DGU-20A degassing unit, a CBM-20A communication module, an SPD-20A UV/VIS detector, and a multospher 100 C18-column (5 μm, 250× 10 mm, CS Chromatographie GmbH) with a flowrate of 5 ml/min.
3) Shimadzu Corp. (Kyoto, Japan): comprising of two LC-20AP gradient pumps, a CBM-20A communication module, an SPD-20A UV/VIS detector, a SIL-10AP autosampler, an FRC-10A fraction collector, and a multospher 100 C18-column (5 μm, 250×20 mm, CS Chromatographie GmbH) with a flowrate of 8 ml/min.

The investigation of radioactive substances occurs using two different analytical radio RP-HPLC systems with a linear gradient of MeCN (with 2% H₂O and 0.1% TFA; v/v) in H₂O (with 0.1% TFA; v/v) in 15 min followed by an isocratic solvent mixture of 95% MeCN in H₂O (v/v) until completion (standard: 5 min). Detection occurs at λ=220 nm (peptide bonds) or λ=254 nm (aromatic systems) or using a radio-detector. The two systems are comprised of:
1) Shimadzu Corp. (Kyoto, Japan): comprising of two LC-20AD gradient pumps, a DGU-20A degassing unit, a SIL-20A autosampler, a CTO-10AS column oven, an FRC-10A fraction collector, an SPD-20A UV/VIS detector, a HERM LB500 (Nal-scintillation crystal) radio-detector from the firm Berthold Technologies GmbH (Bad Wilbad, Germany) a CBM-20A communications module, and a Multospher® 100 RP18 column (5 μm, 125×4.6 mm, CS Chromatographie GmbH).
2) Shimadzu Corp. (Kyoto, Japan): comprising of two LC-20AD gradient pumps, an SPD-20A UV/VIS detector, a HERM LB500 (Nal-scintillation crystal) radio-detector from the firm Berthold Technologies GmbH (Bad Wilbad, Germany) a CBM-20A communications module, and a MultoKrom® 100-5 C18-column (125× 4.6 mm, 5 μm particle size, CS Chromatographie GmbH).

The capacity factors (K′) are calculated as follows:

$${K}^{′} = \frac{{t}_{R} [ min ] - {t}_{0} [ min ]}{{t}_{0} [ min ]}$$

with the experimentally determined retention time (t_{R}) and the experimentally determined dead time (t₀) of the respective column.

### Determination of the HSA Binding

For the determination of the percentual HSA binding, the Shimadzu analytical chromatography system 1 is used in combination with a chiral HSA-column (5 μm, 50×3 mm) from Chiral Technologies Europa SAS (Illkirchen-Graffenstaden, France) with a flowrate of 0.5 ml/min. The solvents are exchanged for an aquatic 50 mM NH₄OAc-solution (pH=6.9) and iPrOH. Gradient: 0-3 min: 0-100% NH₄OAc in iPrOH; then isocratically 80% NH₄OAc in iPrOH.

### Electrospray Ionization-Mass Spectrometry

Mass spectrometry (MS) is executed using a Varian 500-MS IT mass spectrometer with electrospray ionization (ESI) and ion trap-detector from Agilent Technologies (Santa Clara, USA).

### Radio-RP-Thin-Layer Chromatography

Radio-RP thin-layer chromatography (TLC) is executed on Silica gel 60 RP-18 F_{254S} TLC strips (1×10 cm) from Merck Millipore (Darmstadt, Germany) and analyzed using a Scan-RAM Radio TLC-Detector and the Laura software from LabLogic Systems Ltd (Sheffield, UK).

### γ-Counter

To quantify radioactive samples, a model 2480 Wizard2 γ-counter from the company PerkinElmer Inc. (Waltham, USA) is used.

### Dose Calibrator

To estimate the activity of radioactive samples, a CRC®-55tW Dose Calibrator/Well counter from Mirion Technologies (Florham Park, USA) is used.

### Incubator

Cultivation and incubation of Chinese hamster ovary (CHO) and AR42J-cells occurs in a HERAcell 150i-incubator from Thermo Fischer Scientific Inc. (Waltham, USA) at 37° C. and in an atmosphere containing 5% CO₂.

### Lyophilizer

Freeze-drying of intermediate and final products is executed at an Alpha 1-2 lyophilizer from the company Christ (Osterode am Harz, Germany) coupled to an Edwards nXDS10i vacuum pump from Edwards Limited (Burgess Hill, UK).

### 1.3 Radioactive Nuclides

### ¹⁸F

Radioactive [¹⁸F] F⁻ was purchased from the clinic Rechts der Isar (Munich, Germany) and delivered in a 2.5 ml aqueous solution (˜4-10 GBq).

### ¹²⁵I

Radioactive iodinations with ¹²⁵I were executed with a [¹²⁵I] Nal-solution in 40 mM NaOH (74 TBq/mmol) from the company HARTMANN ANALYTIC GmbH (Braunschweig, Germany).

### 3 General Synthetic Procedures (GSP)

### GSP1 Resin Loading of the 2-CTC Resin

The 2-Chlorotrityl chloride resin (2-CTC; maximal occupancy: 1.6 mmol/g) (1 equivalent (eq.), 1 g) is given to a solution of N,N-Diisopropylethylamine (DIPEA, 2.25 eq.) and the Fmoc-protected AA in N,N-dimethylformamide (DMF, total volume: ˜ 15 ml) and stirred at room temperature (RT) for 3 h. MeOH (4 ml) is added to the solution and stirred for 15 min at RT. The resin is washed in solutions with an increasing percentage of MeOH in DMF (25%, 50%, 75%, 100%) and dichloromethane (DCM, 5×15 ml). The resin is dried in a desiccator overnight.

The resin occupancy is determined according to the following equation:

$$l [ \frac{mmol}{g} ] = \frac{( {m}_{1} - {m}_{2} ) \cdot 1000}{( M - {M}_{HCl} ) \cdot {m}_{2}}$$

P With the occupancy

$$l [ \frac{mmol}{g} ] ,$$

the resin-mass ere van m₁[g], the resin-mass after loading m₂ [g], the molar mass of the loading-molecule

$$M [ \frac{g}{mol} ]$$

and the molar mass of HCl

$${M}_{HCl} = 36.4 \; 61 \frac{g}{mol} .$$

### GSP2 Fmoc Deprotection

N-Terminal deprotection of Fmoc protected amines occurs by the addition of 10 ml of piperidine (20% in DMF; v/v). Addition of the solution occurs twice (1×15 min, 1×5 min) with subsequent washing of the resin (6×5 ml DMF, 4×5 ml DCM). The resin is then either used in a following reaction or dried in a desiccator overnight.

To prevent elimination reactions in SiFAlin-containing molecules, the final deprotection of Fmoc protected amines occurs for no longer than 5 min with the previously described method using piperidine (20% in DMF; v/v).

### GSP3 Dde Deprotection

For the deprotection of the Dde- group, a solution of hydroxylamine hydrochloride (1.25 g) and imidazole (0.92 g) in N-methyl-2-pyrrolidine (NMP; 5 ml) and DCM (1 ml) is prepared.

The resin was swollen in DMF and shaken in the mixture for 3 h. The resin was washed with NMP(4×5 ml), DMF (4×5 ml), DCM (4×5 ml) and dried in a desiccator overnight.

### GSP4 Standard Solid-Phase Peptide Coupling

The loaded resin is swollen in DMF for 30 min. A solution of the Fmoc-protected AA (1.5 eq.), O-(Benzotriazol-1-yl)-N,N,N′,N′-tetramethyluroniumtetrafluoroborate (TBTU; 1.5 eq.),
1-hydroxy-7-azabenzotriazole (HOAt, 1.5 eq.) and DIPEA (4.5 eq.) in DMF is pre-activated (10 min) and added to the resin. The solution is shaken for 2 h and the resin washed with DMF (6×5 ml) and DCM (4×5 ml). The resin-containing syringes are dried in a desiccator overnight.

### GSP5 Coupling of Fmoc-L-Cys (Acm)-OH AAs

The loaded resin is swollen in DMF for 30 min. A solution of Fmoc-L-Cys (Acm)-OH (2.0 eq.), N,N′-diisopropylcarbodiimide (DIC, 4.0 eq.), ethyl cyanohydroxyiminoacetate (Oxyma) (2.0 eq.) and DIPEA (0.8 eq.) in DMF is pre-activated (2 min) and added to the resin. The solution is shaken for 2 h and the resin washed with DMF (6×5 ml) and DCM (4×5 ml). The resin-containing syringes are dried in a desiccator overnight.

### GSP6 Coupling of Dap AAs

The loaded resin is swollen in DMF for 30 min. A solution of the Fmoc-protected Dap AA (1.5 eq.), TBTU (1.5 eq.), HOAt (1.5 eq.), and sym-collidine (5.0 eq.) in DMF is pre-activated (2 min) and added to the resin. The solution is shaken for 2 h and the resin washed with DMF (6×5 ml) and DCM (4×5 ml). The resin-containing syringes are dried in a desiccator overnight.

### GSP7 Coupling of DOTA(tBu)₂

For the coupling of trans-(Di-tert-butyl)-1,4,7,10-tetraazacycIododecane-1,4,7,10-tetraacetic acid (DOTA(tBu)₂), a solution of DOTA((Bu)₂ (3.0 eq.), HOAt (3.0 eq.) and TBTU (3.0 eq.) with sym-collidine (11.0 eq.) in DMF is prepared and preactivated for 10 min. The solution is added to the Fmoc-deprotected, swollen resin, and stirred overnight. The resin is washed with DMF (6×5 ml) and DCM (4×5 ml). The resin-containing syringes are dried in a desiccator.

### GSP8 Coupling of SiFA-Br

For the coupling with (4-(bromomethyl)phenyl)di-tert-butylfluorosilane (SiFA-Br), the resin was swollen in DCM. A solution of DIPEA (6 eq.) and SiFA-Br (3 eq.) were added to the solution in DCM (2 ml) and stirred overnight. The resin was washed with DCM (5×5 ml) and dried in a desiccator.

GSP9 Cyclization with Thallium (III) Trifluoroacetate

The resin is swollen in DMF for 30 min. A solution of Thallium (III) trifluoroacetate (TTFA) (2 eq.) and glycerol (4 eq.) in DMF (8 ml+2 ml) is prepared and given to the swollen resin. The suspension is stirred for 1 h. Then, the solution is exchanged for a fresh solution and stirred for 1 h. The resin is washed with DMF (10×8 ml) and DCM (5×8 ml) and dried in a desiccator overnight.

### GSP10 Resin Cleavage Under Retention of Acid Labile Protecting Groups

To the dried resin, 10 ml of a solution of hexafluoroisopropanol (HFIP; 20% in DCM; v/V) is given and shaken for 45 min. The procedure is repeated, and the resin washed with DCM (3×5 ml). The combined solutions are collected in a round bottom flask and the volatile components evaporated under reduced pressure.

GSP11 Resin Cleavage under Cleavage of Acid Labile Protecting Groups

A solution of TFA (87.5%), triisopropylsilane (TIPS; 2.5%), and H₂O (10%) is given to the resin. After incubation (2×45 min), the resin is washed with TFA (5 ml), and all fractions collected in a round bottom flask. The volatile components are evaporated in a N2-stream, giving the crude product.

GSP12 Complexation with ^{nat}Ga

For the incorporation of ^{nat}Ga into the chelator, a 2 mm solution of the compound in dimethylsulfoxide (DMSO) is combined with a solution of Ga(NO₃) 3 (20 mm in H₂O, 1.5 eq.) and dissolved to 1 mM by the addition of DMSO. The mixture is incubated at 70° C. for 1 h yielding the product.

### GSP13 Freeze-Drying

The dried product is dissolved in a small amount of fBuOH and H₂O and frozen at −80° C. The volatile components are completely removed under vacuum (lyophilized).

GSP14 Complexation with ^{nat}Lu

For the incorporation of ^{nat}Lu into the chelator, a 2 mm solution of the compound in dimethylsulfoxide (DMSO) is combined with a solution of LuCl₃ (20 mm in H₂O, 1.5 eq.) and dissolved to 1 mM by the addition of DMSO. The mixture is incubated at 90° C. for 1 h yielding the product.

### 4. Synthesis of Fmoc-TATE (PG)-2-CT

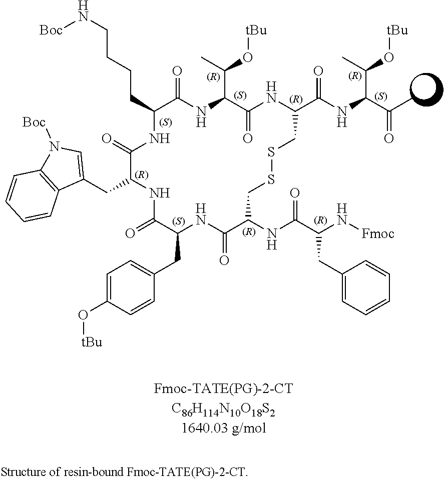

The synthesis of resin-bound Fmoc-TATE (PG)-2-CT is executed according to a procedure by Niedermoser et al [23]. 2-CTC resin is loaded with Fmoc-L-Thr((Bu)-OH according to GSP1

(resin occupancy: 0.5-0.7 mmol/g). After Fmoc-deprotection (GSP2), Fmoc-L-Cys (Acm)-OH (GSP5) is coupled followed by Fmoc-L-Thr(tBu)-OH (GSP2, GSP4), Fmoc-L-Lys(Boc)-OH (GSP2, GSP4), Fmoc-D-Trp (Boc)-OH (GSP2, GSP4), Fmoc-L-Tyr((Bu)-OH (GSP2, GSP4), Fmoc-L-Cys (Acm)-OH (GSP2, GSP5) and Fmoc-D-Phe-OH (GSP2, GSP4). Oxidative cyclization of the resulting peptide-chain with simultaneous deprotection of the Acm-protecting groups occurs according to GSP9, yielding the resin-bound Fmoc-TATE (PG)-2-CT. Test-cleavage occurs under acidic conditions with TFA (10 min, RT). The formation of the correct product is confirmed using analytical RP-HPLC and ESI-MS.

Fmoc-D-Phe-cyclo[L-Cys-L-Tyr((Bu)-D-Trp (Boc)-L-Lys(Boc)-L-Thr((Bu)-L-Cys]-L-Thr-OH

RP-HPLC (analytical): (10-90% MeCN/H₂O with 0.1% TFA, v/v, 15 min): t_{R}=10.8 min; K′=4.1.

MS (ESI positive): m/z calculated for C₆₄H₇₄N₁₀O₁₄S₂: 1270.48, found: 1315.1 [M+CO₂+H]⁺.

### 5. Synthesis of Ligands

### 5.1 Synthesis of 01

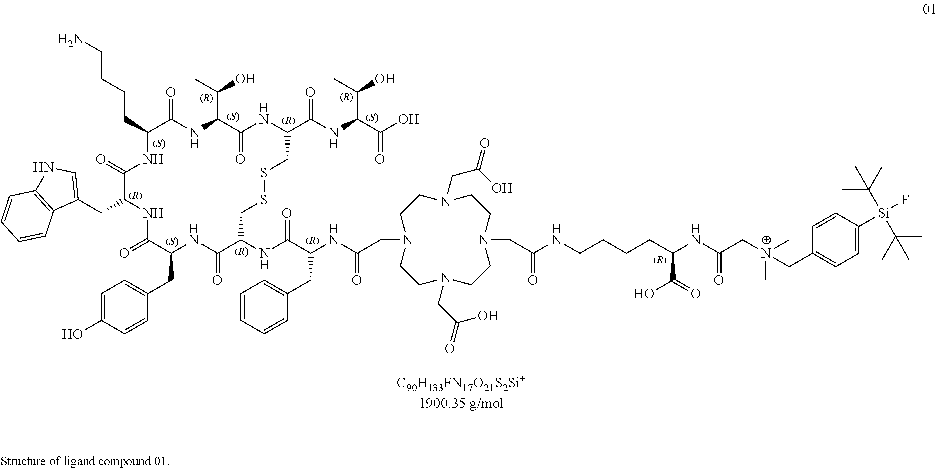

01 is synthesized starting from the 2-CT-TATE (PG)-Fmoc precursor described in Chapter4. The precursor is Fmoc-deprotected (GSP2) and coupled to DOTA((Bu)₂ (GSP7). A solution of TBTU (1.5 eq.), HOAt (1.5 eq.), and DIPEA (4.5 eq.) in DMF (2.5 ml) is added to the resin and preactivated for 10 min. A solution of Fmoc-D-Lys-OfBu (1.5 eq) in DMF (2.5 ml) is added to the preactivated resin and stirred for 2 h. The resin is washed with DMF (5×10 ml). Dimethylglycine hydrochloride (GSP2, GSP4) is coupled followed by SiFA-Br (GSP8). The product is cleaved from the resin with simultaneous deprotection of all acid-labile groups (GSP11), purified via preparative RP-HPLC, and freeze-dried (GSP13). The formation of the correct product is confirmed by QC using analytical RP-HPLC and ESI-MS.

01 (N-SiFAlin-N,N-Me2-Gly-D-Lys(trans-DOTA-TATE)-OH):

RP-HPLC (analytical): (10-60% MeCN/H₂O with 0.1% TFA, v/v, 15 min): t_{R}=12.7 min; K′=5.0.

RP-HPLC (preparative): (33-50% MeCN/H₂O with 0.1% TFA, v/v, 20 min): t_{R}=18.5 min; K′=5.5.

MS (ESI positive): m/z calculated for C₉₀H₁₃₃FN₁₇O₂₁S₂Sit: 1898.91, found: 634.0 [M+3H]³⁺, 950.3 [M+2H]²⁺, 1899.8 [M+H]⁺.

### 5.2 Synthesis of 02

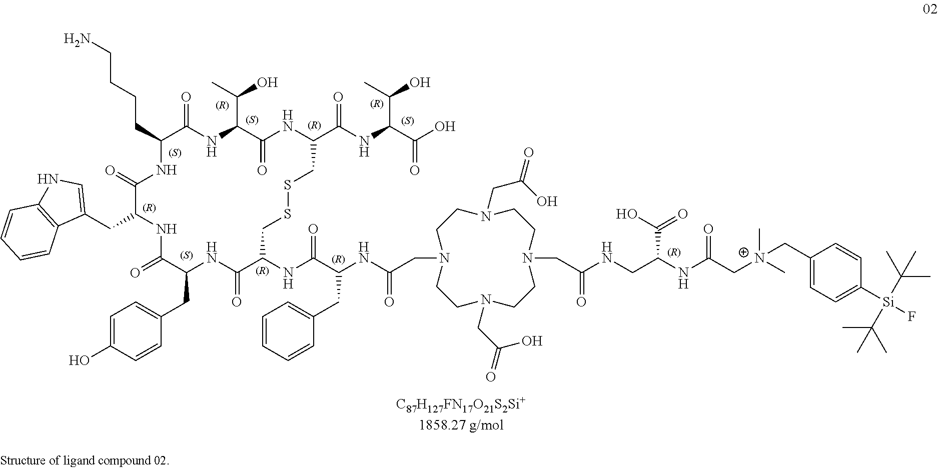

Ligand 02 is synthesized starting from the Fmoc-TATE (PG)-2-CT precursor described in Chapter 4. The precursor is Fmoc-deprotected (GSP2) and coupled to DOTA((Bu)₂ (GSP7). A solution of TBTU (1.5 eq.), HOAt (1.5 eq.), and DIPEA (4.5 eq.) in DMF (2.5 ml) is added to the resin and preactivated for 10 min. A solution of Fmoc-D-Dap-OfBu (1.5 eq) in DMF (2.5 ml) is added to the preactivated resin and stirred for 2 h. The resin is washed with DMF (5× 10 ml). Dimethylglycine hydrochloride (GSP2, GSP4) is coupled followed by SiFA-Br (GSP8). The product is cleaved from the resin with simultaneous deprotection of all acid-labile groups (GSP11), purified via preparative RP-HPLC, and freeze-dried (GSP13). The formation of the correct product is confirmed by quality control (QC) using analytical RP-HPLC and ESI-MS.

02 (N-SiFAlin-N,N-Me2-Gly-D-Dap(trans-DOTA-TATE)-OH):

RP-HPLC (analytical): (10-60% MeCN/H₂O with 0.1% TFA, v/v, 15 min): t_{R}=12.6 min; K′=5.0.

RP-HPLC (preparative): (35-47% MeCN/H₂O with 0.1% TFA, v/v, 20 min): t_{R}=17.2 min; K′=5.2.

MS (ESI positive): m/z calculated for C₈₇H₁₂₇FN₁₇O21S₂Si⁺: 1856.86, found: 619.9 [M+3H]³⁺, 929.3 [M+2H]²⁺.

### 5.3 Synthesis of 03

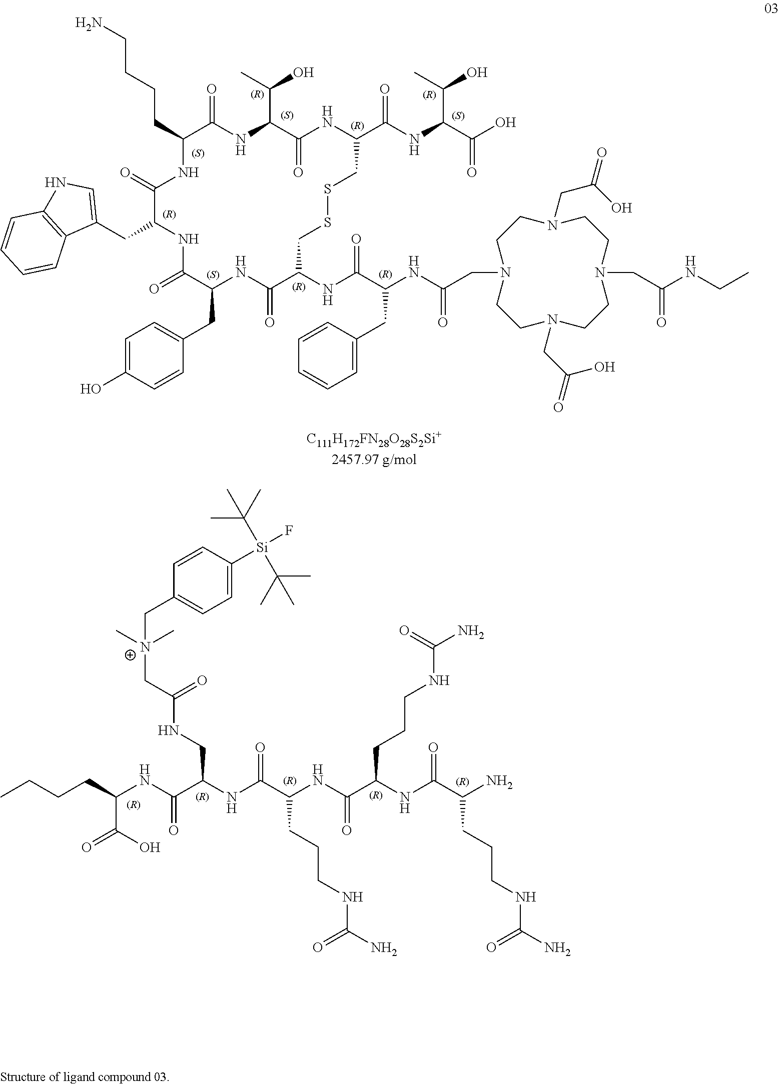

03 is synthesized starting from Fmoc-TATE (PG)-2-CT precursor described in Chapter 4. The precursor is Fmoc-deprotected (GSP2) and coupled to DOTA((Bu)₂ (GSP7). A solution of TBTU (1.5 eq.), HOAt (1.5 eq.), and DIPEA (4.5 eq.) in DMF (2.5 ml) is added to the resin and preactivated for 10 min. A solution of Fmoc-D-Lys-OtBu (1.5 eq.) in DMF (2.5 ml) is added to the preactivated resin and stirred for 2 h. The resin is washed with DMF (6×5 ml) and Fmoc-D-Dap(Dde)-OH is coupled (GSP6). The Dde- group is cleaved (GSP3) and dimethylglycine hydrochloride is coupled (GSP4). After Fmoc-deprotection (GSP2), Fmoc-D-Cit-OH is coupled (GSP4), followed by Fmoc-D -Cit-OH (GSP2, GSP4) and Fmoc-D -Cit-OH (GSP2, GSP4). SiFA-Br is coupled (GSP8) and the final Fmoc- group removed (GSP2). The product is cleaved from the resin with simultaneous deprotection of all acid-labile groups

(GSP11), purified via preparative RP-HPLC, and freeze-dried (GSP13). The formation of the correct product is confirmed by QC using analytical RP-HPLC and ESI-MS.

03 (H-D -Cit-D -Cit-D -Cit-D-Dap(N-SiFAlin-N,N-Me2-Gly)-D-Lys(trans-DOTA-TATE)-OH):

RP-HPLC (analytical): (10-60% MeCN/H₂O with 0.1% TFA, v/v, 15 min): t_{R}=11.2 min; K′=4.3.

RP-HPLC (preparative): (33-41% MeCN/H₂O with 0.1% TFA, v/v, 20 min): t_{R}=15.9 min; K′=5.1.

MS (ESI positive): m/z calculated for C₁₁₁H₁₇₂FN₂₈O28S₂Si⁺: 2456.21, found: 819.9 [M+3H]³⁺, 1229.1 [M+2H]²⁺, 1639.2 [2M+3H]³⁺, 1843.9 [3M+4H]⁴⁺

### 5.4 Synthesis of 04

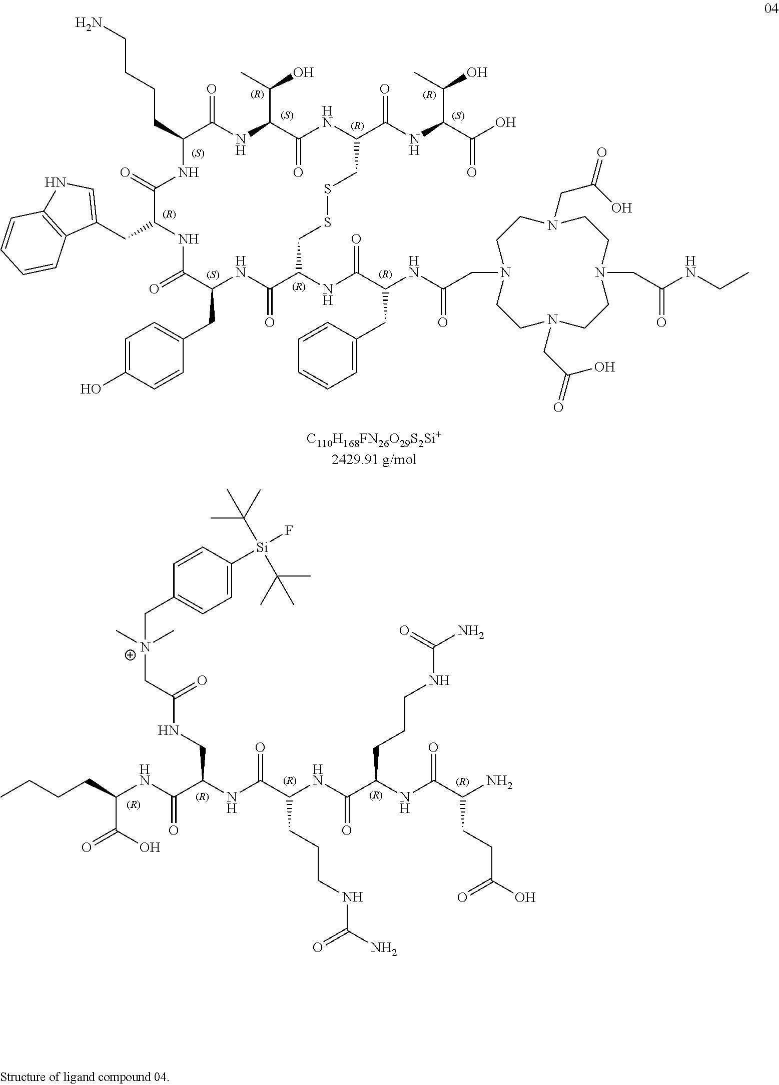

04 is synthesized starting from Fmoc-TATE (PG)-2-CT precursor described in Chapter 4. The precursor is Fmoc-deprotected (GSP2) and coupled to DOTA((Bu)₂ (GSP7). A solution of TBTU (1.5 eq.), HOAt (1.5 eq.), and DIPEA (4.5 eq.) in DMF (2.5 ml) is added to the resin and preactivated for 10 min. A solution of Fmoc-D-Lys-OtBu (1.5 eq.) in DMF (2.5 ml) is added to the preactivated resin and stirred for 2 h. The resin is washed with DMF (6×5 ml) and Fmoc-D-Dap(Dde)-OH is coupled (GSP6). The Dde- group is cleaved (GSP3) and dimethylglycine hydrochloride is coupled (GSP4). After Fmoc-deprotection (GSP2), Fmoc-D-Cit-OH is coupled (GSP4), followed by Fmoc-D -Cit-OH (GSP2, GSP4) and Fmoc-D-Glu((Bu)-OH (GSP2, GSP4). SiFA-Br is coupled (GSP8) and the final Fmoc- group removed (GSP2). The product is cleaved from the resin with simultaneous deprotection of all acid-labile groups (GSP11), purified via preparative RP-HPLC, and freeze-dried (GSP13). The formation of the correct product is confirmed by QC using analytical RP-HPLC and ESI-MS.

04 (H-D-Glu-D -Cit-D -Cit-D-Dap(N-SiFAlin-N,N-Me2-Gly)-D-Lys(trans-DOTA-TATE)-OH):

RP-HPLC (analytical): (10-60% MeCN/H₂O with 0.1% TFA, v/v, 15 min): t_{R}=11.3 min; K′=4.4.

RP-HPLC (preparative): (33-40% MeCN/H₂O with 0.1% TFA, v/v, 20 min): t_{R}=17.1 min; K′=5.4.

MS (ESI positive): m/z calculated for C₁₁₀H₁₆₈FN₂₆O29S₂Si⁺: 2428.17, found: 810.3 [M+3H]³⁺, 1215.2 [M+2H]²⁺, 1619.9 [2M+3H]³⁺.

### 5.5 Synthesis of 05

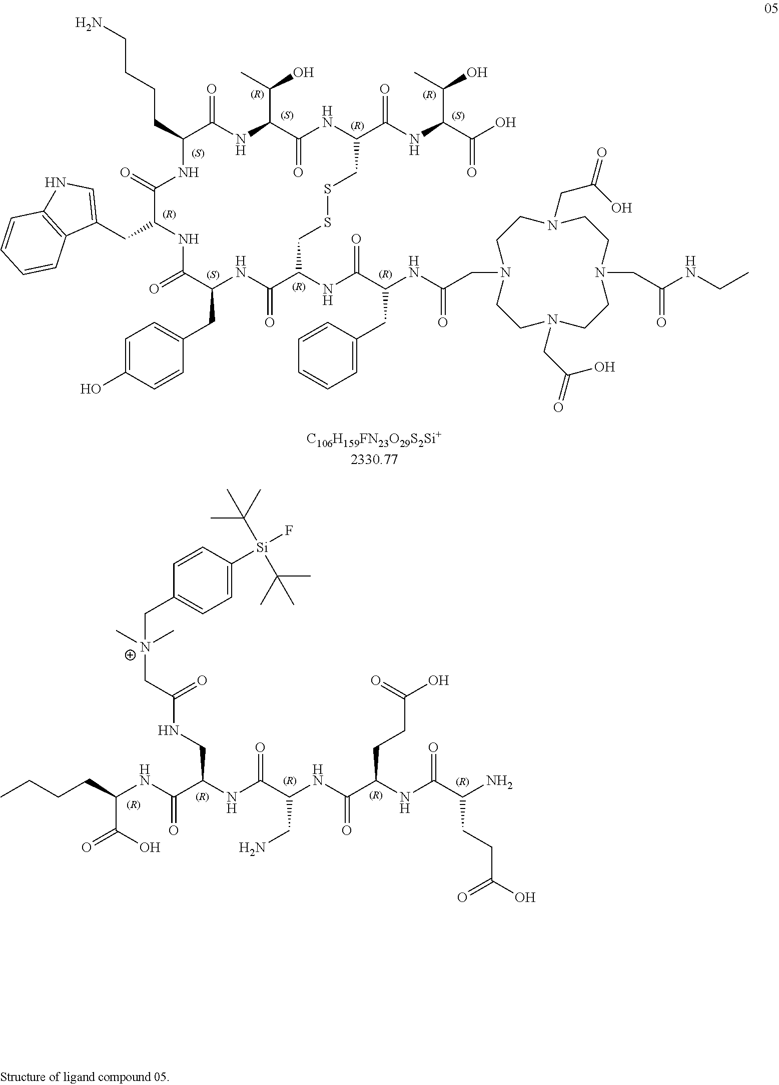

05 is synthesized starting from Fmoc-TATE (PG)-2-CT precursor described in Chapter 4. The precursor is Fmoc-deprotected (GSP2) and coupled to DOTA((Bu)₂ (GSP7). A solution of TBTU (1.5 eq.), HOAt (1.5 eq.), and DIPEA (4.5 eq.) in DMF (2.5 ml) is added to the resin and preactivated for 10 min. A solution of Fmoc-D-Lys-OfBu (1.5 eq.) in DMF (2.5 ml) is added to the preactivated resin and stirred for 2 h. The resin is washed with DMF (6×5 ml) and Fmoc-D-Dap(Dde)-OH is coupled (GSP6). The Dde- group is cleaved (GSP3) and dimethylglycine hydrochloride is coupled (GSP4). After Fmoc-deprotection (GSP2), Fmoc-D-Dap(Boc)-OH is coupled (GSP6), followed by Fmoc-D-Glu(fBu)-OH (GSP2, GSP4) and Fmoc-D-Glu((Bu)-OH (GSP2, GSP4). SiFA-Br is coupled (GSP8) and the final Fmoc- group removed (GSP2). The product is cleaved from the resin with simultaneous deprotection of all acid-labile groups (GSP11), purified via preparative RP-HPLC, and freeze-dried (GSP13). The formation of the correct product is confirmed by QC using analytical RP-HPLC and ESI-MS.

05 (H-D-Glu-D-Glu-D-Dap-D-Dap(N-SiFAlin-N,N-Mez-Gly)-D-Lys(trans-DOTA-TATE)-OH):

RP-HPLC (analytical): (10-60% MeCN/H₂O with 0.1% TFA, v/v, 15 min): t_{R}=11.2 min; K′=4.3.

RP-HPLC (preparative): (30-43% MeCN/H₂O with 0.1% TFA, v/v, 20 min): t_{R}=17.6 min; K′=5.8.

MS (ESI positive): m/z calculated for C₁₀₆H₁₅₉FN₂₃O₂₉S₂Si⁺: 2329.09, found: 583.2 [M+4H]⁴⁺, 77.1 [M+3H]³⁺, 1165.2 [M+2H]²⁺, 1553.6 [2M+3H]³⁺, 1748.9 [3M+4H]⁴⁺.

### 5.6 Synthesis of 06

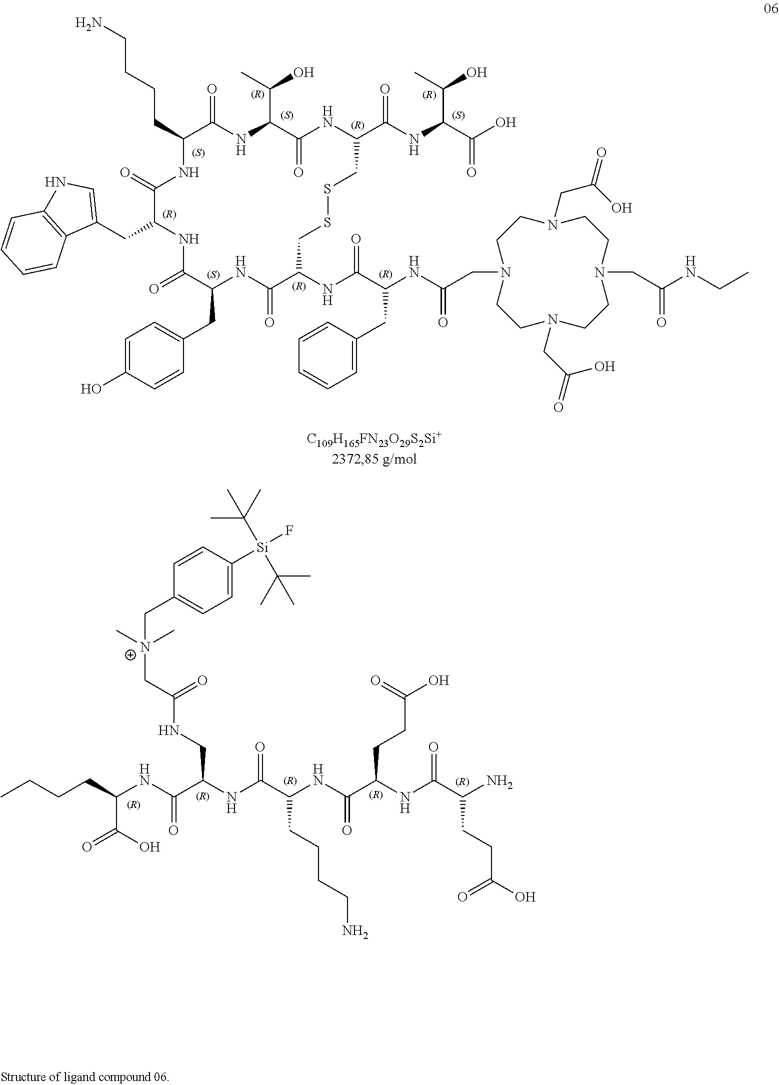

06 is synthesized starting from Fmoc-TATE (PG)-2-CT precursor described in Chapter 4. The precursor is Fmoc-deprotected (GSP2) and coupled to DOTA((Bu)₂ (GSP7). A solution of TBTU (1.5 eq.), HOAt (1.5 eq.), and DIPEA (4.5 eq.) in DMF (2.5 ml) is added to the resin and preactivated for 10 min. A solution of Fmoc-D-Lys-OfBu (1.5 eq.) in DMF (2.5 ml) is added to the preactivated resin and stirred for 2 h. The resin is washed with DMF (6×5 ml) and Fmoc-D-Dap(Dde)-OH is coupled (GSP6). The Dde- group is cleaved (GSP3) and dimethylglycine hydrochloride is coupled (GSP4). After Fmoc-deprotection (GSP2), Fmoc-D-Lys(Boc)-OH is coupled (GSP4), followed by Fmoc-D-Glu((Bu)-OH (GSP2, GSP4) and Fmoc-D-Glu((Bu)-OH (GSP2, GSP4). SiFA-Br is coupled (GSP8) and the final Fmoc- group removed (GSP2). The product is cleaved from the resin with simultaneous deprotection of all acid-labile groups (GSP11), purified via preparative RP-HPLC, and freeze-dried (GSP13). The formation of the correct product is confirmed by QC using analytical RP-HPLC and ESI-MS.

06 (H-D-Glu-D-Glu-D-Lys-D-Dap(N-SiFAlin-N,N-Mez-Gly)-D-Lys(trans-DOTA-TATE)-OH):

RP-HPLC (analytical): (10-60% MeCN/H₂O with 0.1% TFA, v/v, 15 min): t_{R}=11.1 min; K′=4.3.

RP-HPLC (preparative): (30-47% MeCN/H₂O with 0.1% TFA, v/v, 20 min): t_{R}=15.7 min; K′=4.7.

MS (ESI positive): m/z calculated for C₁₀₉H₁₆₅FN₂₃O₂₉S₂Si⁺: 2371.13, found: 791.2 [M+3H]³⁺, 1186.8 [M+2H]²⁺, 1582.5 [2M+3H]³⁺.

### 5.7 Synthesis of 07

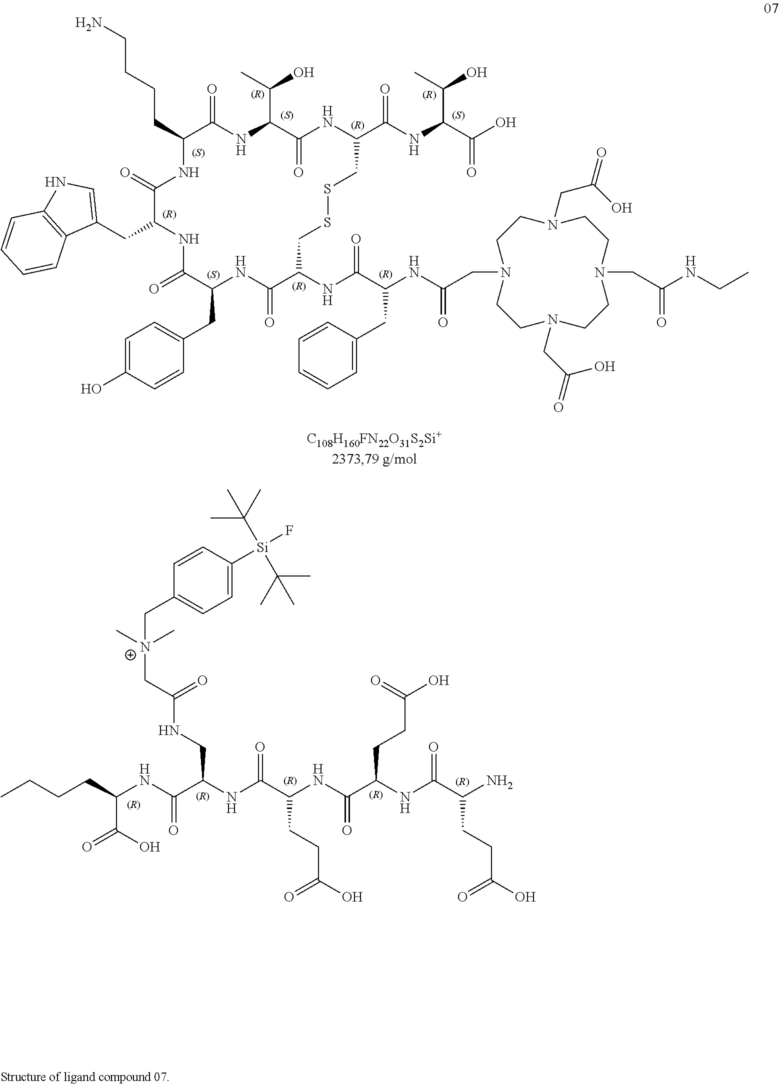

07 is synthesized starting from Fmoc-TATE (PG)-2-CT precursor described in Chapter 4. The precursor is Fmoc-deprotected (GSP2) and coupled to DOTA(Bu)₂ (GSP7). A solution of TBTU (1.5 eq.), HOAt (1.5 eq.), and DIPEA (4.5 eq.) in DMF (2.5 ml) is added to the resin and preactivated for 10 min. A solution of Fmoc-D-Lys-OfBu (1.5 eq.) in DMF (2.5 ml) is added to the preactivated resin and stirred for 2 h. The resin is washed with DMF (6×5 ml) and Fmoc-D-Dap(Dde)-OH is coupled (GSP6). The Dde- group is cleaved (GSP3) and dimethylglycine hydrochloride is coupled (GSP4). After Fmoc-deprotection (GSP2), Fmoc-D-Glu((Bu)-OH is coupled (GSP4), followed by Fmoc-D-Glu((Bu)-OH (GSP2, GSP4) and Fmoc-D-Glu((Bu)-OH (GSP2, GSP4). SiFA-Br is coupled (GSP8) and the final Fmoc- group removed (GSP2). The product is cleaved from the resin with simultaneous deprotection of all acid-labile groups (GSP11), purified via preparative RP-HPLC, and freeze-dried (GSP13). The formation of the correct product is confirmed by QC using analytical RP-HPLC and ESI-MS.

07 (H-D-Glu-D-Glu-D-Glu-D-Dap(N-SiFAlin-N,N-Me2-Gly)-D-Lys(trans-DOTA-TATE)-OH):

RP-HPLC (analytical): (10-60% MeCN/H₂O with 0.1% TFA, v/v, 15 min): t_{R}=11.5 min; K′=4.5.

RP-HPLC (preparative): (30-43% MeCN/H₂O with 0.1% TFA, v/v, 20 min): t_{R}=17.2 min; K′=5.3.

MS (ESI positive): m/z calculated for C₁₀₈H₁₆₀FN₂₂O₃₁S₂Si⁺: 2372.08, found: 593.2 [M+4H]⁴⁺, 791.0 [M+3H]³⁺, 1185.9 [M+2H]²⁺, 1581.1 [2M+3H]³⁺, 1779.2 [3M+4H]⁴⁺

### 5.8 Synthesis of 08

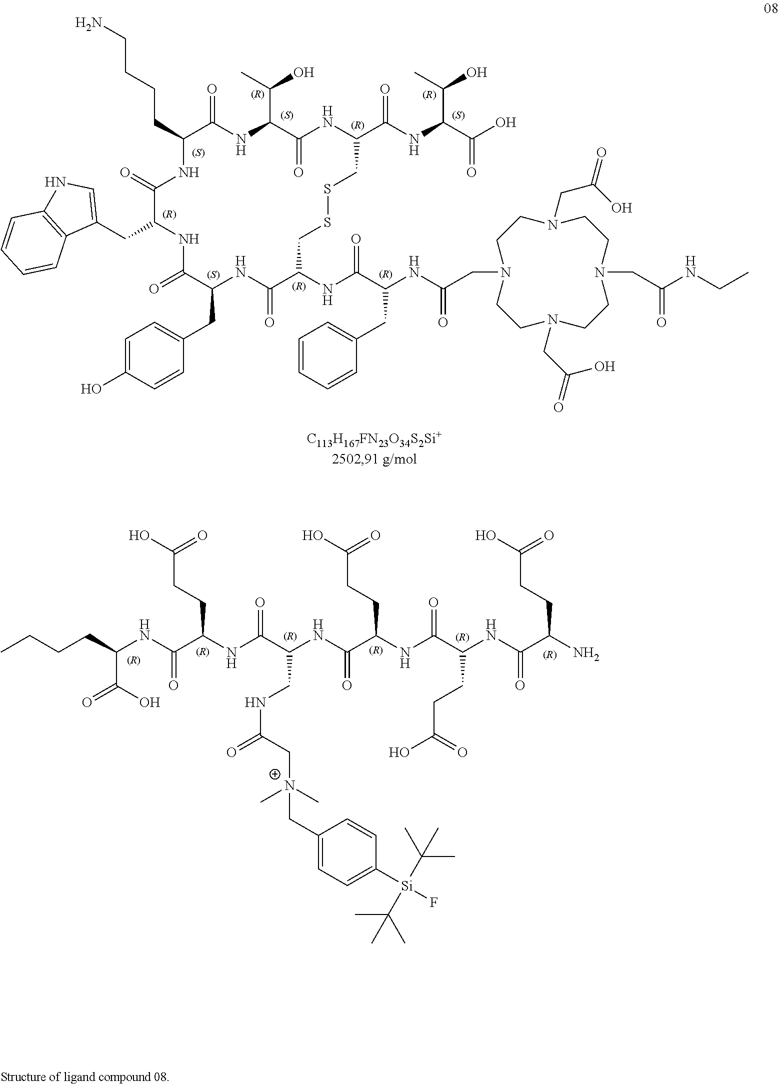

08 is synthesized starting from Fmoc-TATE (PG)-2-CT precursor described in Chapter 4. The precursor is Fmoc-deprotected (GSP2) and coupled to DOTA((Bu)₂ (GSP7). A solution of TBTU (1.5 eq.), HOAt (1.5 eq.), and DIPEA (4.5 eq.) in DMF (2.5 ml) is added to the resin and preactivated for 10 min. A solution of Fmoc-D-Lys-OfBu (1.5 eq.) in DMF (2.5 ml) is added to the preactivated resin and stirred for 2 h. After washing with DMF (6×5 ml) the compound is coupled to Fmoc-D-Glu((Bu)-OH (GSP2, GSP4), and Fmoc-D-Dap(Dde)-OH (GSP2, GSP6). The Dde- group is cleaved (GSP3) and dimethylglycine hydrochloride is coupled (GSP4). After Fmoc-deprotection (GSP2), Fmoc-D-Glu((Bu)-OH is coupled three times (GSP4) with intermittent Fmoc-deprotection (GSP2). After the coupling of SiFA-Br (GSP8), the final Fmoc- group is removed (GSP2). The product is cleaved from the resin with simultaneous cleavage of the acid-labile protecting groups (GSP11), purified via RP-HPLC, and lyophilized. The formation of the correct product is confirmed by QC using analytical RP-HPLC and ESI-MS.

08 (H-D-Glu-D-Glu-D-Glu-D-Dap(N-SiFAlin-N,N-Me2-Gly)-D-Glu-D-Lys(trans-DOTA-TATE)-OH: RP-HPLC (analytical): (10-60% MeCN/H₂O with 0.1% TFA, v/v, 15 min): t_{R}=11.2 min; K′=4.3.

RP-HPLC (preparative): (30-60% MeCN/H₂O with 0.1% TFA, v/v, 20 min): t_{R}=18.1 min; K′=2.1.

MS (ESI positive): m/z calculated for C₁₁₃H₁₆₇FN₂₃O₃₄S₂Si⁺: 2501.12, found: 625.8 [M+4H]⁴⁺, 834.0 [M+3H]³⁺, 1250.5 [M+2H]²⁺, 1667.1 [2M+3H]³⁺, 1875.3 [3M+4H]⁴⁺.

### 5.9 Synthesis of 09

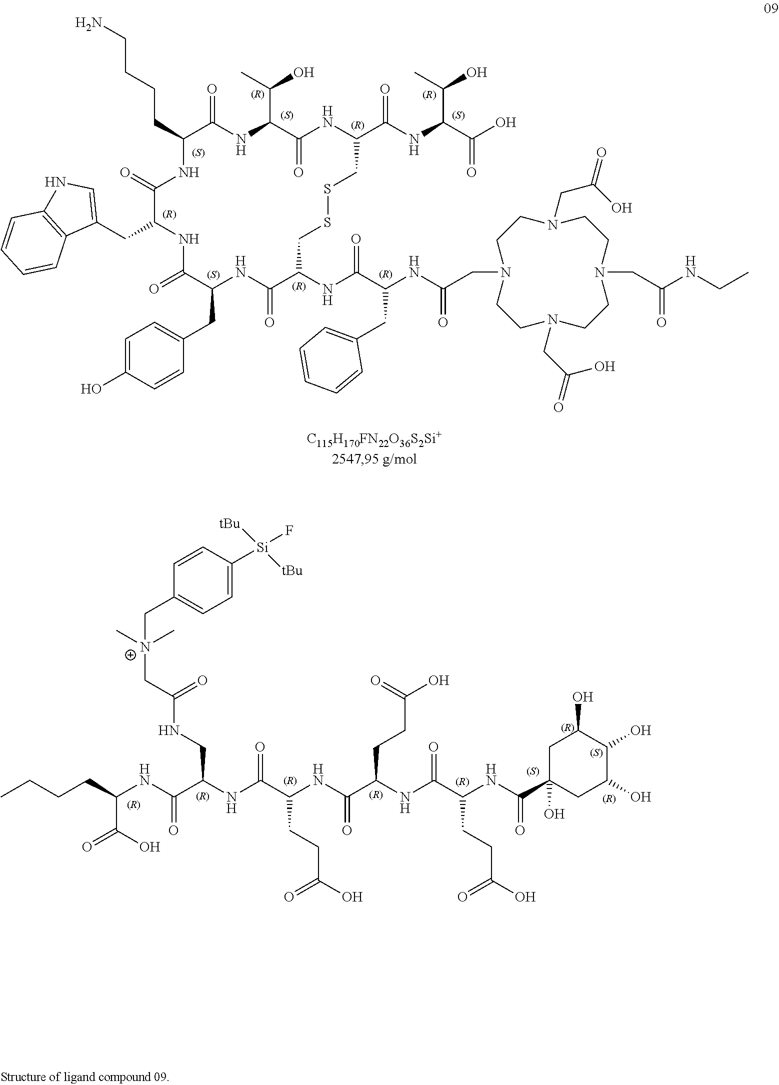

09 is synthesized starting from Fmoc-TATE (PG)-2-CT precursor described in Chapter 4. The precursor is Fmoc-deprotected (GSP2) and coupled to DOTA((Bu)₂ (GSP7). A solution of TBTU (1.5 eq.), HOAt (1.5 eq.), and DIPEA (4.5 eq.) in DMF (2.5 ml) is added to the resin and preactivated for 10 min. A solution of Fmoc-D-Lys-OfBu (1.5 eq.) in DMF (2.5 ml) is added to the preactivated resin and stirred for 2 h. The resin is washed with DMF (6×5 ml) and Fmoc-D-Dap(Dde)-OH is coupled (GSP6). The Dde- group is cleaved (GSP3) and dimethylglycine hydrochloride is coupled (GSP4). After Fmoc-deprotection (GSP2), Fmoc-D-Glu((Bu)-OH is coupled (GSP4), followed by Fmoc-D-Glu(fBu)-OH (GSP2, GSP4) and Fmoc-D-Glu((Bu)-OH (GSP2, GSP4). After Fmoc-deprotection (GSP2), quinic acid is coupled twice (2×GSP4) followed by SiFA-Br (GSP8). The compound is cleaved from the resin with cleavage of all acid-labile protecting groups (GSP11), purified via preparative RP-HPLC, and lyophilized (GSP13). The formation of the correct product is confirmed by QC using analytical RP-HPLC and ESI-MS.

09 (D-(−)-quinic acid-D-Glu-D-Glu-D-Glu-D-Dap(N-SiFAlin-N,N-Me2-Gly)-D-Lys(trans-DOTA-TATE)-OH):

RP-HPLC (analytical): (10-60% MeCN/H₂O with 0.1% TFA, v/v, 15 min): t_{R}=11.6 min; K′=4.5.

RP-HPLC (preparative): (38-42% MeCN/H₂O with 0.1% TFA, v/v, 30 min): t_{R}=24.4 min; K′=3.4.

MS (ESI positive): m/z calculated for C₁₁₅H₁₇₀FN₂₂₀₃₆S₂Si⁺: 2546.13, found: 849.1 [M+3H]³⁺, 1272.9 [M+2H]²⁺, 1696.8 [2M+3H]³⁺.

### 5. Complexation with ^{nat}Ga or ^{nat}Lu

Complexation with ^{nat}Ga or ^{nat}Lu occurs according to GSP12 or GSP14. The formation of the correct product is confirmed by QC applying analytical RP-HPLC and ESI-MS.

^{nat}Ga] 01:

### N-SiFAlin-N,N-Me2-Gly-D-Lys(trans-[^{nat}Ga] DOTA-TATE)-OH

RP-HPLC (analytical): (10-60% MeCN/H₂O with 0.1% TFA, v/V, 15 min): t_{R}=13.2 min; K′=5.3.

MS (ESI positive): m/z calculated for C₉₀H₁₃₁FGaN₁₇O₂₁S₂Si⁺: 1965.82, found: 656.2 [M+3H]³⁺, 983.8 [M+2H]²⁺, 1311.8 [2M+3H] ³⁺,

^{nat}Ga102:

### N-SiFAlin-N, N-Me2-Gly-D-Dap(trans-[^{nat}Ga] DOTA-TATE)-OH

RP-HPLC (analytical): (10-60% MeCN/H₂O with 0.1% TFA, v/V, 15 min): t_{R}=13.1 min; K′=5.2.

MS (ESI positive): m/z calculated for C₈₇H₁₂₅FGaN₁₇O₂₁S₂Si⁺: 1923.77, found: 642.2 [M+3H]³⁺, 962.7 [M+2H]²⁺, 1283.5 [2M+3H]3+

^{nat}Ga103:

H-D -Cit-D -Cit-D -Cit-D-Dap(N-SiFAlin-N,N-Me2-Gly)-D-Lys(trans-[na′Ga] DOTA-TATE)-OH

RP-HPLC (analytical): (10-60% MeCN/H₂O with 0.1% TFA, v/v, 15 min): t_{R}=11.5 min; K=4.5.

MS (ESI positive): m/z calculated for C₁₁₁H₁₇₀FGaN₂₈O₂₈S₂Si⁺: 2523.12, found: 841.7 [M+3H]³⁺, 1262.3 [M+2H]²⁺, 1682.8 [2M+3H] ³⁺. ^{nat}Ga104:

H-D-Glu-D -Cit-D -Cit-D-Dap(N-SiFAlin-N,N-Mez-Gly)-D-Lys(trans-[^{nat}Ga] DOTA-TATE)-OH

RP-HPLC (analytical): (10-60% MeCN/H₂O with 0.1% TFA, v/v, 15 min): t_{R}=11.5 min; K′=4.5.

MS (ESI positive): m/z calculated for C₁₁₀H₁₆FGaN26029S₂Si⁺: 2495.08, found: 832.3 [M+3H]³⁺, 1248.3 [M+2H]²⁺, 1664.2 [2M+3H]³⁺.

^{nat}Ga105:
H-D-Glu-D-Glu-D-Dap-D-Dap(N-SiFAlin-N,N-Me2-Gly)-D-Lys(trans-[^{nat}Ga] DOTA-TATE)-OH RP-HPLC (analytical): (10-60% MeCN/H₂O with 0.1% TFA, v/v, 15 min): t_{R}=11.3 min; K′=4.4.

MS (ESI positive): m/z calculated for C₁₀₆H₁₅₇FGaN₂₃O₂₉SzSi⁺: 2396.00, found:

600.3 [M+4H]⁴⁺, 799.7 [M+3H]³⁺, 1199.1 [M+2H]²⁺, 1598.5 [2M+3H]³⁺.

[^{nat}Lu] 05:

H-D-Glu-D-Glu-D-Dap-D-Dap(N-SiFAlin-N,N-Me2-Gly)-D-Lys(trans-[^{nat}Lu] DOTA-TATE)-OH

RP-HPLC (analytical): (10-90% MeCN/H₂O with 0.1% TFA, v/v, 15 min): t_{R}=8.10 min; K′=4.4.

MS (ESI positive): m/z calculated for C₁₀₆H₁₅₇FLuN₂₃O₂₉S₂Si⁺: 2502.01, found: 834.6 [M+3H]³⁺, 861.0 [M+DMSO+3H]³⁺125.8 [M+2H]2+

^{nat}Ga106:

H-D-Glu-D-Glu-D-Lys-D-Dap(N-SiFAlin-N, N-Me2-Gly)-D-Lys(trans-[^{nat}Ga] DOTA-TATE)-OH

RP-HPLC (analytical): (10-60% MeCN/H₂O with 0.1% TFA, v/v, 15 min): t_{R}=11.2 min; K′=4.3.

MS (ESI positive): m/z calculated for C₁₀₉H₁₆₃FGaN₂₃O₂₉S₂Si⁺: 2438.04, found: 610.6 [M+4H]⁴⁺, 813.9 [M+3H]³⁺, 1220.3 [M+2H]²⁺, 1626.7 [2M+3H] ³⁺.

[^{nat}Ga] 07:

H-D-Glu-D-Glu-D-Glu-D-Dap(N-SiFAlin-N,N-Me2-Gly)-D-Lys(trans-[^{nat}Ga] DOTA-TATE)-OH

RP-HPLC (analytical): (10-60% MeCN/H₂O with 0.1% TFA, v/v, 15 min): t_{R}=11.7 min; K′=4.6.

MS (ESI positive): m/z calculated for C₁₀₈H₁₅₈FGaN22031S₂Si⁺: 2438.99, found: 610.2 [M+4H]⁴⁺, 813.2 [M+3H]³⁺, 1219.2 [M+2H]²⁺, 1625.4 [2M+3H]³⁺, 1828.7 [3M+4H]^{4 +}. ^{nat}Ga] 08:

H-D-Glu-D-Glu-D-Glu-D-Dap(N-SiFAlin-N,N-Me2-Gly)-D-Glu-D-Lys(trans-[^{nat}Ga] DOTA-TATE)-OH

RP-HPLC (analytical): (10-60% MeCN/H₂O with 0.1% TFA, v/v, 15 min): t_{R}=11.5 min; K′=4.5.

MS (ESI positive): m/z calculated for C₁₁₃H₁₆₅FGaN₂₃O₃₄S₂Si⁺: 2568.03, found: 856.4 [M+3H]³⁺, 1283.7 [M+2H]²⁺, 1711.6 [2M+3H] ³⁺.

^{nat}Ga] 09:

D-(−)-quinic acid-D-Glu-D-Glu-D-Glu-D-Dap(N-SiFAlin-N,N-Me2-Gly)-D-Lys(trans-[^{nat}Ga] DOTA-TATE)-OH

RP-HPLC (analytical): (10-60% MeCN/H₂O with 0.1% TFA, v/v, 15 min): t_{R}=11.9 min; K′=4.7.

MS (ESI positive): m/z calculated for C₁₁₅H₁₆₈FGaN22036S₂Si⁺: 2613.04, found: 871.7 [M+3H]³⁺, 1306.9 [M+2H]²⁺, 1742.3 [2M+3H]³⁺, 1960.5 [3M+4H]⁴⁺.

MS (ESI positive): m/z calculated for C₁₁₇H₁₇₆FGaN26034S₂Si⁺: 2669.13, found: 890.3 [M+3H]³⁺, 1334.9 [M+2H]²⁺, 1779.5 [2M+3H]³⁺

### 6. Synthesis of Radioactive Ligands

### 6.1 Synthesis of [¹²⁵I] TOC

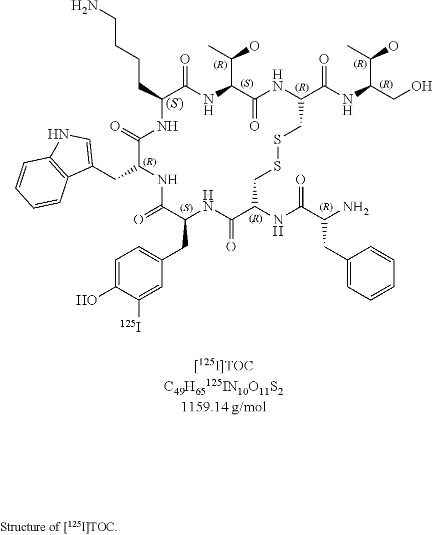

The reference ligand for in vitro studies [125] TOC was prepared according to a previously published procedure.[21] Briefly, 50-150 μg of the uniodinated precursor TOC were dissolved in 20 μL DMSO and 280 μL TRIS iodination buffer (25 mm TRIS-HCl, 0.4 mm NaCl, PH=7.5). After addition of 5.00 μL (15-20 MBq) [¹²⁵I] Nal (74 TBq/mmol, 3.1 GBq/mL, 40 mM NaOH, Hartmann Analytic, Braunschweig, Germany) the solution was transferred to a reaction vial, coated with 150 μg IodoGen®. The reaction was incubated for 15 min at RT and stopped by separation of the solution from the oxidant. The crude product of [125] I-TOC was purified by RP-HPLC [(20% to 50% B in 15 min): t_{R}=9.4 min] and the final, dissolved product was treated with 10 Vol-% of a 100 mm solution of Na-ascorbate in H₂O to prevent radiolysis.
6.2 18F-Fluorination by an Ion Exchange Reaction
For ¹⁸F-labeling a previously published procedure was applied.
   7. In vitro Experiments

### 7.1 Cell Culture

Before the cultivation of AR42J or CHO-SST₂-cells, all used biochemicals are heated to 37° C.

### Cultivation of AR42J Cells

The AR42J cells were cultivated in RPMI 1640 medium (with 2 mM L-Glu, 10% FCS; v/v) and incubated at 37° C. in a humidified 5% CO₂ atmosphere. To ensure a constant rate of cellular growth, the cells were split every 3-4 days.

The depleted medium was discarded, and the adherent cells washed with PBS (10 ml). The cells are then dislodged from the cell culture flask by treatment with 0.1% EDTA in PBS (5 ml, min, 37° C.ₒ) and resuspended with the addition of 5 ml of RPMI 1640 medium (with 2 mM L-Glu, 10% FCS; v/v). The suspension is centrifuged (1300 revolutions per minute (rpm), 3 min, RT), the supernatant discarded, and the cell pellet resuspended in fresh RPMI 1640 medium (with 2 mM L-Glu, 10% FCS; v/v) medium. 10-50% of the suspension is transferred to a new cell culture flask and the volume topped off to 25 ml with fresh RPMI 1640 medium (with 2 mM L-Glu, 10% FCS; v/V) medium.

For internalization evaluations, the cell-pellet is resuspended in 20 ml RPMI 1640 medium (with 2 mM L-Glu, 10% FCS; v/v). 10 ul of the suspension are mixed with 10 ul of trypan blue solution. 10 ul of the resulting mixture are given to a Neubauer-counting chamber (0.1 mm dept, 0.0025 mm2 area). The cells are counted under a light microscope and the cell concentration of the 20 ml suspension determined according to the following formula:

$$\frac{cells}{ml} = \frac{counted cells}{4} \cdot 20 \text{,} 000$$

The cells were then seeded into 24-well poly-L-lysine plates (2.0×10⁵cells) and incubated in 1 ml of RPMI 1640 medium (with 2 mM L-Glu, 10% FCS; v/v) for 24±2 h at 37° C. in a humidified 5% CO₂ atmosphere.

### Cultivation of CHO-SST₂ Cells

The SST₂-transfected CHO-SST₂ cells were cultivated in DMEM/F12, (with 10% FCS; v/V) and incubated at 37° C. in a humidified 5% CO₂ atmosphere. To ensure a constant rate of cellular growth, the cells were split every 2-3 days.

The depleted medium was discarded, and the adherent cells washed with PBS (10 ml). The cells are then dislodged from the cell culture flask by treatment with trypsin/EDTA (5 ml, 5 min, 37° C.ₒ) and resuspended with the addition of 5 ml of DMEM/F-12 (with 10% FCS; v/v) medium.

The suspension is centrifuged (1300 rpm, 3 min, RT), the supernatant discarded, and the cell-pellet resuspended in 20 ml of fresh DMEM/F-12 (with 10% FCS; v/v) medium. A part of the suspension is transferred to a new cell culture flask and the volume topped off to 25 ml with fresh DMEM/F-12 (with 10% FCS; v/v) medium.

For the determination of IC₅₀ values, the cell-pellet is resuspended in 20 ml DMEM/F-12 (with 10% FCS; v/v) medium. 10 ul of the suspension are mixed with 10 ul of trypan blue solution. 10 μl of the resulting mixture are given to a Neubauer-counting chamber (0.1 mm dePt, 0.0025 mm2 area). The cells are counted under a light microscope and the cell concentration of the 20 ml suspension determined according to the following formula:

$$\frac{cells}{ml} = \frac{counted cells}{4} \cdot 20 \text{,} 000$$

The cells were then seeded into 24-well plates (1.0×10⁵cells) and incubated in 1 ml of DMEM/F-12 (with 10% FCS; v/v) medium for 24±2 h at 37° C. in a humidified 5% CO₂ atmosphere.

### 7.2 IC₅₀ Evaluation

The SST₂-transfected CHO cells were cultivated in DMEM/F12 (with 10% FCS) and incubated at 37° C. in a humidified 5% CO₂ atmosphere. For the determination of the IC₅₀, cells were harvested 24±2 h before the experiment, seeded in 24-well plates (1.0×10⁵cells), and incubated in 1 ml/well of culture medium.

After removal of the culture medium, the cells are washed once with 400 ul of HBSA and 200 μl of fresh HBSA are added. Next, 25 μl of either HBSA (control) of the respective ligand in increasing concentrations (10-10-10-4 M in HBSA) were added with subsequent addition of 25 μl of [125] TOC (1.0 nm in HBSA) per well. Each concentration is investigated as a triplicate. After 60 min incubation at RT, the experiment was terminated by the removal of the assay medium and subsequent washing with 300 μl of cold PBS. The media of both steps were combined in one fraction and represent the amount of unbound radioligand. Afterward, the cells were lysed with 300 μl of 1 M NaOH (15 min, RT) and united with the 300 μl 1 M NaOH of the following wash step.

Quantification of bound and unbound radioligand was accomplished in a γ-counter. The mathematical analysis was carried out using the GraphPad PRISM software.

### 7.3 Internalization Studies

The AR42J cells were cultivated in RPMI 1640 medium (with 2 mM L-Glu, 10% FCS; v/v) and incubated at 37° C. in a humidified 5% CO₂ atmosphere. For the quantification of the internalization, cells were harvested 24±2 h before the experiment, seeded in 24-well poly-L-lysine plates (2.0×10⁵cells), and incubated in 1 ml/well of culture medium. After removal of the culture medium, the cells were washed with 300 μl of assay medium (RPMI 1640 medium with 2 mM L-Glu, 5% BSA; v/v) and preincubated at 37° C. in 200 μl of assay medium for at least 15 min. 25 μl of a mixture of the ¹⁸F-labeled ligand (20 nm) and ¹²⁵I -TOC (1 nM) in assay medium is added to the wells followed by either 25 μl of TOC in assay-medium (100 UM, competition experiment) or 25 μl of assay medium (internalization experiment). One 24-well plate per investigated time (15, 30, and 60 min) is incubated for the respective time (37° C., 5% CO₂). The plate is chilled on ice, the supernatant collected, and the well washed with 300 μl of ice-cold wash solution (RPMI 1640 medium) which is combined with the supernatant. 300 μl of acid wash solution (0.9% NaCl, 50 mM sodium acetate/acetic acid buffer, pH=4.6) is added and incubated on ice for 15 min. The supernatant is collected, and the cells are washed with 300 μl of ice-cold acid wash solution. 300 μl of aqueous NaOH solution (1 M) is added to the cells and incubated for at least 15 min at RT. The solution is collected and the well washed with 300 μl of the NaOH solution.

The ¹⁸F-activity is quantified in a γ-counter followed by the ¹²⁵I -activity of the same samples.

8. In vivo Experiments

### 8.1 Mouse Model and Tumor Model

All animal experiments were conducted in accordance with general animal welfare regulations in Germany and the institutional guidelines for the care and use of animals. To establish tumor xenografts, AR42J cells (5×10⁶ cells/100 μL) were suspended in Dulbecco modified Eagle medium/Nutrition Mixture F-12 with Glutamax-I (1:1) and inoculated subcutaneously onto the right shoulder of 8 weeks old, female CD1 nu/nu mice (Charles River, Sulzfeld, Germany). Mice were used for experiments when tumors had grown to a diameter of 5-9 mm (7-15 days after inoculation).

### 8.2 Biodistribution

Approximately 0.5-2.0 MBq (0.05-0.20 nmol) of the ¹⁸F-labeled SST₂-ligands were injected into the tail vein of AR42J tumor-bearing female CD1 nu/nu mice. The mice were sacrificed 1 h post injection (n=3-5). Selected organs were removed, weighed, and measured in a γ-counter.

### 9. Further Investigations

### 9.1 HSA Binding Studies

### RIAC Method

A gel filtration column Superdex 75 Increase 10/300 GL (GE Healthcare, Uppsala, Sweden) was beforehand calibrated following the producer's recommendations with a commercially available gel filtration calibration kit (GE Healthcare, Buckinghamshire, UK) comprising conalbumin (MW: 75 kDa), ovalbumin (44 kDa), carbonic anhydrase (29 kDa), ribonuclease A (13.7 kDa) and aprotinin (6.5 kDa) as reference proteins of known molecular weight. AMSEC experiments were conducted using a constant flow rate of 0.8 mL/min at rt. A solution of HSA in PBS at physiological concentration (700 μM) was used as the mobile phase. PSMA ligands were labelled as described with molar activities of 10-20 GBq/μmol. Probes of 1.0 MBq of the radioligand were injected directly from the labelling solution. HSA binding was expressed as an apparent molecular weight MW calculated from the retention time of the radioligand using the determined calibration curve.

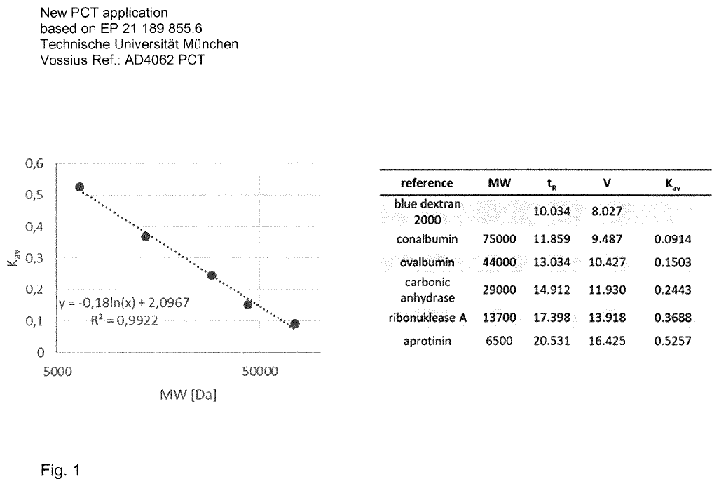

FIG. 1 shows the calibration plot of Superdex 75 Increase gel filtration column using a low molecular weight gel filtration calibration kit. MW: molecular weight. t_{R}: experimentally determined retention time. V: elution volume. Kₐᵥ: partition coefficient.

For evaluation, experimentally determined retention times t_{R} are first converted into elution volumes Vₑ by multiplying with the flow rate and thereafter converted into partition coefficients Kₐᵥ following the equation

$${K}_{av} = \frac{{V}_{e} - {V}_{0}}{{V}_{c} - {V}_{0}}$$

where V₀ is the column void volume (8.027 mL) and V_{c} is the geometric column volume (24 mL). Using the equation given by the trend line plot of the column calibration

$${K}_{aν} = - 0.18 ln ( M W ) + 2.096 \; 7$$

the apparent molecular weight MW is calculated as

$$MW = {e}^{\frac{2.0967 - {K}_{av}}{0.18}}$$

### 9.2 Octanol-water Distribution Coefficient

The radioactive ligand of interest (0.7-1.0 MBq) is given to a mixture of n-octanol and PBS (1 ml, 1/1; v/V) in a 1.5 ml reaction tube and shaken vigorously for 3 min. The resulting mixture is centrifuged (9000 rpm, 5 min, RT), and 100 μl of the octanol and PBS phases isolated separately.

Quantification occurs by the determination of the activity of each isolated probe in a γ-counter. The LogD_{pH=7.4} value is determined by the following equation:

$${LogD}_{p H = 74} = {log}_{10} \frac{cpm ( octanol )}{cpm ( PBS )}$$

The determination of the final LogD_{pH=7.4} value is executed in octets. The mean value and standard deviation are determined after the removal of outlying values.

### II. Results

| | | | | Internalisation |
|---|---|---|---|---|
| | | | HSA | in % relative |
| Compound | IC₅₀ [nM] | LogD_{pH=7.4} | [kDa] | to [¹²⁵I]TOC |
| [Ga]01 | 5.69 ± 0.23 | −1.19 ± 0.07 | | |
| [Ga]02 | 5.59 ± 1.38 | −1.03 ± 0.04 | | |
| 03 | 9.24 ± 1.39 | | | |
| [Ga]03 | 3.69 ± 0.54 | −2.27 ± 0.04 | 6656 | 536 |
| 04 | 10.58 ± 1.01 | | | |
| [Ga]04 | 3.31 ± 0.51 | −2.30 ± 0.03 | 6023 | 550 |
| 05 | 7.38 ± 1.01 | −1.96 ± 0.06 | | |
| [Ga]05 | 2.57 ± 0.20 | −2.12 ± 0.06 | 6678 | 619 |
| 06 | 5.93 ± 1.14 | −2.36 ± 0.03 | | |
| [Ga]06 | 3.17 ± 0.56 | −2.28 ± 0.04 | 6794 | 841 |
| 07 | 12.07 ± 0.74 | | | |
| [Ga]07 | 2.75 ± 0.20 | −2.30 ± 0.04 | 7068 | 357 |
| [Ga]08 | 4.06 ± 0.22 | −2.07 ± 0.04 | 5687 | 707 |
| [Ga]09 | 5.62 ± 0.25 | −2.28 ± 0.04 | 7038 | 335 |

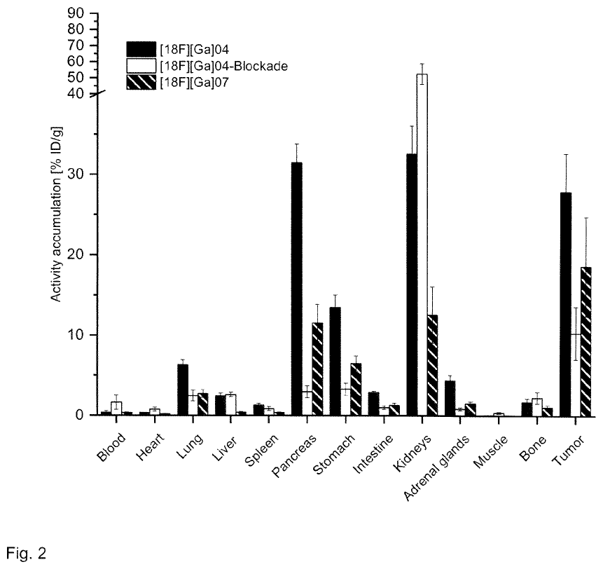

Results of the biodistribution study are shown in FIG. 2.

1. A compound selected from:
(a) a compound of formula (I) 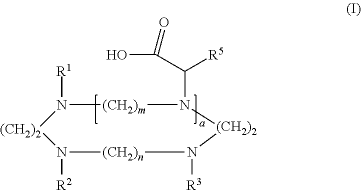
wherein
a is 0 or 1;
m is 2 or 3;
n is 2 or 3;
one group selected from R¹, R² and R³ is a group comprising an effector moiety R^{B};
another group selected from R¹, R² and R³ is a group comprising a silicon-based fluoride acceptor (SiFA) moiety R^{S}, which moiety comprises a silicon atom and a fluorine atom, wherein the fluorine atom is linked via a covalent bond directly to the silicon atom, and which can be labeled with ¹⁸F by isotopic exchange of ¹⁹F by ¹⁸F or which is labeled with ¹⁸F;
and the remaining group selected from R¹, R² and R³ is a group of the formula (R-1): 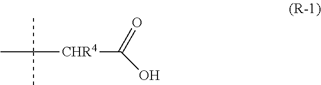
wherein
R⁴ is selected from —H, —OH and C₁-C₃ alkyl; and wherein the dashed line marks a bond which attaches the group to the remainder of the compound; and
R⁵ is selected from —H, —OH and C₁-C₃ alkyl;
(b) a salt thereof; and
(c) a chelate compound formed from a compound of formula (I) or its salt, and a radioactive or non-radioactive cation.

2. The compound of claim 1, wherein the SiFA moiety R^{S} comprises a group of the formula (S-2): 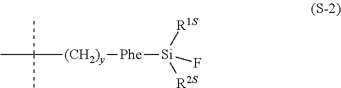
wherein
R^{1S} and R^{2S} are independently from each other a linear or branched C₃ to C₁₀ alkyl group;
Phe is a phenylene group;
y is an integer of 0 to 6; and
wherein the dashed line marks a bond which attaches the group to the remainder of the compound.

3. The compound of claim 2, wherein the SiFA moiety R^{S} is a group of the formula (S-3): 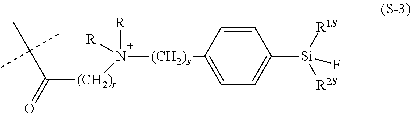
wherein
r is 1, 2 or 3, s is an integer of 1 to 6;
each R is independently C1 to C6 alkyl;
R^{1S} and R^{2S} are independently from each other a linear or branched C3 to C10 alkyl group; and
wherein the dashed line marks a bond which attaches the group to the remainder of the compound.

4. The compound of claim 1, wherein the effector moiety R^{B} is a peptidic binding motif which is able to bind to a receptor.

5. The compound of claim 4, wherein R^{B} is a peptidic binding motif which is able to bind to a somatostatin receptor.

6. The compound of claim 5, wherein R^{B} is a moiety which can be derived from a receptor agonist or receptor antagonist selected from Tyr³-Octreotate (TATE, H-D-Phe-cyclo(L-Cys-L-Tyr-D-Trp-L-Lys-L-Thr-L-Cys)-L-Thr-OH), Thr⁸-Octreotide (ATE), Phe¹-Tyr³-Octreotide (TOC, H-D-Phe-cyclo(L-Cys-L-Tyr-D-Trp-L-Lys-L-Thr-L-Cys)-L-Thr-ol), NaI³-Octreotide (NOC, H-D-Phe-cyclo(L-Cys-L-1-Nal-D-Trp-L-Lys-L-Thr-L-Cys)-L-Thr-ol), 1-NaI³,Thr⁸-Octreotide (NOCATE), BzThi³-Octreotide (BOC), BzThi³,Thr⁸-Octreotide (BOCATE), JR11 (H-L-Cpa-cyclo(D-Cys-L-Aph(Hor)-D-Aph (Cbm)-L-Lys-L-Thr-L-Cys)-D-Tyr-NH₂), BASS (H-L-Phe (4-NO₂)-cyclo(D-Cys-L-Tyr-D-Trp-L-Lys-L-Thr-L-Cys)-D-Tyr-NH₂) and KE121 (cyclo(D-Dab-L-Arg-L-Phe-L-Phe-D-Trp-L-Lys-L-Thr-L-Phe).

7. The compound of claim 1, wherein the group comprising an effector moiety R^{B} is a group of the formula (R-2a) or (R-2b), preferably of the formula (R-2a): 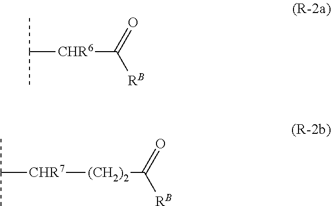
wherein
R^{B} is as defined in claim 1;
R⁶ is selected from —H, —OH and C1-C3 alkyl, and is preferably —H;
R⁷ is —COOH; and
wherein the dashed line marks a bond which attaches the group to the remainder of the compound.

8. The compound of claim 7, wherein the group comprising the SiFA moiety R^{S} is a group of the formula (R-3a), (R-3b), (R-3c) or (R-3d), preferably of the formula (R-3a) or (R-3b). 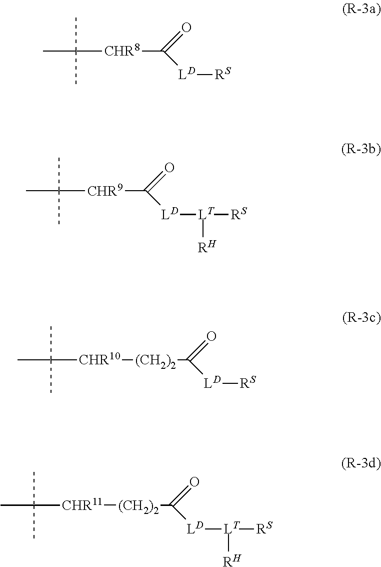
wherein
R^{S} is as defined in claim 7;
R⁸ and R⁹ are selected from —H, —OH and C1-C3 alkyl, and are preferably —H;
R¹⁰ and R¹¹ are —COOH;
L^{D} is a divalent linking group;
L^{T} is a trivalent linking group;
R^{H} is a hydrophilic modifying group; and
wherein the dashed line marks a bond which attaches the group to the remainder of the compound.

9. The compound of claim 8, wherein the compound of formula (I) is a compound of formula (IC): 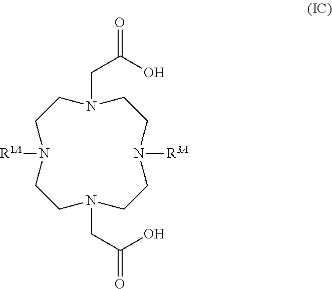
wherein
i) R^{1A} is a group of formula (R-2a) as defined in claim [7] 8 and R^{3A} is selected from the groups of formula (R-3a), (R-3b), (R-3c) and (R-3d) as defined in claim 8; or
ii) R^{1A} is selected from the groups of formula (R-2a) and (R-2b) as defined in claim 8 and R^{3A} is selected from the groups of formula (R-3a) and (R-3b) as defined in claim 8.

10. The compound of claim 1, wherein the compound of formula (I) is a compound of formula (ID) or (IE): 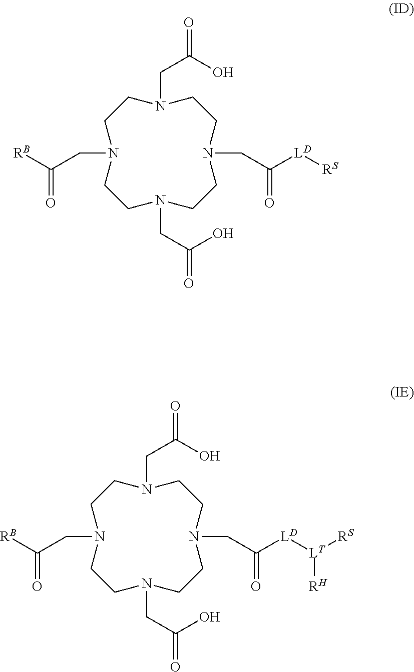
wherein
R^{B} and R^{S} are as defined in claim 1;
L^{D} is a divalent linking group;
L^{T} is a trivalent linking group; and
R^{H} is a hydrophilic modifying group.

11. The compound of claim 10, wherein R^{B} is a moiety which can be derived from a receptor agonist or receptor antagonist selected from Tyr³-Octreotate (TATE, H-D-Phe-cyclo(L-Cys-L-Tyr-D-Trp-L-Lys-L-Thr-L-Cys)-L-Thr-OH), Thr⁸-Octreotide (ATE), Phe¹-Tyr³-Octreotide (TOC, H-D-Phe-cyclo(L-Cys-L-Tyr-D-Trp-L-Lys-L-Thr-L-Cys)-L-Thr-ol), NaI³-Octreotide (NOC, H-D-Phe-cyclo(L-Cys-L-1-Nal-D-Trp-L-Lys-L-Thr-L-Cys)-L-Thr-ol), 1-NaI³,Thr⁸-Octreotide (NOCATE), BzThi³-Octreotide (BOC), BzThi³,Thr⁸-Octreotide (BOCATE), JR11 (H-L-Cpa-cyclo(D-Cys-L-Aph(Hor)-D-Aph (Cbm)-L-Lys-L-Thr-L-Cys)-D-Tyr-NH₂), BASS (H-L-Phe (4-NO₂)-cyclo(D-Cys-L-Tyr-D-Trp-L-Lys-L-Thr-L-Cys)-D-Tyr-NH₂) and KE121 (cyclo(D-Dab-L-Arg-L-Phe-L-Phe-D-Trp-L-Lys-L-Thr-L-Phe); and
R^{S} is a group of the formula (S-3): 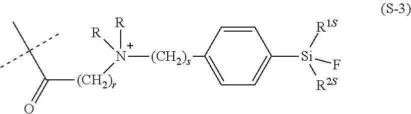
wherein
r is 1, 2 or 3, s is an integer of 1 to 6;
R is independently C1 to C6 alkyl;
R^{1S} and R^{2S} are both tert-butyl; and
wherein the dashed line marks a bond which attaches the group to the remainder of the compound.

12. The compound of claim 8, wherein the divalent linking group LP is a group of formula (L-2): 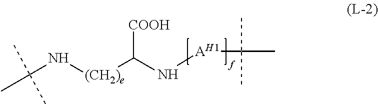
wherein
e is an integer of 1 to 6, preferably 1 to 4;
f is an integer of 0 to 5, preferably 0 or 1;
each A^{H1} is independently for each occurrence if f is more than 1, an amino acid unit derived from a hydrophilic amino acid which comprises a further hydrophilic functional group in addition to its —NH₂ and its —COOH functional group;
the dashed lines mark bonds which attach the group to adjacent groups; and
the bond additionally marked by the asterisk is attached to R^{S} or RT, respectively.

13. The compound of claim 8, wherein the hydrophilic modifying group -R^{H} is a group of formula (H-1): 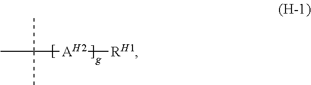
wherein
g is an integer of 0 to 5, preferably 1 to 3;
each A^{H2} is independently for each occurrence if g is more than 1, an amino acid unit derived from a hydrophilic amino acid which comprises a further hydrophilic functional group in addition to its —NH₂ and its —COOH functional group;
R^{H1} is selected from a terminal hydrogen atom attached to an amino acid unit A^{H2}, an acetyl group or a hydrophilic unit selected from a carbohydrate group, a polyvalent alcohol unit and a polyvalent carboxylic acid unit; and
the dashed line marks a bond which attaches the group to the remainder of the compound.

14. The compound of claim 1, wherein the radioactive or non-radioactive cation of the chelate compound is selected from the cations of ⁴³Sc, ⁴⁴Sc, ⁴⁷Sc, ⁵¹Cr, ⁵²mMn, ⁵⁵Co, ⁵⁷Co, ⁵⁸Co, ⁵²Fe, ⁵⁶Ni, ⁵⁷Ni, ⁶²Cu, ⁶⁴Cu, ⁶⁷Cu, ⁶⁶Ga, ⁶⁸Ga, ⁶⁷Ga, ⁸⁹Zr, ⁹⁰Y, ⁸⁶Y, ⁹⁴mTc, ^{99m}mTc, ⁹⁷Ru, ¹⁰⁵Rh, ¹⁰⁹Pd, ¹¹¹ Ag, ^{110m}In, ¹¹¹ In, ^{113m}In, ^{114m}In, ^{117m}Sn, ¹²¹Sn, ¹²⁷Te, ¹⁴²Pr, ¹⁴³Pr, ¹⁴⁷Nd, ¹⁴⁹Gd, ¹⁴⁹Pm, ¹⁵¹Pm, ¹⁴⁹Tb, ¹⁵²Tb, ¹⁵⁵Tb, ¹⁵³Sm, ¹⁵⁶Eu, ¹⁵⁷Gd, ¹⁵⁵Tb, ¹⁶¹ Tb, ¹⁶⁴Tb, ¹⁶¹ Ho, ¹⁶⁶Ho, ¹⁵⁷Dy, ¹⁶⁵Dy, ¹⁶⁶Dy, ¹⁶⁰Er, ¹⁶⁵Er, ¹⁶⁹Er, ¹⁷¹Er, ¹⁶⁶Yb, ¹⁶⁹Yb, ¹⁷⁵Yb, ¹⁶⁷Tm, ¹⁷²Tm, ¹⁷⁷Lu, ¹⁸⁶Re, ^{186g}Re, ¹⁸⁸Re, ¹⁸⁸ W, ¹⁹¹ Pt, ^{195m}Pt, ¹⁹⁴Tr, ¹⁹⁷Hg, ¹⁹⁸ Au, ¹⁹⁹ Au, ²¹²Pb, ²⁰³Pb, ²¹¹ At, ²¹²Bi, ²¹³Bi, ²²³Ra, ²²⁴Ra, ²²⁵ Ac, ²²⁶Th and ²²⁷Th, and from cations of non-radioactive isotopes thereof, or is a cationic molecule comprising ¹⁸F or ¹⁹F, such as ¹⁸F-[AIF] 2+, and is preferably selected from a cation of 6°Ga, ⁹⁰Y, or ¹⁷⁷Lu and from cations of non-radioactive isotopes of Ga, Y or Lu.

15. A pharmaceutical or diagnostic composition comprising or consisting of one or more compounds of claim 1.


SEQUENCE LISTING (10 sequences)

SEQ ID NO: 1 - protein, 8 aa, synthetic construct
  source 1..8: note = receptor agonist or receptor antagonist (ATE)
  SITE 1: note = D-amino acid
  DISULFID 2..7: note = disulfide bond
  SITE 4: note = D-amino acid
FCFWKTCT

SEQ ID NO: 2 - protein, 8 aa, synthetic construct
  source 1..8: note = receptor agonist or receptor antagonist (TATE)
  SITE 1: note = D-amino acid
  DISULFID 2..7: note = disulfide bond
  SITE 4: note = D-amino acid
FCYWKTCT

SEQ ID NO: 3 - protein, 8 aa, synthetic construct
  source 1..8: note = receptor agonist or receptor antagonist (TOC)
  SITE 1: note = D-amino acid
  DISULFID 2..7: note = disulfide bond
  SITE 4: note = D-amino acid
FCYWKTCT

SEQ ID NO: 4 - protein, 8 aa, synthetic construct
  source 1..8: note = receptor agonist or receptor antagonist (KE121)
  SITE 1: note = Dbu
  SITE 1: note = D-amino acid
  DISULFID 1..8: note = disulfide bond
  SITE 5: note = D-amino acid
XRFFWKTF

SEQ ID NO: 5 - protein, 8 aa, synthetic construct
  source 1..8: note = receptor agonist or recepor antagonist (NOC)
  SITE 1: note = D-amino acid
  DISULFID 2..7: note = disulfide bond
  SITE 3: note = 1-Naphthylalanine
  SITE 4: note = D-amino acid
FCXWKTCT

SEQ ID NO: 6 - protein, 8 aa, synthetic construct
  source 1..8: note = receptor agonist or receptor antagonist (NOC-ATE)
  SITE 1: note = D-amino acid
  DISULFID 2..7: note = disulfide bond
  SITE 3: note = 1-Naphthylalanine
  SITE 4: note = D-amino acid
FCXWKTCT

SEQ ID NO: 7 - protein, 8 aa, synthetic construct
  source 1..8: note = receptor agonist or receptor antagonist (BOC)
  SITE 1: note = D-amino acid
  DISULFID 2..7: note = disulfide bond
  SITE 3: note = 3-benzothienylalanine
  SITE 4: note = D-amino acid
FCXWKTCT

SEQ ID NO: 8 - protein, 8 aa, synthetic construct
  source 1..8: note = receptor agonist or receptor antagonist (BOC-ATE)
  SITE 1: note = D-amino acid
  DISULFID 2..7: note = disulfide bond
  SITE 3: note = 3-benzothienylalanine
  SITE 4: note = D-amino acid
FCXWKTCT

SEQ ID NO: 9 - protein, 8 aa, synthetic construct
  source 1..8: note = receptor agonist or receptor antagonist (JR11)
  SITE 2: note = D-amino acid
  DISULFID 2..7: note = disulfide bond
  SITE 3: note = 4-amino-L-hydroorotylphenylalanine
  SITE 4: note = 4-aminocarbamoylphenylalanine
  SITE 8: note = D-amino acid
  SITE 1: note = chloro-phenylalanine
XCXXKTCY

SEQ ID NO: 10 - protein, 8 aa, synthetic construct
  source 1..8: note = receptor agonist or receptor antagonist (BASS)
  SITE 1: note = 4-nitro-phenylalanine
  SITE 2: note = D-amino acid
  DISULFID 2..7: note = disulfide bond
  SITE 4: note = D-amino acid
  SITE 8: note = D-amino acid
XCYWKTCY